(19) United States
(12) Patent Application Publication
(10) Pub. No.: US 2024/0285628 A1
(43) Pub. Date: Aug. 29, 2024

(54) NON-PEPTIDE TARGETED THERAPEUTICS AND USES THEREOF

(71) Applicant: Crinetics Pharmaceuticals, Inc., San Diego, CA (US)

(72) Inventors: R. Scott STRUTHERS, San Diego, CA (US); Jian ZHAO, San Diego, CA (US); Yunfei ZHU, San Diego, CA (US)

(21) Appl. No.: 18/566,887

(22) PCT Filed: Jun. 8, 2022
(86) PCT No.: PCT/US2022/032707

§ 371 Date: Dec. 4, 2023

Related U.S. Application Data
(60) Provisional application No. 63/208,923, filed on Jun. 9, 2021.

(51) Int. Cl. A61K 31/513; A61K 31/519; A61K 31/53; A61P 35/00; C07D 251/72; C07D 405/14; C07D 417/14; C07D 487/04; C07D 491/22

(52) U.S. Cl. CPC A61K 31/513; A61K 31/519; A61K 31/53; A61P 35/00; C07D 251/72; C07D 405/14; C07D 417/14; C07D 487/04; C07D 491/22

(57) ABSTRACT

Described herein are non-peptide drug conjugates (NPDCs) that target tumor cells expressing cell surface peptide and protein G protein-coupled receptors and their use in the treatment and/or diagnosis of cancer.

### CROSS-REFERENCE

This application claims benefit of U.S. Provisional Patent Application No. 63/208,923 filed on Jun. 9, 2021, which is incorporated herein by reference in its entirety.

### FIELD OF THE INVENTION

Described herein are non-peptide drug conjugates (NPDCs) and methods of using such drug conjugates as cancer therapeutics, diagnostics, or both.

### BACKGROUND OF THE INVENTION

Neoplasms are abnormal growth of cells and cause enormous medical burdens, including morbidity and mortality, in humans. Neoplasms include benign or noncancerous neoplasms which do not display malignant features and are generally unlikely to become dangerous (e.g., adenomas); malignant neoplasms display features such as genetic mutations, loss of normal function, rapid division, and ability metastasize (invade) to other tissues; and neoplasms of uncertain or unknown behavior. Malignant neoplasms (i.e., cancerous solid tumors) are the leading cause of death in industrialized countries. Noncancerous neoplasms including benign adenomas can also cause significant morbidity and mortality. Although standard treatments can achieve significant effects in tumor growth inhibition and even tumor elimination, the applied drugs exhibit only minor selectivity for the malignant tissue over healthy tissue and their severe side effects limit their efficacy and use. Specific targeting of neoplastic cells without affecting healthy tissue is a major desire for effective solid tumor therapy. As one of 3 main classes of cell surface receptors, G protein-coupled receptors (GPCRs) are frequently overexpressed in tumor cells and are considered promising targets for selective tumor therapy. Despite the advances achieved with peptide-drug conjugate (PDC) therapeutics that target cell surface receptors, there is a significant need for therapeutics and diagnostics that overcome limitations imposed by peptide- and protein-based targeted therapeutics, such as inability to penetrate large solid tumors, instability to proteases and peptidases; unfavorable absorption, distribution, metabolism, and excretion (ADME) properties; and manufacturing challenges. Non-peptide ligands conjugated to suitable drug cargos or payloads represent a novel class of selective cancer therapeutics or diagnostics.

### SUMMARY OF THE INVENTION

Described herein are non-peptide drug conjugates and their use in the treatment of tumors. The present disclosure provides an alternative and improved method for the treatment of tumors. In some embodiments, the non-peptide drug conjugates disclosed herein provide an improved method for targeting tumor cells over traditional therapies that have narrow therapeutic indexes.

In one aspect, described herein is a compound of Formula (I), or a pharmaceutically acceptable salt thereof:

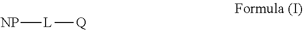

wherein:
(a) NP is a nonpeptide ligand that binds to a G protein-coupled receptor (GPCR) expressed in tumor cells;
(b) a payload moiety (Q) comprising a chemotherapeutic; and
(c) a linker (L) that covalently connects the non-peptide ligand NP and the payload moiety Q;
wherein the linker L is attached to NP at a position that permits binding of NP to the peptide or protein GPCR; and wherein upon administration to a mammal, the compound of Formula (I), or a pharmaceutically acceptable salt thereof, targets tumor cells expressing the GPCR.

In some embodiments, the GPCR is a receptor for an endogenous peptide or protein ligand. In some embodiments, the GPCR is a receptor for an endogenous peptide or protein hormone or a chemokine.

In some embodiments, NP is a small molecule that binds to a GPCR that recognizes an endogenous peptide or protein hormone that is: adrenocorticotropic hormone (ACTH), amylin, angiotensin, atrial natriuretic peptide (ANP), calcitonin, cholecystokinin (CCK), gastrin, ghrelin, glucagon, growth hormone (GH), follicle-stimulating hormone (FSH), insulin, leptin, melanocyte-stimulating hormone (MSH), oxytocin, parathyroid hormone (PTH), prolactin, renin, somatostatin, thyroid-stimulating hormone (TSH), thyrotropin-releasing hormone (TRH), vasopressin, or vasoactive intestinal peptide (VIP). In some embodiments, NP binds to a GPCR that recognizes an endogenous peptide or protein hormone, provided that the GPCR does not bind neurotensin.

In some embodiments, the GPCR is a chemoattractant GPCR. In some embodiments, the chemoattractant GPCR that is: a classical GPCR that is formyl peptide receptor (FPR1, FPR2, or FPR3), platelet activating factor receptor (PAFR), activated complement component 5a receptor (C5aR); or a chemokine GPCR that is binds to a CC chemokine (0-chemokine), CXC chemokine (α-chemokine), C chemokine (γ chemokine), or CX3C chemokine (d-chemokine).

In some embodiments, the GPCR is: an angiotensin receptor; apelin receptor; bombesin receptor; bradykinin receptor; calcitonin receptor; chemokine receptor; cholecytokinin receptor; corticotropic-releasing factor receptor; galanin receptor; ghrelin receptor; glucagon receptor; glycoprotein hormone receptor; gonadotropin-releasing hormone receptor; kisspeptin receptor; melanocortin receptor; motilin receptor; neuromedin U receptor; neuropeptide FF/AF receptor; neuropeptide S receptor; neuropeptide W/B receptor; neuropeptide Y receptor; opioid receptor; orexin receptor; parathyroid hormone receptor; prokineticin receptor; prolactin-releasing peptide receptor; QRFP receptor; relaxin family peptide receptor; somatostatin receptor; tachykinin receptor; thyrotropin-releasing hormone receptor; urotensin receptor; vasopressin and oxytocin receptor; VIP; or PACAP receptor.

In another aspect, described herein is a method for the treatment of tumors in a mammal comprising administering to the mammal a compound of Formula (I), or a pharmaceutically acceptable salt thereof. In some embodiments, the tumors comprise tumor cells expressing a GPCR. In some embodiments, the tissue(s) comprising the tumor cells also comprise non-tumor cells that do not express the GPCR or express the GPCR are lower expression levels than the tumor cells. In some embodiments, the the tumor cells overexpress the GPCR. In some embodiments, the GPCR expressed in the tumor cells of the tumor are targeted by the compound of Formula (I), or a pharmaceutically acceptable salt thereof. In some embodiments, the tumor cells are cells of a solid tumor, adenoma, sarcoma, carcinoma, or lymphoma. In some embodiments, the tumor cells are cells of a neoplasm.

In some embodiments, neoplasms, benign or malignant, are based on the type of cell origin and comprise solid tumors, adenomas, sarcomas, carcinomas, or lymphomas. In some embodiments, mammals with malignant neoplasms have anal cancer, bladder cancer, bowel cancer, brain cancer, breast cancer, colon cancer, colorectal cancer, endometrial cancer, esophageal cancer, gallbladder cancer, gastric cancer, heart cancer, kidney cancer, lung cancer, liver cancer, melanoma, uterine cancer, lymphoma, ovarian cancer, pancreatic cancer, or prostate cancer.

In some embodiments, the solid tumor is an endocrine tumor (i.e., endocrine in origin). In some embodiments, the endocrine tumor is an adrenal tumor, neuroendocrine tumor, parathyroid tumor, pituitary tumor, or thyroid cancer. In some embodiments, the tumor comprises neuroendocrine tumors. In some embodiments, the tumor comprises somatostatin receptor-positive gastroenteropancreatic neuroendocrine tumors (GEP-NETs).

In some embodiments, mammals with benign neoplasms have adenomas of the colon, kidney, adrenal gland, thyroid, pituitary, parathyroid, liver, breast, appendix, bronchial tube, prostate, sebaceous gland, or salivary gland.

In some embodiments, NP is a non-peptide ligand that binds to somatostatin receptors expressed in tumor cells, and wherein NP is a non-peptide ligand comprising a 4-(4-aminopiperidin-1-yl)-5-(phenyl)pyridine structural motif or a 4-[(4αS,8αS)-octahydro-1H-pyrido[3,4-b][1,4]oxazin-6-yl]-5-(phenyl)pyridine structural motif, wherein -L-Q is attached to NP at the 2-position of the pyridine.

In some embodiments, NP has a structure of Formula (II), or a pharmaceutically acceptable salt or pharmaceutically acceptable solvate thereof:

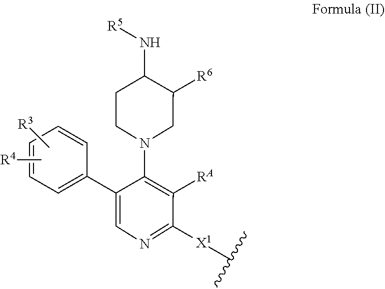

wherein:
R^{A} is

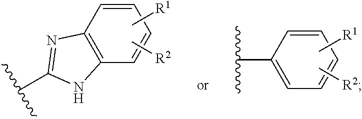

each R¹, R², R³ and R⁴ is independently hydrogen, halogen, substituted or unsubstituted C₁-C₄alkyl, substituted or unsubstituted C₁-C₄fluoroalkyl, substituted or unsubstituted C₁-C₄heteroalkyl, —CN, —N(R⁷)₂, or —OR⁷;
R⁵ is hydrogen, or substituted or unsubstituted C₁-C₆alkyl;
R⁶ is hydrogen, —OR⁷, —N(R⁷)₂, —CN, halogen, C₁-C₆ alkyl, or C₁-C₆fluoroalkyl;
or R⁵ and R⁶ are taken together with the intervening atoms to which they are attached to form a morpholine;
X¹ is absent, —O—, —S—, —N(R⁷)—, —C(═O)—, —C(═O)N(R⁷)—, —C(═O)O—, —N(R⁷)C(═O)—, or a heterocycle; and
each R⁷ is independently hydrogen or substituted or unsubstituted C₁-C₆alkyl.

In some embodiments, NP is a non-peptide ligand that binds to gonadotropin-releasing hormone receptor (GnRHR) expressed in tumor cells; and wherein NP is a non-peptide ligand comprising a N-{4,6-dimethoxy-pyrimidin-5-yl}-5-[3,3,6-trimethyl-2,3-dihydro-1H-inden-5-yl)oxy]-2-furamide structural motif, a N-(4,6-dimethoxypyrimidin-5-yl)-5-(3,3,6-trimethyl-2,3-dihydro-1H-inden-5-yl)oxy)-2-furamide structural motif, or a N-(4,6-dimethoxypyrimidin-5-yl)-5-((3,3,6-trimethyl-2,3-dihydro-1H-inden-5-yl)oxy)furan-2-carboxamide structural motif.

In some embodiments, NP has a structure of Formula (X), or a pharmaceutically acceptable salt or pharmaceutically acceptable solvate thereof:

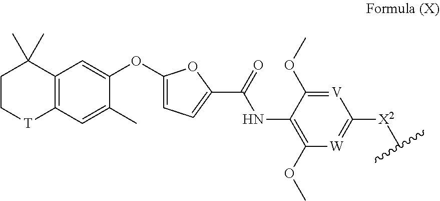

wherein:
V is CH or N; and W is CH or N;
T is absent, —CH₂—, —CH(CH₃)—, or —C(CH₃)₂—;
X² is absent, —O—, or —N(R⁷)—; and
R⁷ is hydrogen or substituted or unsubstituted C₁-C₆alkyl.

In yet another aspect, described herein is a method of targeting delivery of a chemotherapeutic to tumor cells in a mammal comprising administering a compound of Formula (I), or a pharmaceutically acceptable salt thereof, to a mammal with tumors:

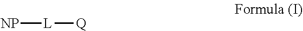

wherein:
(a) NP is a non-peptide ligand that binds to a G protein-coupled receptor (GPCR) expressed in tumor cells;
(b) a payload moiety (Q) comprising a chemotherapeutic; and
(c) a linker (L) that covalently connects the non-peptide ligand NP and the payload moiety Q.

In some embodiments, the tumor cells are present in tissues and/or organs that comprise non-tumor cells that do not express the targeted GPCR or express the targeted GPCR at levels that are less than the level of expression in the tumor cells. In some embodiments, the tumor cells overexpress a GPCR. In some embodiments, the tumor cells overexpress the GPCR targeted by the compound of Formula (I).

In some embodiments, L is a non-cleavable linker or a cleavable linker.

In some embodiments, NP is a non-peptide ligand that binds to a GPCR expressed in tumor cells, wherein the GPCR is a receptor for an endogenous peptide or protein hormone, and the tumor cells are tumor cells of a neoplasm, solid tumor, adenoma, sarcoma, carcinoma, or lymphoma; Q comprises a chemotherapeutic that is a cytotoxic drug, kinase inhibitor or both; L is optionally a non-cleavable linker or a cleavable linker. In some embodiments, the optional cleavable linker is an acid-sensitive linker, protease sensitivite linker, or glutathione-sensitivite linker.

In some embodiments, Q comprises a chemotherapeutic that is a ligand for an extracellular protein in the extracellular environment of the GPCR-expressing tumor cells. In some embodiments, Q comprises a cytotoxic drug that is an antimitotic, DNA-damaging agent, transcriptional inhibitor or combination thereof. In some embodiments, Q comprises a cytotoxic drug that is an antimitotic, and wherein the antimitotic is a maytansinoid, taxane, auristatin, alkaloid, tubulysin, or epothilone; the DNA-damaging agent, transcriptional inhibitor, or combination thereof is a DNA polymerase inhibitor, DNA replication inhibitor, topoisomerase inhibitor, cytotoxic antibiotic. In some embodiments, Q comprises a cytotoxic drug that is a DNA-damaging agent, transcriptional inhibitor, or combination thereof, wherein the DNA-damaging agent, transcriptional inhibitor, or combination thereof is a DNA polymerase inhibitor, DNA replication inhibitor, topoisomerase inhibitor, or cytotoxic antibiotic. In some embodiments, Q comprises a kinase inhibitor that is an inhibitor of a cytoplasmic tyrosine kinase (CTK), a serine/threonine kinase (S/T Kinase), a lipid kinase (LK), or a receptor tyrosine kinase (RTK).

In another aspect, described herein is a method for the treatment of cancer comprising administering to a mammal with cancer an effective amount of a compound of the compound of Formula (I), or a pharmaceutically acceptable salt thereof, or an effective amount of pharmaceutical composition comprising the compound of Formula (I), or a pharmaceutically acceptable salt thereof.

In another aspect, described herein is a method for treating tumors with a chemotherapeutic comprising administering to a mammal with tumors an effective amount of the compound of Formula (I), or a pharmaceutically acceptable salt thereof, or an effective amount of pharmaceutical composition comprising the compound of Formula (I), or a pharmaceutically acceptable salt thereof.

In some embodiments, the mammal has anal cancer, bladder cancer, bowel cancer, brain cancer, breast cancer, colon cancer, colorectal cancer, endometrial cancer, esophageal cancer, gallbladder cancer, gastric cancer, heart cancer, kidney cancer, lung cancer, liver cancer, melanoma, uterine cancer, lymphoma, ovarian cancer, pancreatic cancer, or prostate cancer.

In some embodiments, the mammal has an endocrine cancer. In some embodiments, the endocrine cancer is adrenal tumors, neuroendocrine tumors, parathyroid tumors, pituitary tumors, or thyroid tumors.

In some embodiments, the mammal has neuroendocrine tumors.

In some embodiments, the mammal has somatostatin receptor-positive gastroenteropancreatic neuroendocrine tumors (GEP-NETs).

In some embodiments, the tumors comprise an adenoma. In some embodiments, the adenoma is an adenoma of the colon, kidney, adrenal gland, thyroid, pituitary, parathyroid, liver, breast, appendix, bronchial tube, prostate, sebaceous gland, or salivary gland.

Also described herein is a pharmaceutical composition comprising a compound described herein, or a pharmaceutically acceptable salt, or solvate thereof, and at least one pharmaceutically acceptable excipient. In some embodiments, the pharmaceutical composition is formulated for administration to a mammal by intravenous administration, subcutaneous administration, or oral administration.

In any of the embodiments disclosed herein, the mammal is a human.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Cancer, a disease in which some cells undergo a genetic change in the control of their growth and replication that results in uncontroled growth and spreading, is one of the leading causes of death worldwide. General types of cancers include solid tumors (cancers that typically originate in organs), carcinomas (cancers that originate in skin or tissues that line organs), sarcomas (cancers of connective tissues such as bones), leukemias (cancers of bone marrow), and lymphomas and myelomas (cancers of the immune system). Neoplasms are abnormal growth of cells that result in solid tumors which may be benign (i.e. do not display malignant features and are generally unlikely to become dangerous such as adenomas), malignant (i.e. display features such as genetic mutations, loss of normal function, rapid division, and ability metastasize (invade) to other tissues), and of uncertain or unknown behavior. State-of-the-art treatment of neoplasms is accomplished by a combination of surgical procedures, chemotherapy, and radiation therapy. Surgical procedures can be curative under some conditions, but often requires multiple interventions as well as combination with radiation and chemotherapy. Chemotherapy proves to be a potent weapon in the fight against cancer in many cases, further optimization is required. Chemotherapy is typically performed by systemic administration of potent cytotoxic drugs, but these compounds lack tumor selectivity and therefore also kill healthy cells in the body. The resulting non-specific toxicity is the cause of severe side effects of chemotherapy which does not target the cancerous cells specifically over other cells. Radiotherapy is the use of high-energy radiation to kill cells. The source of radiation may be external-beam radiation (applied using an external source), internal radiation (placement of a radioactive material near the target cells), or radiotherapy from the systemic administration of a radioactive material. Similar to chemotherapy, many radiation therapy options also lack tumor cell identification properties needed to achieve the ultimate goal of targeted tumor therapy with drug molecules or radionuclides.

Described herein is the design of NPDCs that exploit characteristics that selectively identify neoplasms, such as significantly overexpressed cell surface receptors, different from healthy cells to achieve a therapeutic effect only in the selected cells. Neoplasms overexpressing a variety of cell surface GPCRs are actively targeted with the NPDCs described herein, thereby selectively delivering anti-cancer drugs or radionucliudes to the malignant cells.

GPCRs are a large and diverse group of integral membrane receptors and as a consequence are expressed in every cell type in the body. The function of GPCRs is to detect a host of signals outside the cell including but not limited to light, peptides, lipids, sugars and proteins and transmit the signal across the membrane to convert into intracellular responses. Because of these critical actions, the GPCR superfamily is the largest and most important family of drug targets as highlighted by the large number of approved therapeutics targeting this class. GPCRs are generally poorly antigenic making them difficult targets for antibody-based strategies. For many GPCRs, a large proportion of the protein population resides in intracellular compartments at any given time reducing the total number of cell surface binding sites accessible to antibodies or peptides.

Many GPCRs, especially those that recognize endogenous peptides and endogenous proteins, such as chemokines, are ideal for NPDCs with suitable drug cargos or payloads because of their restricted physiologic expression and frequent overexpression in particularly intractable cancers (Reubi et al, The Journal Of Nuclear Medicine, Vol. 58, No. 9 (Suppl. 2), 10S-15S). Many human tumors overexpress different GPCRs, often times at significantly higher density than other tissues. For example, gastroenteropancreatic (GEP) neuroendocrine tumors (NETs) overexpress somatostatin receptors, namely SSTR2, SSTR3, and SSTR5. Other peptide receptors are overexpressed in NETs, such as the incretin receptor glucagonlike peptide 1 (GLP-1), the glucose-dependent insulinotropic polypeptide (GIP) receptor, and cholecystokinin (CCK) receptors (CCK1 and CCK2 subtypes). Medullary thyroid carcinomas (MTC) overexpress the CCK2 receptor and GIP receptors. Breast cancers overexpress gastrin-releasing peptide (GRP) receptors, Y1 subtype of neuropeptide Y (NPY) receptors, SSTR2, and CXCR4. Due to the complicated GPCR overexpression profiles in neoplasms, targeting multiple receptors simultaneously may address issues such as heterogeneity, resistance, and change of phenotype during disease progression that have hampered many current treatment options.

Most currently available GPCR-targeting drugs act at receptors for which the native ligands are small molecules, such as histamine, adrenaline, and neurotransmitters. Drugs targeting GPCR for which the native ligands are peptides or proteins, are typically also peptides or proteins.

Peptides are intrinsically sensitive to proteolytic enzymes and peptidases present in most tissues, and are rapidly degraded into multiple fragments which no longer have significant affinity to the intended receptors. There are ways to stabilize peptides (e.g. incorporating peptidomimetic structures or using more stable D amino acids in the peptide backbone) but such variations may lead to loss of affinity and/or selectivity, and negatively impact physicochemical properties (e.g. poor solubility and tendency to aggregate). In addition, peptides may cause unwanted immunogenic responses complicating later stages of development by masking the therapeutic effect and impacting the safety assessment.

When peptide ligands are linked to cytotoxic payloads, the resulting conjugates often degrade rapidly in blood plasma and produce cytotoxic peptide fragments which may nonspecifically bind to both tumor and normal tissue. Such premature breakdown of peptide drug conjugates (PDCs) and antibody drug conjugates (ADCs) reduce the amount of cytotoxic payloads distributed to targeted tumors, lowering treatment efficacy, and possibly increasing toxicity. In addition, peptides are most likely exclusively excreted via kidney, which may limit their applications. Marked kidney uptake of some peptide-based therapeutics has limited their routine use.

High affinity, small molecule ligands that bind peptide GPCRs and protein GPCRs, such as chemokine GPCRs, have been described and are cell permeable and can access populations of receptors in endoplasmic reticulum and endosomes. Owing to the low molecular weight of non-peptide small molecules, vascular permeability and tumor penetration should be improved compared to high molecular weight conjugates based on peptides and antibodies. The binding affinity of small molecule nonpeptide ligands in many cases surpasses that of FDA approved antibodies by orders of magnitude.

Provided herein are non-peptide drug conjugates (NPDCs). When the non-peptide ligand of a NPDC, which is a small molecule, binds to a GPCR, it embeds in the extracellular loops of the GPCR, leaving the conjugated drug cargo or payload moiety of the NPDC pointing to extracellular space. The conjugated drug cargo or payload moiety is linked to the non-peptide ligand in a manner that does not affect the binding affinity of the nonpeptide ligand to the GPCR. The conjugated drug cargos or payload moieties include chemotherapeutic agents, which are linked to the nonpeptide ligand using a stable linker or cleavable linker.

In some embodiments, slowly dissociating nonpeptide ligands for a GPCR can maintain therapeutically effective concentrations in a target tissue long after they have been cleared from the systemic circulation resulting in improved therapeutic windows and prolonged duration of action compared to their circulating plasma concentrations. In some embodiments, similar optimization of receptor residence time may be used as a selective tumor targeting mechanism to increase local concentrations or prolong tumor residence times of toxin conjugates in tumors, without relying on tumor specific intracellular trafficking. This same principle of concentration of GPCR ligands in target tissues enables PET labeling or radioligand imaging studies to visualize defined regions of receptor expression. If the conjugates are stably linked, release of nonselective free toxin can be avoided and conjugates that initially “miss” their cytotoxic target can be retained in the tumor for additional attempts. In some embodiments, coupling of suitable payloads (e.g. toxins) to nonpeptide small molecule GPCR ligands provides an improved method for targeting of cytotoxic conjugates to cancerous tissues.

In some embodiments, nonpeptide ligands for a GPCR that are internalized are optimized to increase internalization and improve intracellular retention.

### Nonpeptide Small Molecule Drug Conjugates (NPDCs)

In one aspect, described herein is a non-peptide drug conjugate (NPDC). In some embodiments, the NPDC is a compound having the structure of Formula (I), or a pharmaceutically acceptable salt thereof:

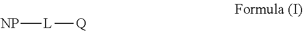

wherein:
(a) NP is a non-peptide ligand that binds to a G protein-coupled receptor (GPCR) expressed in tumor cells;
(b) a payload moiety (Q) comprising a chemotherapeutic; and
(c) a linker (L) that covalently connects the non-peptide small molecule ligand NP and the payload moiety Q;
wherein the linker L is attached to NP at a position that permits binding of NP to the peptide or protein GPCR; and wherein upon administration to a mammal with cancer, the compound of Formula (I), or a pharmaceutically acceptable salt thereof, is targeted to the tissues comprising tumor cells expressing the GPCR.

### Targeted Tissues and Receptors

In some embodiments, the compound of Formula (I) demonstrates activity for the targeted GPCR receptor. In some embodiments, the activity is functional activity. In some embodiments, the activity is binding affinity. In some embodiments, the compound of Formula (I) demonstrates functional activity for the targeted GPCR receptor. In some embodiments, the compound of Formula (I) demonstrates binding affinity for the targeted GPCR receptor. In some embodiments, the compound of Formula (I) demonstrates binding affinity or functional activity to the targeted GPCR receptor with a binding affinity or functional activity that is less than 100 nM as measured in a suitable in vitro assay that measures such binding activity or functional activity. In some embodiments, the compound of Formula (I) demonstrates binding affinity or functional activity to the targeted GPCR receptor with a binding affinity or functional activity that is less than 100 nM as measured in a suitable in vitro assay that measures such binding activity or functional activity. In some embodiments, the compound of Formula (I) demonstrates binding affinity or functional activity to the targeted GPCR receptor with a binding affinity that is less than 10 nM as measured in a suitable in vitro assay that measures such binding activity. In some embodiments, the compound of Formula (I) demonstrates binding affinity or functional activity to the targeted GPCR receptor with a binding affinity or functional activity that is less than 5 nM as measured in a suitable in vitro assay that measures such binding activity. In some embodiments, the compound of Formula (I) demonstrates binding affinity or functional activity to the targeted GPCR receptor with a binding affinity or functional activity that is less than 1 nM as measured in a suitable in vitro assay that measures such binding activity or functional activity.

In some embodiments, the compound of Formula (I) has a binding affinity or functional activity to the targeted GPCR receptor that is at least 10-fold, at least 50-fold, at least 100-fold, at least 200-fold, at least 500-fold, or at least 1000-fold greater than the binding affinity or functional activity for non-target receptors. In some embodiments, the compound of Formula (I) is selective for one GPCR. In some embodiments, the compound of Formula (I) is selective for one family of GPCRs (e.g., the somatostatin family of receptors, which include SSTR1, SSTR2, SSTR3, SSTR4, SSTR5). In some embodiments, the compound of Formula (I) is selective for one GPCR within a family of GPCRs. In some embodiments, the compound of Formula (I) has a binding affinity or functional activity for a GPCR that is at least 10-fold greater than its binding affinity or functional activity for any other GPCR. In some embodiments, the compound of Formula (I) has a binding affinity or functional activity for one or more GPCRs.

In some embodiments, the compound of Formula (I) is stable in the presence of liver microsomal enzymes. In some embodiments, the compound of Formula (I) is stable in the presence of proteases. In some embodiments, the compound of Formula (I) is stable in blood plasma. In some embodiments, the compound of Formula (I) is stable in blood plasma and optionally internalized into tumor cells after binding to the GPCR expressed in tumor cells. In some embodiments, the compound of Formula (I) comprises an optional cleavable linker L, is stable in blood plasma, optionally internalized into tumor cells after binding to the cancer cell surface peptide GPCR or protein GPCR, and the linker L is cleaved to intracellularly to release the payload or drug cargo moiety moiety (Q). In some embodiments, the linker L facilitates a controlled release of the payload or drug cargo moiety moiety (Q) inside tumor cells.

In some embodiments, the compound of Formula (I) preferentially accumulates in tumor tissues that express the targetted GPCR. In some embodiments, the compound of Formula (I) preferentially accumulates in tissues or organs comprising tumor cells that express the GPCR(s) as compared to tissues or organ(s) lacking tumor cells that express the GPCR(s). In some embodiments, the compound of Formula (I) preferentially accumulates at least 1-fold, at least 2-fold, 3-fold, at least 4-fold, at least 5-fold, or greater than 5-fold more in tissues or organ(s) comprising tumor cells that express the GPCR(s) as compared to tissues or organs lacking tumor cells that express the GPCR(s). It is understood that the compound may accumulate in certain tissues and organs involved in the metabolism and/or excretion of therapeutics, including but not limited to the kidneys and liver.

In some embodiments, the GPCR targeted by the compound of Formula (I) is expressed at higher levels and/or at higher concentrations in or by tumor cells and at substantially lower levels in or by non-tumor cells.

In some embodiments, the GPCR targeted by the compound of Formula (I) is expressed in tumor cells in tissues and/or organs that normally do not express the GPCR.

In some embodiments, NP binds to a GPCR expressed in a tumor cell, provided that NP does not comprise an unnatural amino acid residue that is 2-amino-2-adamantane carboxylic acid, cyclohexylglycine, or 9-amino-bicyclo[3.3.1]nonane-9-carboxylic acid.

In some embodiments, the GPCR is a receptor for an endogenous peptide or an endogenous protein ligand, wherein endogenous peptide or endogenous protein ligand is a peptide or protein hormone or a chemokine.

### Peptide GPCRs

Peptide hormones play regulatory functions mainly in the brain, gut, and endocrine system. These peptides are important in biology, but their receptors have become increasingly relevant clinically because they are often overexpressed in malignant tumors. In many instances, these peptide receptors are overexpressed in cancer cells, in comparison to their expression in normal tissue adjacent to the neoplasm and/or in its normal tissue of origin. The different level of receptor expression allows high uptake of peptide hormone receptor ligand conjugates in tumor cells, while none or low uptake of such conjugates in cells do not express receptors. Thus, cytotoxic payload attached as part of the conjugate can kill tumor cells while cause minimum toxicity. This feature allows peptide hormone ligand radionuclide conjugate to perform receptor-targeted imaging and therapy.

Numerous hormone-activated GPCRs are overexpressed in hormone-dependent and independent tumors and trigger multiple transduction pathways, which mediate relevant biological effects in diverse cancer cells. For example, bradykinin (BK) receptors are overexpressed in prostate cancer and mediate cell growth through Gαq and/or Gα13 which activate RhoA-dependent signaling.

Among the GPCR family members, the gonadotropin-releasing hormone (GnRH) receptor is a well-established target in the clinical practice of cancer treatment. GnRH receptors are expressed not only in the pituitary and in normal peripheral tissues, but also in various tumor cells like melanoma, prostate and endometrial carcinomas, leiomyomas, leiomyosarcomas, breast cancer, choriocarcinoma, epithelial and stromal tumors of the ovary. Several human tumor types, including ovarian, prostate, breast, and lung cancer, overexpress or even uniquely express this receptor with respect to the surrounding nonmalignant cells. GnRH analogues carrying cytotoxic agents directly to cancer cells expressing GnRH receptors has been described (e.g. a conjugate made from doxorubicin linked to [D-Lys6]GnRH agonist) and shown to reduce the proliferation of breast, ovarian and endometrial cancer cells in vitro and in xenografted nude mice.

The class of somatostatin receptors (SSTRs) consists of five members (SSTR1-5), which are widely expressed in different tissues in the body including nervous, pituitary, kidney, lung, and immune cells. Their natural ligand is the neuropeptide somatostatin (SST), which occurs in two active isoforms, the SST-14 and SST-28. In combination with their receptors, both isoforms act as inhibitory hormones. An important physiological function of the SSTR/SST axis is, for example, the inhibition of the release of growth hormones. SSTRs, particularly SSTR subtype 2, are found highly expressed in many neoplastic cells and in tumoral blood vessels. Overexpression of SSTRs and in particular SSTR2 has been found in various neuroendocrine tumors, as well as other tumors such as breast, ovarian, and lung cancer. Targeting of the SSTR2 for drug delivery has been accomplished by using stabilized, cyclic somatostatin analogs such as octreotate, octreotide, and lanreotide. For example, covalently attaching a DOTA chelator to octreotide (DOTA-TATE, also known as DOTA-(Tyr³)-octreotate) has made it possible to target delivery of radionuclides to tumor cells expressing somatostatin receptors. ¹⁷⁷Lu DOTA-TATE therapy is a form of peptide receptor radionuclide therapy (PRRT) which targets somatostatin receptors and is a form of targeted drug delivery.

The bombesin (Bn) receptor family consists of three members, namely the BB1, BB2, and BB3 receptor, which are expressed in the central nervous system (CNS), but also in the periphery such as the gastrointestinal tract. They mediate a multitude of physiological functions, including an autocrine growth action on cells and potent CNS effects. The natural peptide ligand for the BB1 is the neuromedin B and for the BB2 the gastrin-releasing peptide, while the BB3 is considered an orphan receptor. Upregulation of Bn receptors was found in various cancer subtypes and especially the BB2 is highly overexpressed in tumors such as breast, prostate, small cell lung, and pancreatic cancer. Targeting the Bn receptors for drug delivery has typically centered on the use of Bn analogs, including for example the peptide [d-Tyr6,β-Ala11,Phe13,Nle14]-Bn(6-14).

The vasoactive intestinal peptide (VIP) receptors 1 and 2, which are overexpressed in various cancers such as colon, breast, and endocrine tumors. The natural ligand VIP and its analogs are investigated for the preparation of drug conjugates.

The cholecystokinin 2 receptor (CCK2R) is overexpressed in various cancers of the thyroid, lung, pancreas, liver, and the gastrointestinal tract. Targeting of this receptor for drug delivery has typically concerned the use of analogs of its natural peptide ligands cholecystokinin and gastrin.

The melanocortin receptor 1 (MC1R) was found to be upregulated in malignant melanoma. For the generation of drug conjugates for this system, shortened peptide analogs of the natural MC1R ligand α-MSH, for example, the agonist NAPamide, possess the potential as delivery agents.

The ghrelin receptor (GhrR), also named growth hormone secretagogue receptor 1a (GHSR1α), is a class A GPCR. It is widely expressed in the brain, especially in the hypothalamus, but also in the hippocampus and the pituitary. Furthermore, the GhrR was found to be expressed in a variety of peripheral tissues, including liver, heart, pancreas, thyroid, ovaries, testis, and more. The natural ligand of the GhrR is the peptide hormone ghrelin, a 28-amino acid peptide. Ligand binding to the GhrR occurs rather deep in the cavity created by the TM helices of the receptor. The ghrelin/GhrR axis plays a role for a multitude of physiological functions such as food intake, regulation of energy homeostasis, release of various hormones (e.g., growth hormone, prolactin, adrenocorticotropic hormone) and reward-seeking behavior. The GhrR is present in a vast number of different cancer subtypes. Expression of the GhrR was described in pituitary adenomas, thyroid, breast, lung, testis, ovarian, prostate, pancreatic, gastric, and colorectal cancer, as well as in astrocytoma.

The human Y1 receptor (hY1R) is a class A GPCR from the Y receptor family in human and is predominantly expressed in the CNS, for example, the hypothalamus, but also found in peripheral tissues including heart, lung, or smooth muscle. Besides the hY1R, three other Y receptors are expressed in human, namely the Y2 receptor (hY2R), the Y4 receptor (hY4R), and the Y5 receptor (hY5R). These receptors are bound and activated by the neuropeptide Y family of peptide hormones, which consists of the neuropeptide Y (NPY), the peptide YY (PYY), and the human pancreatic polypeptide (hPP). NPY was found to be the most abundant peptide hormone in the mammalian CNS. Endogenous NPY is a 36-amino acid peptide and consists of a flexible N-terminus, a C-terminal amphipathic α-helix, and an amidated C-terminus. The presence in certain tumor tissues renders the hY1R a target for anti-cancer drug delivery. Expression of the hY1R together with the hY2R has been described in ovarian sex cord-stromal tumors, nephroblastomas, gastrointestinal stromal tumors, and testicular tumors. Sole expression of the hY1R was observed in adrenal cortical tumors and renal cell carcinomas. High expression of the hY1R was also determined in Ewing sarcoma tumors and breast cancer tumors and breast cancer-derived metastases. In contrast, in the surrounding non-neoplastic breast tissue expression of the hY2R was predominantly observed. This switch in the Y receptor expression pattern during neoplastic transformation of breast tissue therefore enables a specific drug shuttling into breast tumors when using a hY1R-preferring ligand as delivery agent.

Oprhan GPCRs have been linked to cancer development and progression on the basis of their overexpression and/or up-regulation by diverse factors. For instance, an elevated expression of the orphan G-protein-coupled receptor GPR49 was involved in the formation and proliferation of basal cell carcinoma, while GPR18 was found associated with melanoma metastases. In lung, cervix, skin, urinary bladder, testis, head and neck squamous cell carcinomas were detected high levels of GPR87.

As used herein, “peptide G protein-coupled receptor (GPCR)” means a GPCR that is the binding site for a peptide ligand. The native ligand for a peptide GPCR is a peptide ligand.

As used herein, “protein G protein-coupled receptor (GPCR)” means a GPCR that is the binding site for a protein ligand. The native ligand for a protein GPCR is a protein ligand.

In some embodiments, NP of the compound of Formula (I) binds to a GPCR that also binds to a peptide or protein hormone that is: adrenocorticotropic hormone (ACTH), amylin, angiotensin, atrial natriuretic peptide (ANP), calcitonin, cholecystokinin (CCK), gastrin, ghrelin, glucagon, growth hormone, follicle-stimulating hormone (FSH), insulin, leptin, melanocyte-stimulating hormone (MSH), oxytocin, parathyroid hormone (PTH), prolactin, renin, somatostatin, thyroid-stimulating hormone (TSH), thyrotropin-releasing hormone (TRH), vasopressin, or vasoactive intestinal peptide.

In some embodiments, NP binds to a GPCR, provided that the GPCR does not bind neurotensin.

In some embodiments, the GPCR is: angiotensin receptor; apelin receptor; bombesin receptor; bradykinin receptor; calcitonin receptor; chemokine receptor; cholecytokinin receptor; corticotropic-releasing factor receptor; galanin receptor; ghrelin receptor; glucagon receptor; glycoprotein hormone receptor; gonadotropin-releasing hormone receptor; kisspeptin receptor; melanocortin receptor; motilin receptor; neuromedin U receptor; neuropeptide FF/AF receptor; neuropeptide S receptor; neuropeptide W/B receptor; neuropeptide Y receptor; opioid receptor; orexin receptor; parathyroid hormone receptor; prokineticin receptor; prolactin-releasing peptide receptor; QRFP receptor; relaxin family peptide receptor; somatostatin receptor; tachykinin receptor; thyrotropin-releasing hormone receptor; urotensin receptor; vasopressin and oxytocin receptor; VIP and PACAP receptor; or combinations thereof.

In some embodiments, the GPCR is a member of one of the following families of receptors: angiotensin receptors (e.g. AGTR1, AGTR2); apelin receptor (APLNR); bombesin receptors (BB1/NMBR, BB2/GRPR, BRS3); bradykinin receptors (BDKRB1, BDKRB2); calcitonin receptor (e.g. CALCR, CALCRL); chemokine receptors (e.g. CCR1-10, CXCR1-6, etc); cholecytokinin receptors (CCKAR, CCKBR); corticotropic-releasing factor receptors (CRHR1, CRHR2); galanin receptors (GALR1-3); ghrelin receptor (GHSR); glucagon receptor family (e.g. GHRHR, GIPR, GLP1R, GLP2R, GCGR, SCTR); glycoprotein hormone receptors (FSHR, LHCGR, TSHR); gonadotropin-releasing hormone receptor (GNRHR, GNRHR2); kisspeptin receptor (KISSIR); melanocortin receptors (MC₁₋₅R); motilin receptor (MLNR); neuromedin U receptors (NMUR1, NMUR1-2); neuropeptide FF/AF receptors (NPFFR1, NPFFR2); neuropeptide S receptors (NPSR1); neuropeptide W/B receptors (NPBWR1, NPBWR2); neuropeptide Y receptors (NPY1-6R); opioid receptors (OPRD1, OPRK1, OPRM1); orexin receptors (HCRTR1, HCRTR2); parathyroid hormone receptors (PTH1R, PTH2R); prokineticin receptors (PROKR1, PROKR2); prolactin-releasing peptide receptor (PRLHR); QRFP receptor (QRFPR); relaxin family peptide receptors (RXFP1-4); somatostatin receptors (SSTR1-5); tachykinin receptors (TACR1-3); thyrotropin-releasing hormone receptor (TRHR1, TRHR2); urotensin receptor (UTS2R); vasopressin and oxytocin receptors (AVPR1A, AVPR1B, AVPR2, OXTR); or VIP and PACAP receptors (ADCYAP1R1, VIPR1, VIPR2).

In some embodiments, the GPCR is a member of one of the following families of receptors: angiotensin receptors (e.g. AGTR1, AGTR2); apelin receptor (APLNR); bombesin receptors (BB1/NMBR, BB2/GRPR); bradykinin receptors (BDKRB1, BDKRB2); ghrelin receptor (GHSR); glycoprotein hormone receptors (FSHR, LHCGR); gonadotropin-releasing hormone receptor (GNRHR); kisspeptin receptor (KISSIR) melanocortin receptor family (MC1R, MC2R, MC3R, MC4R, MC5R); neuropeptide Y receptors (NPY1); neurotensin receptors (NTSR1); parathyroid hormone receptors (PTH1R); prolactin-releasing peptide receptor (PRLHR); somatostatin receptor family (SSTR1, SSTR2, SSTR2, SSTR4, SSTR5); thyrotropin-releasing hormone receptor (TRHR1, TRHR2); or VIP and PACAP receptors (ADCYAP1R1, VIPR1, VIPR2).

### Chemoattractant GPCRs

In some embodiments, the GPCR is a chemoattractant GPCR. In some embodiments, the GPCR is a chemoattractant GPCR that is: a classical GPCR that is formyl peptide receptor (FPR1, FPR2, or FPR3), platelet activating factor receptor (PAFR), activated complement component 5a receptor (C₅aR); or a chemokine GPCR that is binds to a CC chemokine (0-chemokine), CXC chemokine (α-chemokine), C chemokine (γ chemokine), or CX3C chemokine (d-chemokine).

Tumor cells that metastasize preferentially to specific organs via the blood and lymphatic vessels present a great challenge in cancer eradication. One family of GPCRs that is closely linked to tumor metastasis the chemokine receptors. Chemokines enhance the motility and survival of cancer cells in the vicinity and milieu of a tumor following their local release in either an autocrine or paracrine fashion into the microenvironment of tumor-surrounding regions. Among these are chemokines that are involved in metastatic cancer cell homing as well as cancer cell growth and survival, such as chemokine receptors CCR7 and CCR10. Local chemokine generation in the tumor milieu may recruit macrophages and leukocytes, which can then induce the release of matrix metalloproteases (MMPs) promoting tumor cell survival, growth, and invasion as well as improving the cytokine-rich microenvironment. CXCR4 is a well-documented chemokine receptor driving cancer metastasis. Moreover, cells in the most frequent sites of metastasis, including the lungs, bone marrow, lymph nodes, and liver, express the chemokine ligand CXCL12/SDF-1.

Tumor cells frequently express high levels of CXCR4, facilitating cell growth, survival, and migratory capability. For example, while CXCR4 is not found in normal breast tissues, it is rather overexpressed in breast cancer cells and a marked inhibition of breast cancer metastatic spread is achieved by inhibiting CXCR4. However, treatment with CXCR4 inhibitors requires caution, since CXCR4 inhibition induces progenitor/stem cell mobilization from the bone marrow. Hypoxia-inducible factor-1 (HIF-1α), which is activated by hypoxia, increases CXCR4 transcription. In highly aggressive basal-like breast cancer cells, CXCR4 may also couple to Gα12/13 when Gα13 protein is highly upregulated, and consequently drives spread via lymphatic vessels and site-specific metastasis in a Gα12/13-RhoA-dependent manner. This molecular machinery is mediated similarly via PARs and LPA, all of which may serve as possible targets for metastasis prevention and treatment.

### Chemokine GPCRs

| Receptor | Expression | Function |
|---|---|---|
| CCR1 | Monocytes, neutrophils, T lymphocytes, | Chemotaxis and activation |
| | dendritic cells, tumor cells | Tumor growth, metastasis, angiogenesis |
| CCR2 | Monocytes, basophils, T lymphocytes, | Chemotaxis and activation |
| | dendritic cells, NK cells, endothelial | Tumor growth, metastasis, angiogenesis |
| | cells, tumor cells | |
| CCR3 | Eosinophils, basophils, Th2 | Chemotaxis and activation |
| | lymphocytes, mast cells, tumor cells | Tumor growth, metastasis |
| CCR4 | Macrophages, monocytes, basophils, T | Chemotaxis and activation |
| | and B lymphocytes, dendritic cells, NK | Tumor growth, metastasis, angiogenesis |
| | cells, mast cells, platelets, tumor cells | |
| CCR5 | Macrophages, T lymphocytes, dendritic | Chemotaxis and activation |
| | cells, NK cells, tumor cells | Tumor growth, metastasis, |
| | | angiogenesis |
| CCR6 | Neutrophils, T and B lymphocytes, | Chemotaxis and activation |
| | dendritic cells, epithelial cells of some | Tumor growth, metastasis |
| | tissues, tumor cells | |
| CCR7 | T and B lymphocytes, dendritic cells | Lymphoid tissue chemotaxis and activation |
| | Tumor cells | Tumor growth, metastasis |
| CCR8 | Macrophages, Th2 lymphocytes, | Chemotaxis and activation |
| | endothelial cells | Tumor metastasis |
| | Tumor cells | |
| CCR9 | T lymphocytes | Small intestinal specific chemotaxis and |
| | Tumor cells | activation; tumor growth, metastasis; |
| | | inhibiting tumor metastasis in some tumors |
| CCR10 | T lymphocytes | Skin-specific chemotaxis and activation |
| | Tumor cells | Tumor growth, metastasis, angiogenesis |
| CXCR1 | Neutrophils, polymorphonuclear | Chemotaxis and activation Tumor growth, |
| | leukocytes, endothelial cells Tumor cells | metastasis, angiogenesis |
| CXCR2 | Neutrophils, basophils, T lymphocytes, | Chemotaxis and activation Tumor growth, |
| | oligodendrocytes, endothelial cells | metastasis, angiogenesis |
| | Tumor cells | |
| CXCR3 | Macrophages, T lymphocytes, NK cells, | Chemotaxis and activation |
| | NKT cells, endothelial cells Tumor cells | Two variants CXCR3-A and CXCR3-B |
| | | have opposite function in tumor |
| | | progression |
| CXCR4 | Numerous cell types: hematopoietic | Chemotaxis and activation Maintenance of |
| | cells and stem cells | stem phenotype Tumor growth, metastasis, |
| | Tumor cells | angiogenesis |
| CXCR5 | T and B lymphocytes | Chemotaxis and activation |
| | Tumor cells | Tumor growth, metastasis; inhibiting |
| | | tumor metastasis in some tumors |
| CXCR6 | T and B lymphocytes, NK cells, NKT | Chemotaxis and activation Tumor growth, |
| | cells, plasma cells | metastasis, angiogenesis; inhibiting tumor |
| | Tumor cells | migration in some tumors |
| CXCR7 | T and B lymphocytes, dendritic cells, | Chemotaxis and activation Tumor growth, |
| | endothelial cells, fetal hepatocytes | metastasis, angiogenesis; assisting with |
| | Tumor cells | CXCR4 to regulate tumor progression |
| CX3CR1 | Monocytes, T and B lymphocytes, mast | Chemotaxis and activation |
| | cells, dendritic cells, NK cells Tumor | Tumor growth, metastasis; inhibiting |
| | cells | tumor invasion in some tumors |
| XCR1 | Neutrophils, T lymphocytes, dendritic | Chemotaxis and activation |
| | cells; tumor cells | |

Solid Tumors: Benign and/or Malignant Neoplasms (Cancer)

In one aspect, compounds of Formula (I) are used to treat benign and/or malignant neoplasms (solid tumors), wherein the neoplasm comprises cells that overexpress cell surface GPCRs.

The term “neoplasm” as used herein, refers to an abnormal growth of cells that may proliferate in an uncontrolled way and may have the ability to metastasize (spread).

Neoplasms include solid tumors, adenomas, carcinomas, sarcomas, leukemias and lymphomas, at any stage of the disease with or without metastases.

A solid tumor is an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors.

Solid tumors are cancers that typically originate in organs, such as the bladder, bowel, brain, breast, endometrium, heart, kidney, lung, liver, uterus, ovaries, pancreas or other endocrine organs (thyroid), and prostate.

An adenoma is a tumor that is not cancer. It starts in gland-like cells of the epithelial tissue (thin layer of tissue that covers organs, glands, and other structures within the body). An adenoma can grow from many glandular organs, including the adrenal glands, pituitary gland, thyroid, prostate, and others. Over time adenomas may transform to become malignant, at which point they are called adenocarcinomas. Even though benign, they have the potential to cause serious health complications by compressing other structures (mass effect) and by producing large amounts of hormones in an unregulated, non-feedback-dependent manner (causing paraneoplastic syndromes).

Adenomas typically are found in the colon (e.g. adenomatous polyps, which have a tendency to become malignant and to lead to colon cancer), kidneys (e.g. renal adenomas may be precursor lesions to renal carcinomas), adrenal glands (e.g. adrenal adenomas; some secrete hormones such as cortisol, causing Cushing's syndrome, aldosterone causing Conn's syndrome, or androgens causing hyperandrogenism), thyroid (e.g. thyroid adenoma), pituitary (e.g. pituitary adenomas, such as prolactinoma), parathyroid (e.g. an adenoma of a parathyroid gland may secrete inappropriately high amounts of parathyroid hormone and thereby cause primary hyperparathyroidism), liver (e.g. hepatocellular adenoma), breast (e.g. fibroadenomas), appendix (e.g. cystadenoma), bronchial (e.g. bronchial adenomas may cause carcinoid syndrome, a type of paraneoplastic syndrome), prostate (e.g. prostate adenoma), sebaceous gland (e.g. sebaceous adenoma), and salivary glands.

Metastasis the spread of malignant cells to new areas of the body, often by way of the lymph system or bloodstream. A metastatic tumor is one that has spread from the primary site of origin, or where it started, into different areas of the body. Metastatic tumors comprise malignant cells that express cell surface GPCRs.

Tumors formed from cells that have spread are called secondary tumors. Tumors may have spread to areas near the primary site, called regional metastasis, or to parts of the body that are farther away, called distant metastasis.

In some embodiments, the tumor to be treated comprises tumor cells expressing a GPCR, wherein the tumor is a primary or metastatic tumor. In some embodiments, the tumor to be treated comprises tumor cells expressing a GPCR, wherein the tumor is a primary or metastatic tumor of gastrointestinal origin, such as colorectal cancer, stomach cancer, small intestine cancer, or esophageal cancer. In some embodiments, the tumor to be treated comprises tumor cells expressing a GPCR, wherein the tumor is a primary or metastatic tumor of the pancreas. In some embodiments, the tumor to be treated comprises tumor cells expressing a GPCR, wherein the tumor is a primary or metastatic tumor of the lungs, such as squamous cell carcinoma, adenosquamous carcinoma, or adenocarcinoma. In some embodiments, the tumor to be treated comprises tumor cells expressing a GPCR, wherein the tumor is a primary or metastatic neuroectodermal tumor, such as aphaechromotcytoma or a paraganglioma. In some embodiments, the tumor to be treated comprises tumor cells expressing a GPCR, wherein the tumor is a primary or metastatic bronchopulmonary or gastrointestinal neuroendocrine tumor. In some embodiments, the tumor to be treated comprises tumor cells expressing a GPCR, wherein the tumor is a primary or metastatic tumor of the rectum or colon.

In some embodiments, compounds of Formula (I) are used to treat a sarcoma, such as leiomyosarcoma or rhabdomyosarcoma.

In some embodiments, compounds of Formula (I) are used to treat an adenoma.

In another aspect, provided herein is a method for treating cancer in a mammal comprising administering to the mammal in need thereof, a non-peptide targeted therapeutic disclosed herein. In some embodiments, the cancer comprises tumor cells expressing one or more peptide hormone GPCRs. In some embodiments, the cancer comprises tumor cells that overexpress one or more GPCRs. In some embodiments, the cancer comprises a solid tumor. In some embodiments, the cancer comprises a sarcoma, carcinoma, or lymphoma. In some embodiments, the cancer comprises a neuroendocrine tumor. In some embodiments, the cancer comprises an insulinoma. In some embodiments, the cancer comprises peptide hormone GPCR-positive (e.g., somatostatin receptor-positive) gastroenteropancreatic neuroendocrine tumors (GEP-NETs).

In some embodiments, the compound of Formula (I) is administered to an oncology patient. In some embodiments, the oncology patient has been diagnosed with a carcinoma, sarcoma, primary tumor, metastatic tumor, solid tumor, non-solid tumor, blood tumor, leukemia or lymphoma.

Carcinomas include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc.

Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Solid tumors include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Benign solid tumors include adenomas.

Leukemias include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocyte leukemia, and hairy cell leukemia; and d) acute lymphoblastic leukemias (characterized by accumulation of lymphoblasts). Lymphomas include, but are not limited to, B-cell lymphomas (e.g., Burkitt's lymphoma); Hodgkin's lymphoma; and the like.

Primary and metastatic tumors include, e.g., lung cancer (including, but not limited to, lung adenocarcinoma, squamous cell carcinoma, large cell carcinoma, bronchioloalveolar carcinoma, non-small-cell carcinoma, small cell carcinoma, mesothelioma); breast cancer (including, but not limited to, ductal carcinoma, lobular carcinoma, inflammatory breast cancer, clear cell carcinoma, mucinous carcinoma); colorectal cancer (including, but not limited to, colon cancer, rectal cancer); anal cancer; pancreatic cancer (including, but not limited to, pancreatic adenocarcinoma, islet cell carcinoma, neuroendocrine tumors); prostate cancer; ovarian carcinoma (including, but not limited to, ovarian epithelial carcinoma or surface epithelial-stromal tumor including serous tumor, endometrioid tumor and mucinous cystadenocarcinoma, sex-cord-stromal tumor); liver and bile duct carcinoma (including, but not limited to, hepatocellular carcinoma, cholangiocarcinoma, hemangioma); esophageal carcinoma (including, but not limited to, esophageal adenocarcinoma and squamous cell carcinoma); non-Hodgkin's lymphoma; bladder carcinoma; carcinoma of the uterus (including, but not limited to, endometrial adenocarcinoma, uterine papillary serous carcinoma, uterine clear-cell carcinoma, uterine sarcomas and leiomyosarcomas, mixed mullerian tumors); glioma, glioblastoma, medulloblastoma, and other tumors of the brain; kidney cancers (including, but not limited to, renal cell carcinoma, clear cell carcinoma, Wilm's tumor); cancer of the head and neck (including, but not limited to, squamous cell carcinomas); cancer of the stomach (including, but not limited to, stomach adenocarcinoma, gastrointestinal stromal tumor); multiple myeloma; testicular cancer; germ cell tumor; neuroendocrine tumor; cervical cancer; carcinoids of the gastrointestinal tract, breast, and other organs; and signet ring cell carcinoma.

### Payload Moieties (Q) Comprising Cytotoxic Payloads/Drugs

In some embodiments, Q comprises a chemotherapeutic drug.

In some embodiments, NP is a non-peptide ligand that binds to a GPCR expressed in tumor cells of a solid tumor, adenoma, sarcoma, carcinoma, or lymphoma; Q comprises a chemotherapeutic that is a cytotoxic drug, kinase inhibitor or both; L is an optional non-cleavable linker or an optional cleavable linker that is cleaved in the vicinity of the GPCR expressed in tumor cells or within the tumor cells.

In some embodiments, the optional cleavable linker is a pH-sensitive linker, redox-sensitive linker, or an enzymatically cleavable linker, wherein the enzymatically cleavable linker is cleaved by an esterase, protease or peptidase that is expressed in tissues comprising the GPCR expressed in tumor cells.

In some embodiments, Q comprises a chemotherapeutic that is a ligand for an extracellular protein in the extracellular environment of the tumor cells expressing the GPCR.

In some embodiments, Q comprises a cytotoxic drug that is an antimitotic, DNA-damaging agent, transcriptional inhibitor or combination thereof.

In some embodiments, Q comprises a cytotoxic drug that is an antimitotic, and wherein the antimitotic is a maytansinoid, taxane, auristatin, alkaloid, tubulysin, or epothilone.

In some embodiments, Q comprises a cytotoxic drug that is a DNA-damaging agent, transcriptional inhibitor, or combination thereof, wherein the DNA-damaging agent, transcriptional inhibitor, or combination thereof is a DNA polymerase inhibitor, DNA replication inhibitor, topoisomerase inhibitor, cytotoxic antibiotic.

In some embodiments, Q comprises a kinase inhibitor that is an inhibitor of a cytoplasmic tyrosine kinase (CTK), a serine/threonine kinase (S/T Kinase), a lipid kinase (LK), or a receptor tyrosine kinase (RTK).

In some embodiments, Q comprises a cytotoxic drug that is an antimitotic, DNA-damaging agent, transcriptional inhibitor or combination thereof. In some embodiments, Q comprises a cytotoxic drug that is an antimitotic, and wherein the antimitotic is a maytansinoid, taxane, auristatin, alkaloid, tubulysin, or epothilone. In some embodiments, the maytansinoid is mertansine or emtansine; the taxane is paclitaxel or docetaxel; the alkaloid is ellipticine or a vinca alkaloid; the Epothilone is epothilone A, epothilone B, epothilone C, epothilone D, epothilone E, epothilone F, or utidelone; the auristatin is monomethyl auristatin E or monomethyl auristatin F.

In some embodiments, the vinca alkaloid is vinblastine, vincristine, vindesine, vinorelbine, or vinflunine.

In some embodiments, the DNA-damaging agent, transcriptional inhibitor, or combination thereof is a DNA polymerase inhibitor, DNA replication inhibitor, topoisomerase inhibitor, cytotoxic antibiotic.

In some embodiments, the DNA polymerase inhibitor is amikhelline, aphidicolin, cytarabine, vernolepin; the DNA replication inhibitor is altretamine, bleomycin, cytarabine, dacarbazine, dactinomycin, ellipticine, estramustine, mitobronitol, mitomycin, mitosene, pingyangmycin, plicamycin, procarbazine, or temozolomide; the topoisomerase inhibitor is irinotecan, topotecan, camptothecin, etoposide, doxorubicin, mitoxantrone, teniposide, novobiocin, merbarone, or aclarubicin; the cytotoxic antibiotic is an anthracycline, bleomycin, mitomycin, or actinomycin.

In some embodiments, the anthracycline is doxorubicin, daunorubicin, epirubicin, idarubicin, pirarubicin, aclarubicin, or mitoxantrone; the mitomycin is mitomycin A, mitomycin B, or mitomycin C.

In some embodiments, a cytotoxic drug is selected from actinomycin, aldesleukin, alemtuzumab, alitretinoin, altretamine, amsacrine, anastrozole, azacitidine, azathioprine, bevacizumab, bexarotene, bicalutamide, bleomycin, bortezomib, botulinum toxin, busulfan, capecitabine, carboplatin, carmustine, cetuximab, clorambucil, chloramphenicol, chlormethine hydrochloride, ciclosporin, cidofovir, cisplatin, cladribine, clofarabine, clorambucil, crisantaspase, cyclophosphamide, cytarabine, dacarbazine, dactinomycin, danazol, dasatinib, daunorubicin hydrochloride, decitabine, denileukin, dienostrol, diethylstilbestrol, dinoprostone, docetaxel, doxorubicin, dutasteride, epirubicin, estradiol, estramustine phosphate sodium, estrogen-progestin combinations, estrogens conjugated, estrogens esterified, estrone, estropipate, etoposide, exemestane, finasteride, floxuridine, fludarabine, fluorouracil, fluoxymesterone, flutamide, fulvestrant, ganciclovir, gemcitabine, ibritumomab tiuxetan, idarubicin, ifosfamide, imatinib mesilate, irinotecan hydrochloride, leflunomide, letrozole, leuprorelin acetate, lomustine, lymphoglobuline, medroxyprogesterone, megestrol, melphalan, mercaptopurine, mesena, methotrexate, methyltestosterone, mifepristone, mitomycin, mitotane, mitoxantrone hydrochloride, mycophenolate mofetil, nafarelin, natalizumab, nilutamide, oestrogen, oxaliplatin, oxytocin (including syntocinon and syntometrine), paclitaxel, pemetrexed disodium, pentamidine isethionate, pentostatin, perphosphamide, pipobroman, piritrexim isethionate, plicamycin, podoflilox, prednimustine, procarbazine, progesterone containing products, progestins, raloxifene, raltitrexed, ribavirin, rituximab, sirolimus, streptozocin, tacrolimus, tamoxifen, temozolomide, teniposide, testolactone, testosterone, thalidomide, thioguanine, thiotepa, thymoglobulin, tioguanine, topotecan, toremifene citrate, tositumomab, trastuzumab, treosulfan, tretinoin, trifluridine, trimetrexate glucuronate, triptorelin, uramustine, valganciclovir, valrubicin, vidarabine, vinblastine sulfate, vincristine, vindesine, vinorelbine tartrate, and zidovudine.

In some embodiments, Q comprises:

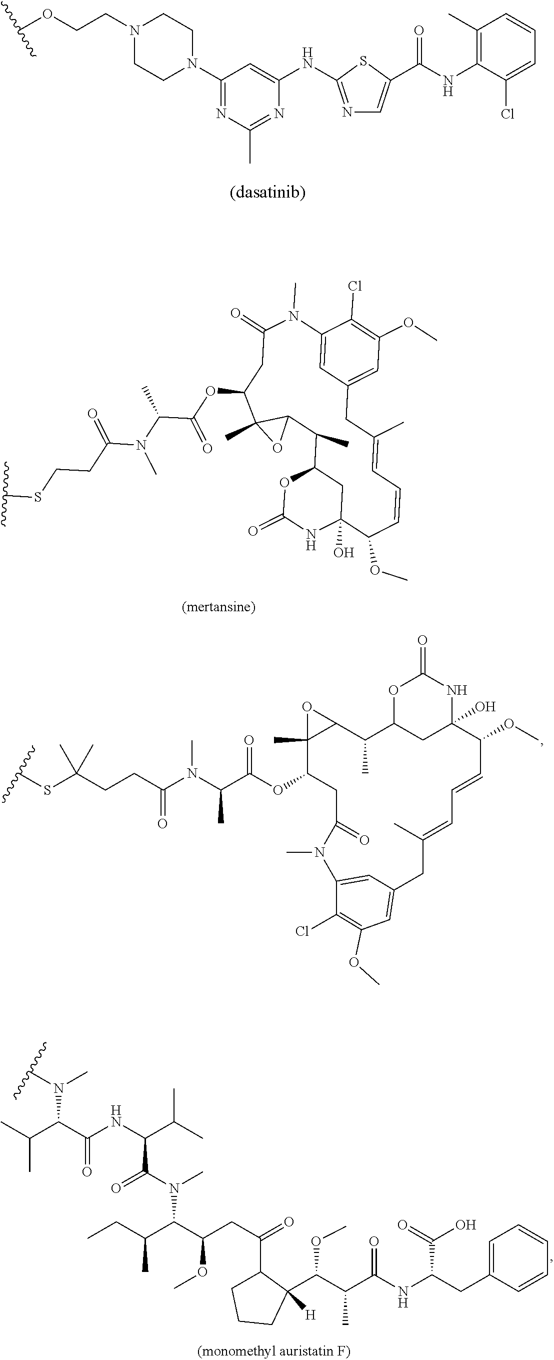

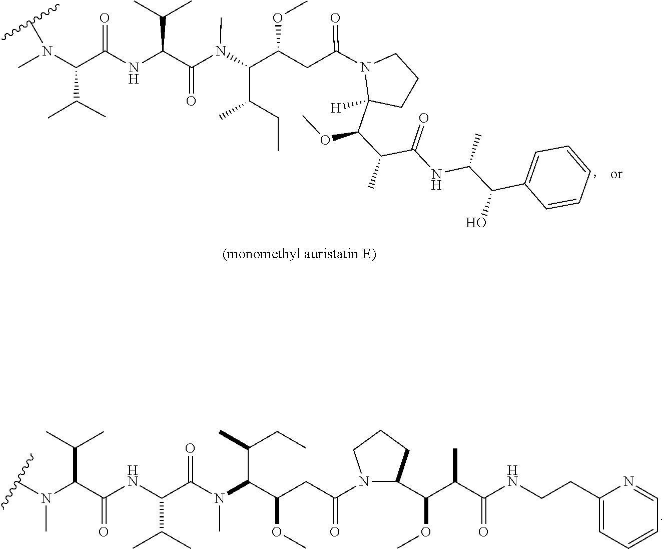

In some embodiments, Q comprises a kinase inhibitor. In some embodiments, Q comprises a kinase inhibitor that is an inhibitor of a cytoplasmic tyrosine kinase (CTK), a serine/threonine kinase (S/T Kinase), a lipid kinase (LK), or a receptor tyrosine kinase (RTK).

In some embodiments, the cytoplasmic tyrosine kinase (CTK) is Sphingosine kinase 1 (SK1), or phosphoinositide 3-kinase (PI3K).

In some embodiments, the serine/threonine kinase (S/T Kinase) is Protein kinase Ci (PKCi), mammalian target of rapamycin (mTOR), cyclin-dependent kinases (CDKs), Ataxia telangiectasia mutated (ATM), protein kinase B (Akt), ribosomal protein S6 kinase (S6K), Serine/threonine kinase 11 or liver kinase B1(STK11/LKB1), Polo-like kinases (PLKs), B-Raf proto-oncogene (b-Raf), Aurora Kinase A & B (Aur A & B), Mitogen Activated Protein (MAP) kinase kinase kinase (MAP3K), or Mitogen-activated protein kinase kinase (MEK);

In some embodiments, the lipid kinase (LK) is Proto-oncogene tyrosine-protein kinase Src (c-SRC), c-Yes proto-oncogene (c-YES), Abelson murine leukemia viral oncogene homolog 1 (Abl), or Janus kinase 2 (JAK-2).

In some embodiments, the receptor tyrosine kinase (RTK) is Recepteur d'Origine Nantais (RON), Fibroblast growth factor receptors (FGFRs), c-MET proto-oncogene (c-Met), c-RET proto-oncogene (c-Ret), Insulin-like growth factor 1 receptor (IGF-IR), Epidermal growth factor receptor (EGFR), Platelet-derived growth factor receptor α (PDGFR-α), proto-oncogene c-Kit or Mast/stem cell growth factor receptor (c-Kit), Fms-like tyrosine kinase 3, 4 (Flt3,Flt-4), Platelet-derived growth factor receptor β (PDGFR-β), Anaplastic lymphoma kinase (ALK), human epidermal growth factor receptor-2 (HER-2), or Bruton's tyrosine kinase (BTK).

In some embodiments, Q comprises acalabrutinib, afatinib, alectinib, axitinib, brigatinib, bosutinib, ceritinib, crizotinib, cabozantinib, dasatinib, dabrafenib, erlotinib, gefitinib, ibrutinib, imatinib, lapatinib, lenvatinib, nilotinib, nintedanib, osimertinib, palbociclib, pazopanib, ponatinib, regorafenib, ribociclib, ruxolitinib, sorafenib, sunitinib, trametinib, vandetanib, vemurafenib, or zanubrutinib.

### Linkers

In some embodiments, the linker has a prescribed length thereby linking NP and Q while allowing an appropriate distance therebetween.

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker comprises one or more linear structures, one or more non-linear structures, one or more branched structures, one or more cyclic structures, one or more flexible moieties, one or more rigid moieties, or combinations thereof.

In some embodiments, a linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids.

In some embodiments, the linker comprises a peptide linkage. The peptide linkage comprises L-amino acids and/or D-amino acids. In some embodiments, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, a linker has 1 to 100 atoms, 1 to 50 atoms, 1 to 30 atoms, 1 to 20 atoms, 1 to 15 atoms, 1 to 10 atoms, or 1 to 5 atoms in length. In some embodiments, the linker has 1 to 10 atoms in length. In some embodiments, the linker has 1 to 20 atoms in length.

In some embodiments, a linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences, proline-rich sequences, and regions rich in double and/or triple bonds.

In some embodiments, a linker is cleavable. In some embodiments, a linker is designed for cleavage in the presence of particular conditions or in a particular environment, such conditions or environments near such targeted cells, tissues, or regions. Cleavable linkers rely on the inherent properties of a cell's cytoplasmic compartments for selective release of the cytotoxic drug. Such linkers mainly include chemically cleavable linkers that respond to low pH (acid-labile linkers) or reducing environment (disulfide linkers), and enzymatically cleavable linkers that are susceptible to the action of certain lysosomal enzymes (peptide linkers or β-glucuronide linkers).

In some embodiments, a linker is cleavable under physiological conditions. In some embodiments, a linker is cleavable under intracellular conditions. In some embodiments, the linker is chemically cleavable. In some embodiments, the linker is enzymatically cleavable. In some embodiments, the linker is pH-sensitive, i.e., sensitive to hydrolysis at certain pH values. For example, the pH-sensitive linker can be hydrolyzable under acidic conditions. For example, a linker can be an acid-labile linker that is hydrolyzable in the lysosome (e.g., a hydrazone, semicarbazone, thiosemicarbazone, cis-aconitic amide, orthoester, acetal, ketal, or the like). Such linkers can be relatively stable under neutral pH conditions, such as those in the blood, but are unstable below pH 7.0, such as pH 6.5 to 4.5, the approximate pH of the lysosome and/or endosome.

In some embodiments, the linker comprises one or more of di-sulfide bonds.

In some embodiments, the linker is cleaved in or near tissues suffering from hypoxia, such as cancer cells and cancerous tissues. In some embodiments, the linker comprises a disulfide bond. In some embodiments, a linker comprising a disulfide bond is preferentially cleaved in hypoxic regions. Hypoxia is thought to cause cancer cells to become more resistant to radiation and chemotherapy, and also to initiate angiogenesis. In a hypoxic environment in the presence of, for example, leaky or necrotic cells, free thiols and other reducing agents become available extracellularly, while the O₂ that normally keeps the extracellular environment oxidizing is by definition depleted. In some embodiments, this shift in the redox balance promotes reduction and cleavage of a disulfide bond within a linker. In addition to disulfide linkages which take advantage of thiol-disulfide equilibria, linkages including quinones that fall apart when reduced to hydroquinones are used in a linker designed to be cleaved in a hypoxic environment.

In some embodiments, the linker is cleaved by an intracellular peptidase or protease enzyme, including, but not limited to, a lysosomal or endosomal protease. In some embodiments, the linker is cleaved by a glycosidase, e.g., glucuronidase. Small peptide sequences such as Val-Cit and Phe-Lys have been developed as linkers for ADCs. These bi-peptide linkers show good stability in serum, yet can be recognized and rapidly hydrolyzed by certain lysosomal proteases, such as cathepsin B, following internalization. β-glucuronide linkers can be readily cleaved by the abundant lysosomal enzyme β-glucuronidase, facilitating facile and selective release of the active drug. In other embodiments, the linker is not cleavable.

In some embodiments, the linker is cleaved by a protease, a matrix metalloproteinase, a serine protease, or a combination thereof. In some embodiments, the linker is cleaved by a reducing agent. In some embodiments, the linker is cleaved by an oxidizing agent or oxidative stress.

In some embodiments, the linker is cleaved by an MMP. The hydrolytic activity of matrix metalloproteinases (MMPs) has been implicated in the invasive migration of metastatic tumor cells. In some embodiments, a linker includes the amino-acid sequences PLG-C(Me)-AG, PLGLAG which are cleaved by the metalloproteinase enzymes MMP-2, MMP-9, or MMP-7 (MMPs involved in cancer and inflammation).

In some embodiments, the linker is cleaved by proteolytic enzymes or reducing environment, as may be found near cancerous cells. Such an environment, or such enzymes, are typically not found near normal cells.

In some embodiments, the linker is cleaved by serine proteases including but not limited to thrombin and cathepsins. In some embodiments, the linker is cleaved by cathepsin K, cathepsin S, cathepsin D, cathepsin E, cathepsin W, cathepsin F, cathepsin A, cathepsin C, cathepsin H, cathepsin Z, or any combinations thereof. In some embodiments, the linker is cleaved by cathepsin K and/or cathepsin S.

In some embodiments, the linker is cleaved in a necrotic environment. Necrosis often leads to the release of enzymes or other cell contents that may be used to trigger cleavage of a linker. In some embodiments, cleavage of the linker occurs by necrotic enzymes (e.g., by calpains).

For additional details regarding linkers and their use in the compounds described herein, see: Wu, A. M.; Senter, P. D. Arming antibodies: prospects and challenges for immunoconjugates. Nat. Biotechnol. 2005, 23(9): 1137-1146; Beck, A.; et al. The next generation of antibody-drug conjugates comes of age. Discov. Med. 2010, 10(53): 329-339; Nolting, B.; et al. Linker technologies for antibody-drug conjugates. Methods. Mol. Biol. 2013, 1045: 71-100; Jain, N.; et al. Current ADC linker chemistry. Pharm. Res. 2015, 32: 3526-3540; McCombs, J. R.; Owen, S. C. Antibody drug conjugates: design and selection of linker, payload and conjugation chemistry. AAPS J. 2015, 17(2): 339-351; Jun Lu,et al., Linkers Having a Crucial Role in Antibody-Drug Conjugates, Int J Mol Sci. 2016 April; 17(4): 561; each of which is incoproated by reference for such linker disclosures.

In some embodiments, the not cleavable linker comprises one or more of unsubstituted or substituted alkylene, unsubstituted or substituted cycloalkylene, unsubstituted or substituted heterocycloalkylene, unsubstituted or substituted arylene, and unsubstituted or substituted heteroarylene.

In some embodiments, L is absent, a cleavable linker, or a non-cleavable linker. In some embodiments, L is absent or comprises one or more amino acids, PEG groups, -L¹-, -L¹-L²-, -L¹-L²-L³-, -L¹-L²-L³-L⁴-, -L¹-L²-L³-L⁴-L⁵-, -L²-, -L²-L³-, -L²-L³-L⁴-, -L²-L³-L⁴-L⁵- , -L³-, -L³-L⁴-, -L³-L⁴-L⁵-, -L⁴-, -L⁴-L⁵-, -L⁵-, -L¹-L²-L³-L⁴-L⁵-, or a combination thereof.

In some embodiments, L is absent or comprises one or more amino acids, PEG groups, -L¹-, -L²-, -L³-, -L⁴-, -L⁵-, -L¹-L²-L³-L⁴-L⁵-, or a combination thereof.

In some embodiments, each L¹ is independently absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, one or more amino acids, —(CH₂)ₚ—, —C(═O)—, —(CH₂)ₚ—C(═O)—, —C(═O)—(CH₂)ₚ—, —(CH₂)ₚ—C(═O)—(CH₂)ₚ—, —C(═O)NH—, —(CH₂)ₚ—C(═O)NH—, —C(═O)NH—(CH₂)ₚ—, —(CH₂)ₚ—C(═O)NH—(CH₂)ₚ—, each p is independently 0, 1, 2, 3, 4, 5, 6, 7, 8, 9, 10, 11, or 12. In some embodiments, each L¹ is independently absent, unsubstituted or substituted alkylene, unsubstituted or substituted heteroalkylene, unsubstituted or substituted monocyclic cycloalkylene, unsubstituted or substituted monocyclic heterocycloalkylene, unsubstituted or substituted phenylene, unsubstituted or substituted monocyclic heteroarylene, one or more amino acids, —(CH₂)_{q}—, —C(═O)—, —(CH₂)ₚ—C(═O)—, —C(═O)—(CH₂)ₚ—, —(CH₂)ₚ—C(═O)—(CH₂)ₚ—, —C(═O)NH—, —(CH₂)ₚ—C(═O)NH—, —C(═O)NH—(CH₂)ₚ—, —(CH₂)ₚ—C(═O)NH—(CH₂)ₚ—, each p is independently 1, 2, 3, 4, 5, 6, 7, 8, 9, 10, 11, or 12.

In some embodiments, each L² is independently absent, —O—, —S—, —S(═O)—, —S(═O)₂—, —NH—, —CH(OH)—, —NHC(═O)—, —C(═O)O—, —OC(═O)—, —CH(═N)—, —CH(═N—NH)—, —CCH₃(═N)—, —CCH₃(═N—NH)—, —OC(═O)NH—, —NHC(═O)NH—, —NHC(═O)O—, —(CH₂)ₚ—, —C(═O)—(CH₂CH₂X)ₚ—, or —(CH₂CH₂X)ₚ—, p is 1, 2, 3, 4, 5, 6, 7, 8, 9, 10, 11, or 12; each X is independently selected from O, S, and NR^{X}; and R^{X} is hydrogen or C₁-C₄alkyl. In some embodiments, each L² is independently —C(═O)—, —C(═O)NH—, —C(═O)O—, —(CH₂)ₚ—, —C(═O)—(CH₂CH₂O)ₚ—, or —(CH₂CH₂O)ₚ—, each p is independently 1, 2, 3, 4, 5, 6, 7, 8, 9, 10, 11, or 12.

In some embodiments, each L³ is independently absent, unsubstituted or substituted alkylene, unsubstituted or substituted heteroalkylene, unsubstituted or substituted alkenylene, unsubstituted or substituted alkynylene, unsubstituted or substituted cycloalkylene, unsubstituted or substituted heterocycloalkylene, unsubstituted or substituted arylene, unsubstituted or substituted heteroarylene, one or more amino acids, —(CH₂)_{q}—, —(CH₂CH₂X)_{q}—, or —(XCH₂CH₂)_{q}—, each q is independently 1, 2, 3, 4, 5, 6, 7, 8, 9, 10, 11, or 12; each X is independently selected from O, S, and NR^{X}; and R^{X} is hydrogen or C₁-C₄alkyl. In some embodiments, each L³ is independently unsubstituted or substituted alkylene, unsubstituted or substituted heteroalkylene, —(CH₂)_{q}—, each q is independently 1, 2, 3, 4, 5, 6, 7, 8, 9, 10, 11, or 12.

In some embodiments, each L⁴ is independently absent, —O—, —S—, —S(O)—, —S(O)₂—, —NH—, —CH(OH)—, —C(═O)—, —C(═O)NH—, —NHC(═O)—, —C(═O)O—, —OC(═O)—, —OC(═O)NH—, —NHC(═O)NH—, or —NHC(═O)O—. In some embodiments, each L⁴ is independently absent, —NH—.

In some embodiments, each L⁵ is independently absent, unsubstituted or substituted alkylene, or unsubstituted or substituted heteroalkylene. In some embodiments, each L⁵ is independently absent or unsubstituted or substituted alkylene.

In some embodiments, L is absent or a linker that is -L¹-, -L²-, -L³-, -L⁴-, -L⁵-, -L¹-L²-L³-L4-L⁵-, or a combination thereof. In some embodiments, L is absent or a linker that is -L¹-, -L²-, -L³-, -L⁴-, -L⁵-, -L¹-L²-L³-L⁴-L⁵-, or a combination thereof; each L¹ is independently absent, unsubstituted or substituted alkylene, unsubstituted or substituted heteroalkylene, unsubstituted or substituted alkenylene, unsubstituted or substituted alkynylene, unsubstituted or substituted monocyclic cycloalkylene, unsubstituted or substituted monocyclic heterocycloalkylene, unsubstituted or substituted phenylene, unsubstituted or substituted monocyclic heteroarylene, one or more amino acids, —(CH₂)ₚ—, —(CH₂)ₚ—,—C(═O)—, —C(═O)—(CH₂)ₚ—, —C(═O)NH—,—C(═O)NH—(CH₂)ₚ—, each p is independently 1, 2, 3, 4, 5, 6, 7, 8, 9, 10, 11, or 12; each L² is independently —C(═O)—, —C(═O)NH—, —C(═O)O—, —(CH₂)ₚ—, —C(═O)—(CH₂CH₂O)ₚ—, or —(CH₂CH₂O)ₚ—, each p is independently 1, 2, 3, 4, 5, 6, 7, 8, 9, 10, 11, or 12; each L³ is independently unsubstituted or substituted alkylene, unsubstituted or substituted heteroalkylene, —(CH₂)_{q}—, each q is independently 1, 2, 3, 4, 5, 6, 7, 8, 9, 10, 11, or 12; each L⁴ is independently absent, —NH—; each L⁵ is independently absent, unsubstituted or substituted alkylene, or unsubstituted or substituted heteroalkylene.

In some embodiments, -L²-L³-L⁴-L⁵− is

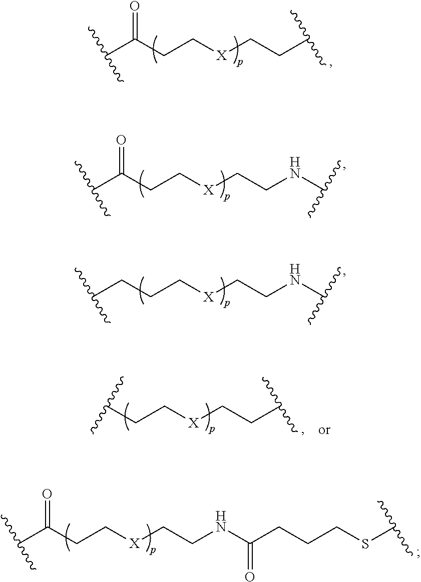

each p is independently 1, 2, 3, 4, 5, 6, 7, 8, 9, 10, 11, or 12; and each X is independently O or NR^{X}; and R^{X} is hydrogen or C₁-C₄alkyl.

In some embodiments, -L²-L³-L⁴-L⁵- is

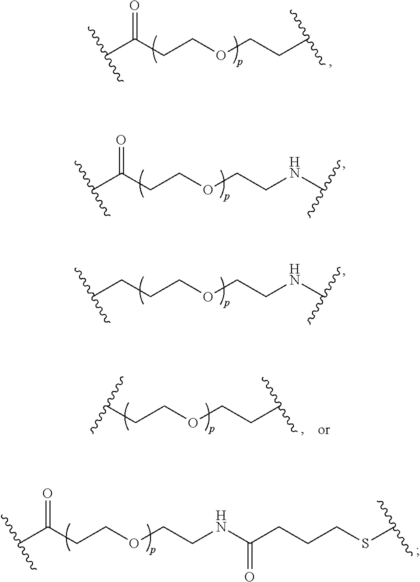

each p is independently 1, 2, 3, 4, 5, 6, 7, 8, 9, 10, 11, or 12.

In some embodiments, -L²-L³-L⁴-L⁵- is

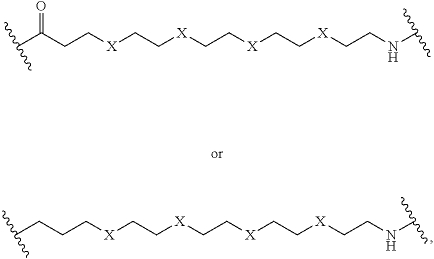

wherein each X is independently O or NR^{X}; and R^{X} is hydrogen or C₁-C₄alkyl.

In some embodiments, L is

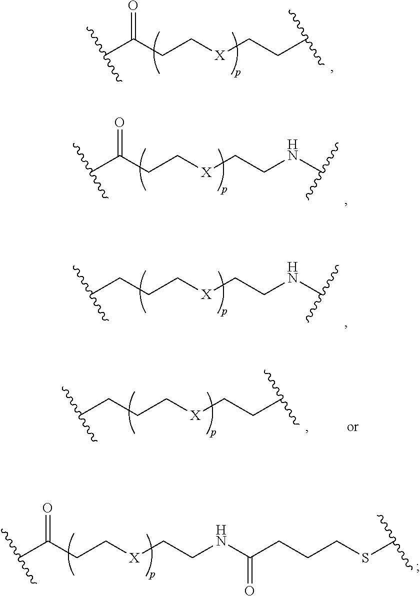

each p is independently 1, 2, 3, 4, 5, 6, 7, 8, 9, 10, 11, or 12; and each X is independently O or NR^{X}; and R^{X} is hydrogen or C₁-C₄alkyl.

In some embodiments, L is

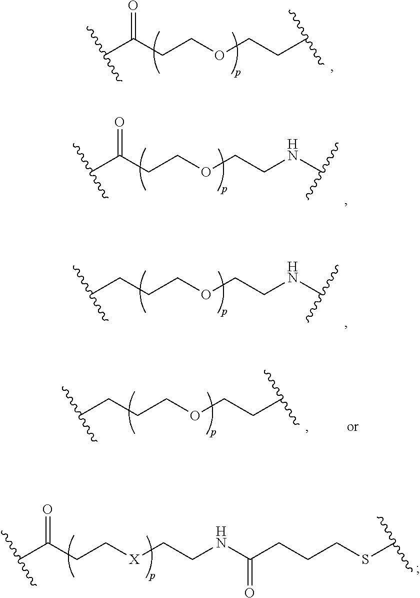

each p is independently 1, 2, 3, 4, 5, 6, 7, 8, 9, 10, 11, or 12.

In some embodiments, L is

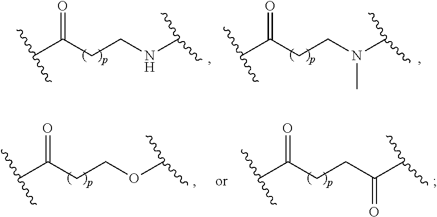

each p is independently 1, 2, 3, 4, 5, 6, 7, 8, 9, 10, 11, or 12.

In some embodiments, L is:

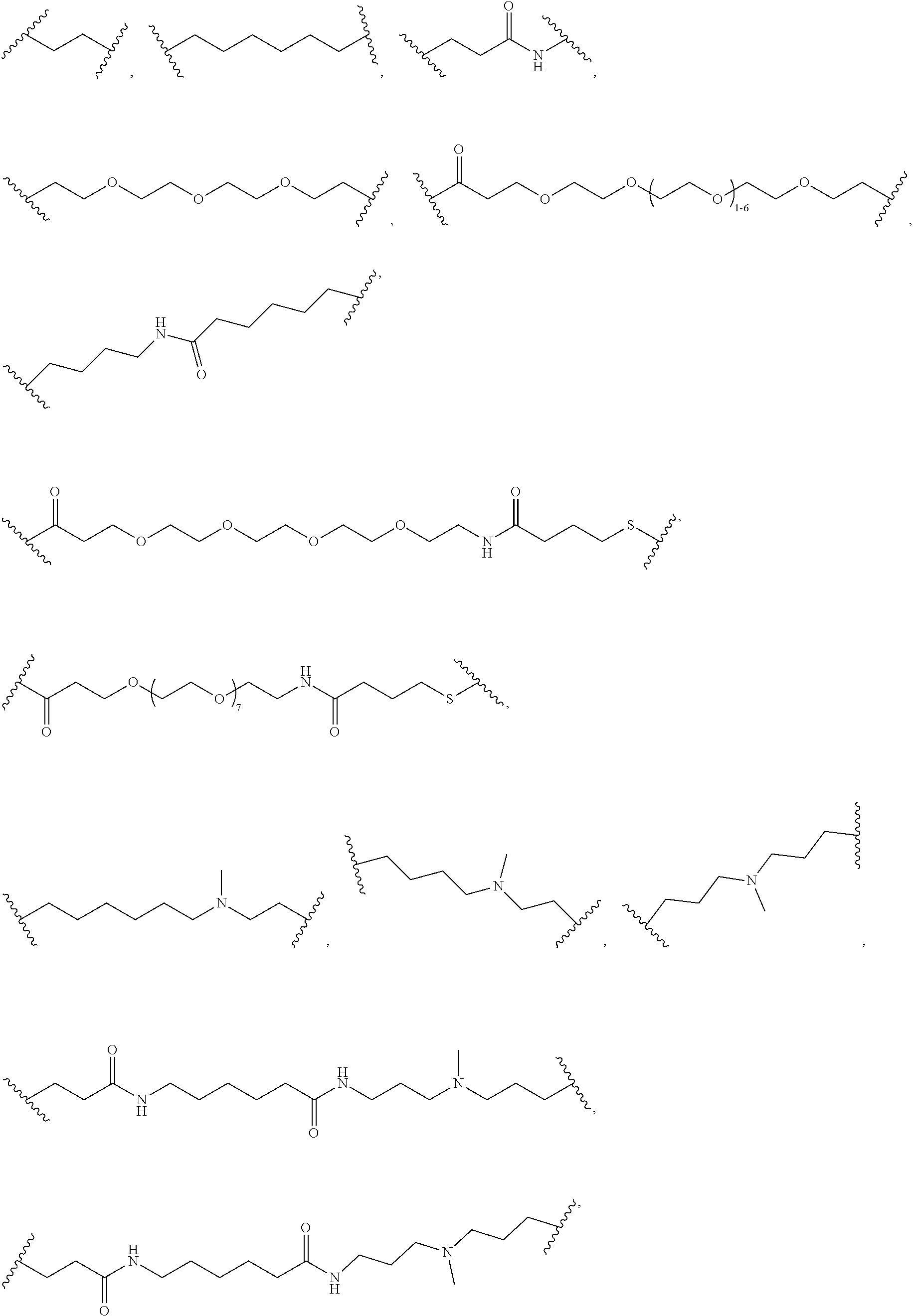

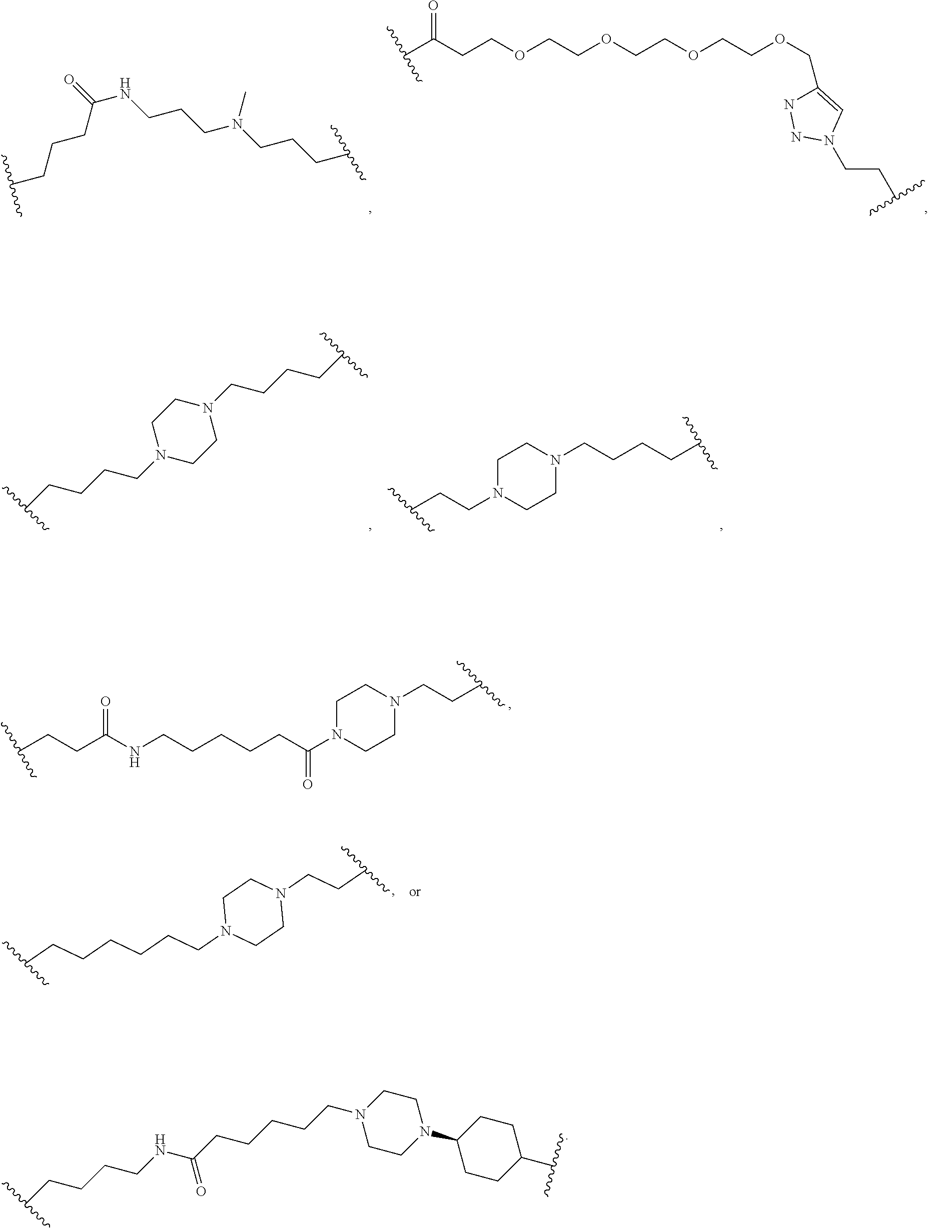

In some embodiments, the linker comprises a click chemistry residue. In some embodiments, the linker is attached to a non-peptide ligand, to a metal chelator or both via click chemistry. For example, in some embodiments, a non-peptide ligand comprises an azide group that reacts with an alkyne moiety of the linker. For another example, in some embodiments, a non-peptide ligand comprises an alkyne group that reacts with an azide of the linker. The metal chelator and the linker can be attached similarly. In some embodiments, the linker comprises an azide moiety, an alkyne moiety, or both. In some embodiments, the linker comprises a triazole moiety.

### Representative Linker and Payload Moieties

In some embodiments, -L-Q is:

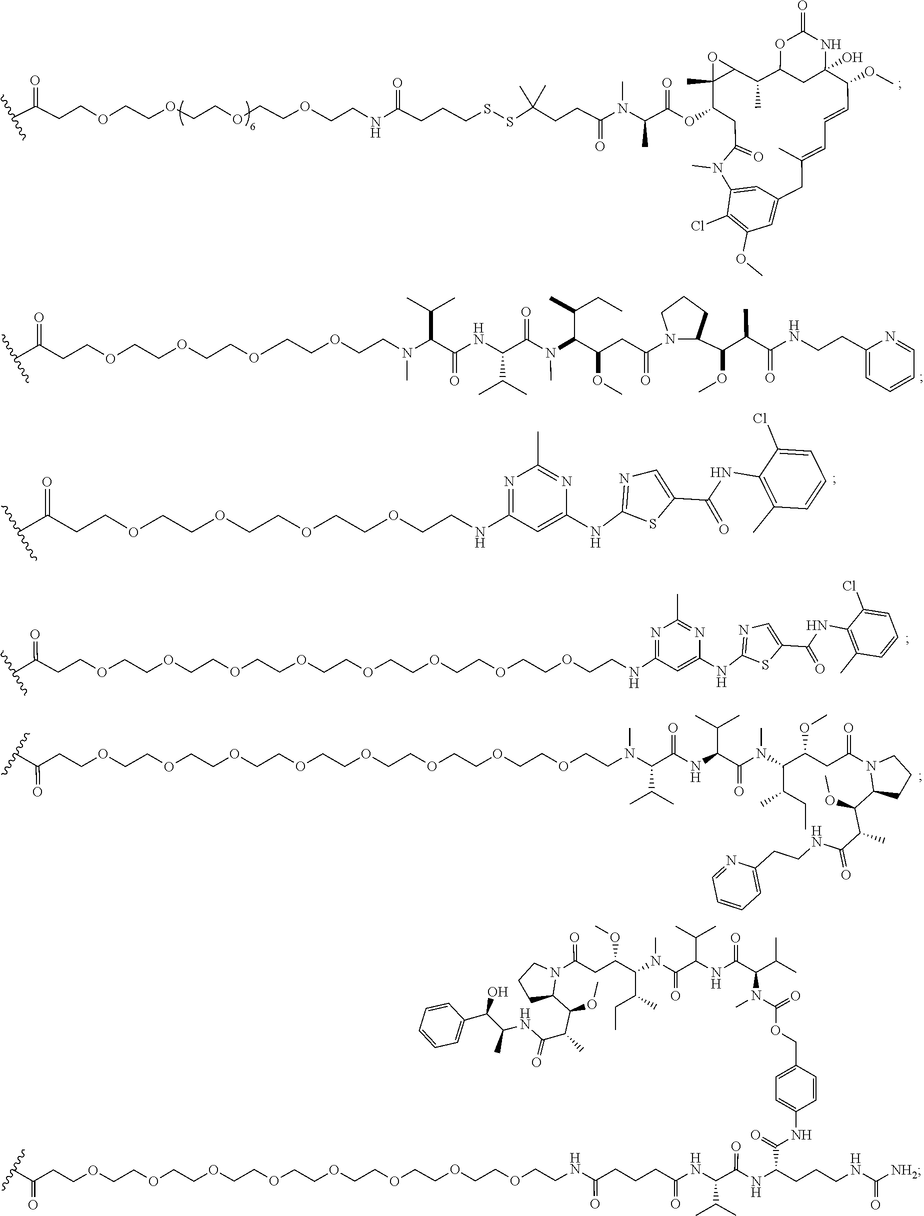

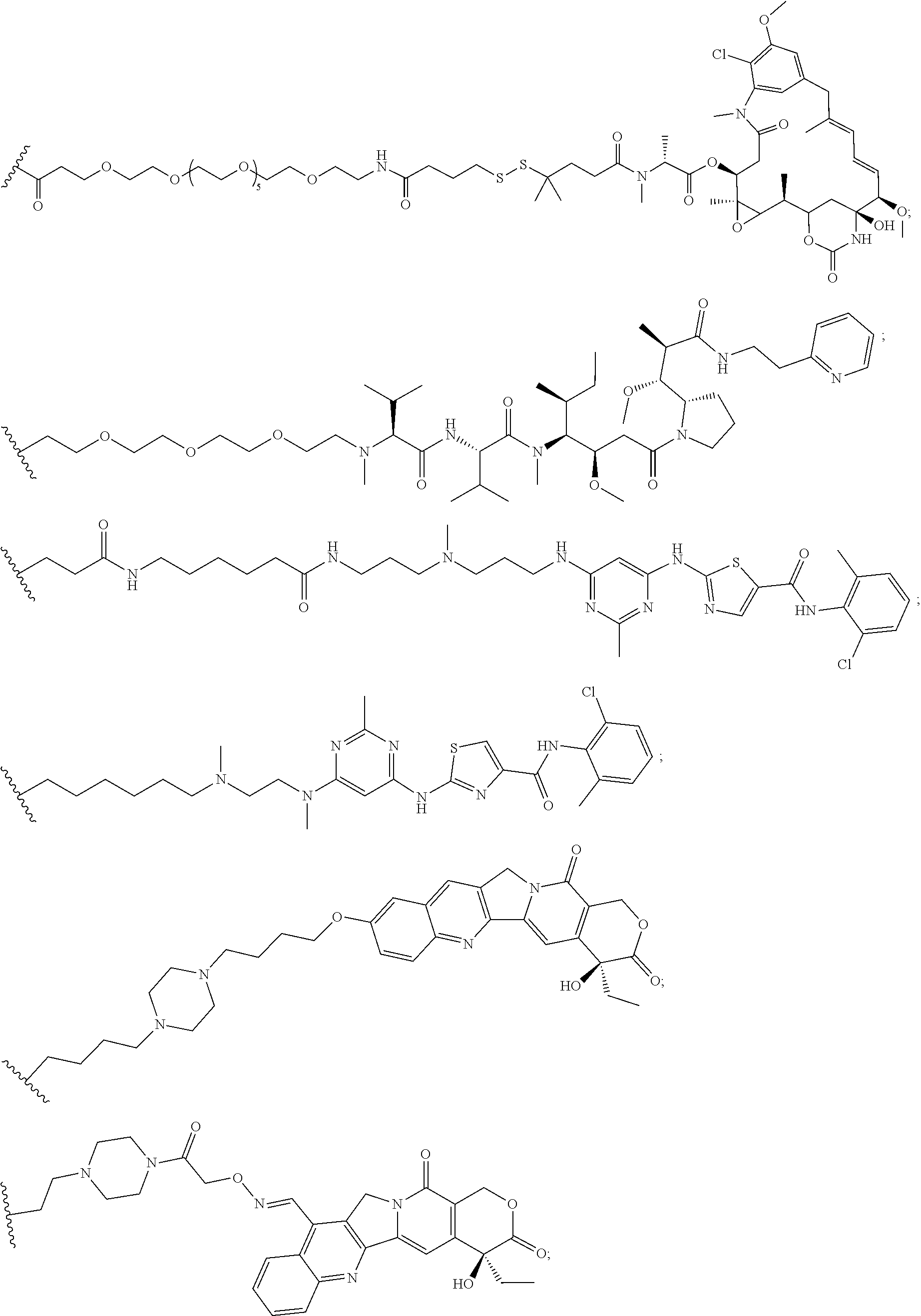

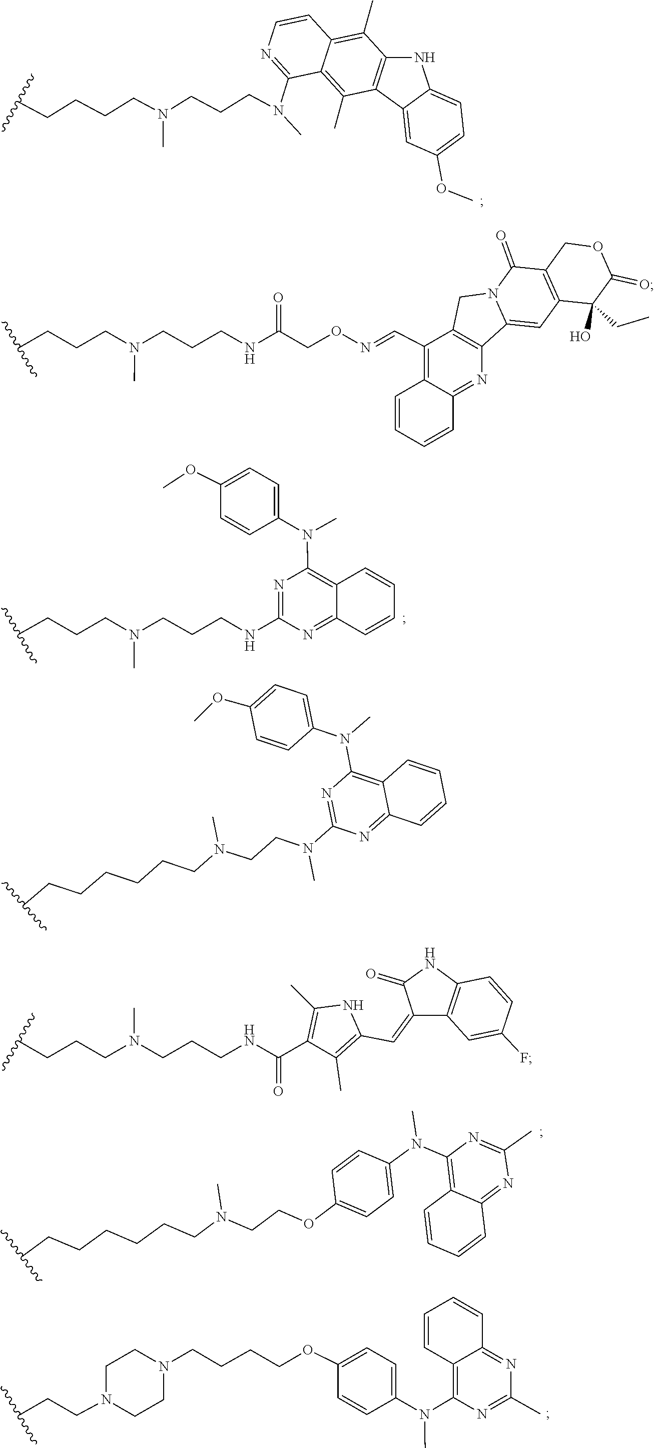

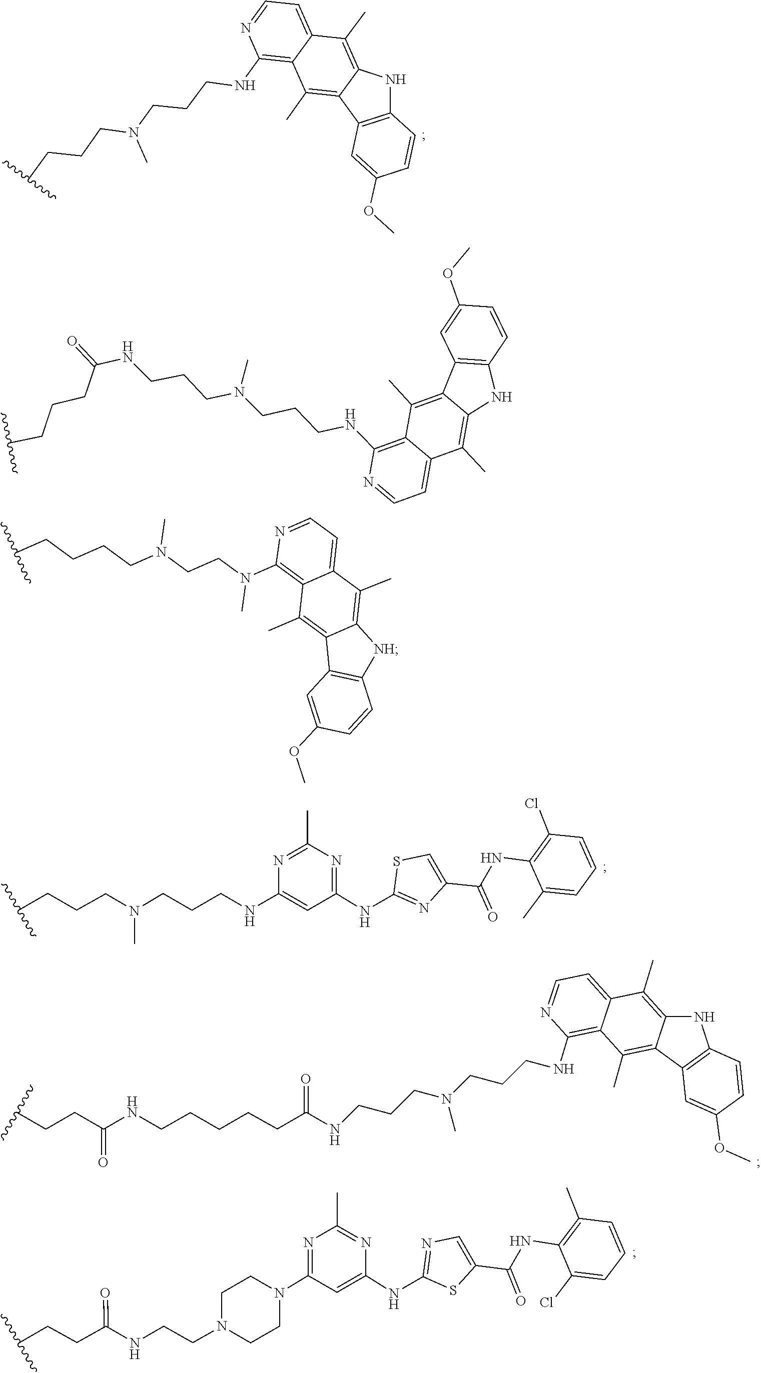

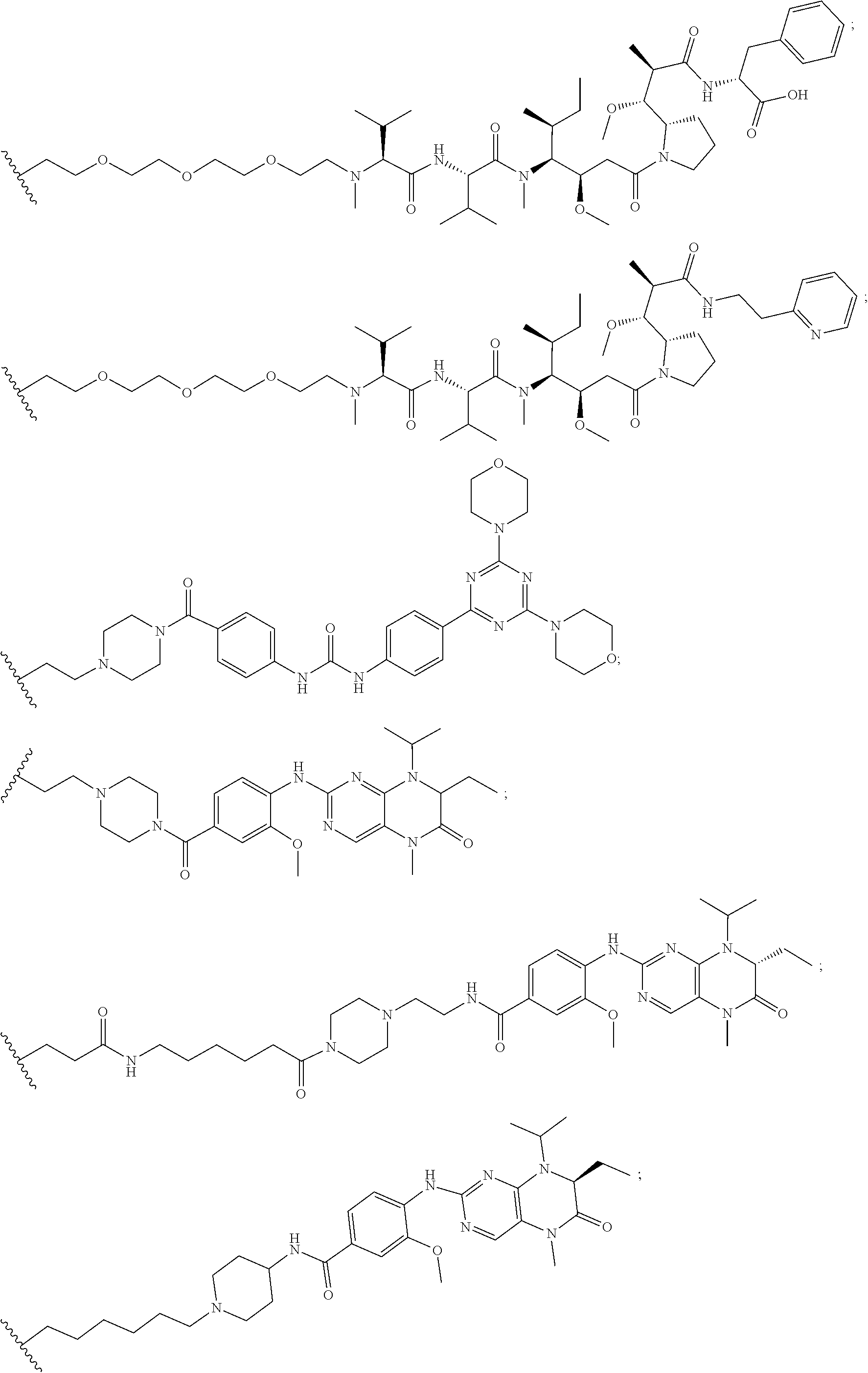

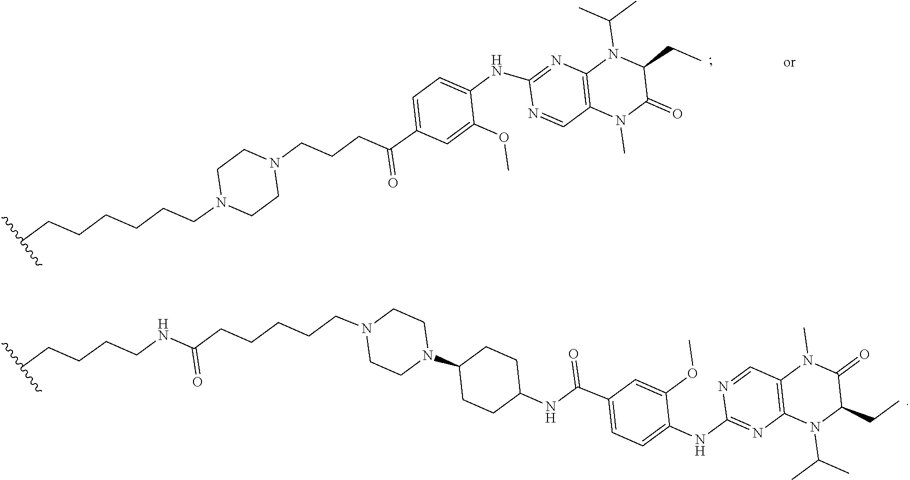

### Representative Non-Peptide Small Molecule Drug Conjugates (NPDCs)

As used herein, “non-peptide ligand” means a compound that is a small molecule. As used herein, “non-peptide ligand” means a compound that is a small molecule with a molecular weight <900 Daltons. A non-peptide ligand is not derived from chains of amino acids linked by peptide bonds. A non-peptide ligand is not an oligopeptide (e.g. dipeptide, tripeptide, tetrapeptide). Larger structures such as nucleic acids, proteins, and polysaccharides are not small molecules.

In some embodiments, NP is a non-peptide ligand that binds to tumor cells expressing somatostatin receptors.

In some embodiments, NP is a non-peptide ligand for the somatostatin receptor, wherein NP is a compound described in U.S. Pat. No. 10,696,689, US Patent Publication Number US20200010453, each of which is herein incorporated by reference for such compounds. In some embodiments, the non-peptide ligand is a compound described in any one of Formulas (I), (Ia), (Ib), (Ic), (Id), (II), (IIa), (IIb), (IIc), (IId), (III), (IIIa), (IIIb), (IIIc), or (IIId), of U.S. Pat. No. 10,696,689. In some embodiments, the non-peptide ligand is a compound described in Table 1, Table 2, or Table 3 of U.S. Pat. No. 10,696,689. In some embodiments, the non-peptide ligand is a compound described in Formula (I), (II), (III), (IV), (V), (VI), (VII), (VIII), (IX), (X), or (XI) of US20200010453. In some embodiments, the non-peptide ligand is a compound described in Table 1, Table 2, Table 3, or Table 4 of International Patent Application Publication Number WO 2018/170284.

In some embodiments, the non-peptide ligand is a compound described in U.S. Pat. Nos. 9,643,951, 9,630,976, US20200000816, each of which is herein incorporated for such compounds.

In some embodiments, NP is a non-peptide ligand comprising a 4-(4-aminopiperidin-1-yl)-5-(phenyl)pyridine structural motif or a 4-[(4αS,8αS)-octahydro-1H-pyrido[3,4-b][1,4]oxazin-6-yl]-5-(phenyl)pyridine structural motif. In some embodiments, NP is a non-peptide ligand comprising a 4-(4-aminopiperidin-1-yl)-5-(phenyl)pyridine structural motif or a 4-[(4αS,8αS)-octahydro-1H-pyrido[3,4-b][1,4]oxazin-6-yl]-5-(phenyl)pyridine structural motif, wherein -L-Q is attached to NP at the 2-position of the pyridine.

In some embodiments, NP has a structure of Formula (II), or a pharmaceutically acceptable salt or pharmaceutically acceptable solvate thereof:

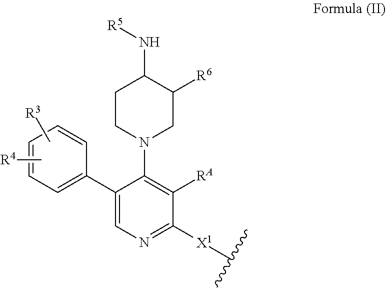

wherein:
R^{A} is

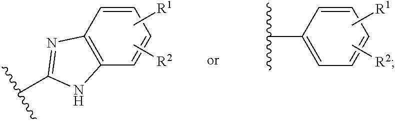

each R¹, R², R³ and R⁴ is independently hydrogen, halogen, substituted or unsubstituted C₁-C₄alkyl, substituted or unsubstituted C₁-C₄fluoroalkyl, substituted or unsubstituted C₁-C₄heteroalkyl, —CN, —N(R⁷)₂, or —OR⁷;
R⁵ is hydrogen, or substituted or unsubstituted C₁-C₆alkyl;
R⁶ is hydrogen, —OR⁷, —N(R⁷)₂, —CN, halogen, C₁-C₆ alkyl, or C₁-C₆fluoroalkyl;
or R⁵ and R⁶ are taken together with the intervening atoms to which they are attached to form a morpholine;
X¹ is absent, —O—, —S—, —N(R⁷)—, —C(═O)—, —C(═O)N(R⁷)—, —C(═O)O—, —N(R⁷)C(═O)—, or a heterocycle;
   each R⁷ is independently hydrogen or substituted or unsubstituted C₁-C₆alkyl.

In some embodiments, NP has a structure of Formula (III), or a pharmaceutically acceptable salt or pharmaceutically acceptable solvate thereof:

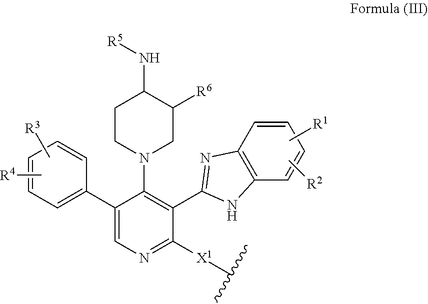

wherein:
each R¹, R², R³ and R⁴ is independently hydrogen, halogen, substituted or unsubstituted C₁-C₄alkyl, substituted or unsubstituted C₁-C₄fluoroalkyl, substituted or unsubstituted C₁-C₄heteroalkyl, —CN, —N(R⁷)₂, or —OR⁷;
R⁵ is hydrogen, or substituted or unsubstituted C₁-C₆alkyl;
R⁶ is hydrogen, —OR⁷, —N(R⁷)₂, —CN, halogen, C₁-C₆ alkyl, or C₁-C₆fluoroalkyl;
or R⁵ and R⁶ are taken together with the intervening atoms to which they are attached to form a morpholine;
X¹ is absent, —O—, —S—, —N(R⁷)—, —C(═O)—, —C(═O)N(R⁷)—, —C(═O)O—, —N(R⁷)C(═O)—, or a heterocycle; and
   each R⁷ is independently hydrogen or substituted or unsubstituted C₁-C₆alkyl.

In some embodiments, X¹ is absent, —O—, —S—, —N(R⁷)—, —C(═O)—, —C(═O)N(R⁷)—, —C(═O)O—, —N(R⁷)C(═O)—, azetidine, pyrrolidine, piperidine, or piperazine.

In some embodiments, X¹ is absent, —O—, —S—,

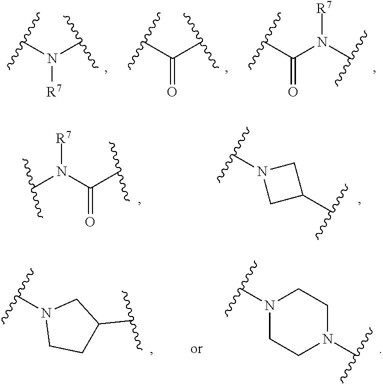

In some embodiments, NP has a structure of Formula (IV), or a pharmaceutically acceptable salt or pharmaceutically acceptable solvate thereof:

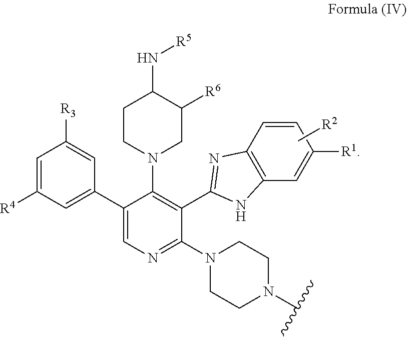

In some embodiments, each R¹, R², R³ and R⁴ is independently hydrogen, F, Cl, Br, —CN, —N(R⁷)₂, or C₁-C₄alkyl. In some embodiments, each R¹, R², R³ and R⁴ is independently hydrogen, F, C₁, —CH₃, —CH₂CH₃, or —OCH₃. In some embodiments, R⁵ is hydrogen; R⁶ is hydrogen, —OH, or —OCH₃; or R⁵ and R⁶ are taken together with the intervening atoms to which they are attached to form a morpholine. In some embodiments, each R⁷ is independently hydrogen or substituted or unsubstituted C₁-C₆alkyl. In some embodiments, each R⁷ is independently hydrogen or C₁-C₆alkyl. In some embodiments, each R⁷ is independently hydrogen, —CH₃, or —CH₂CH₃.

In some embodiments,

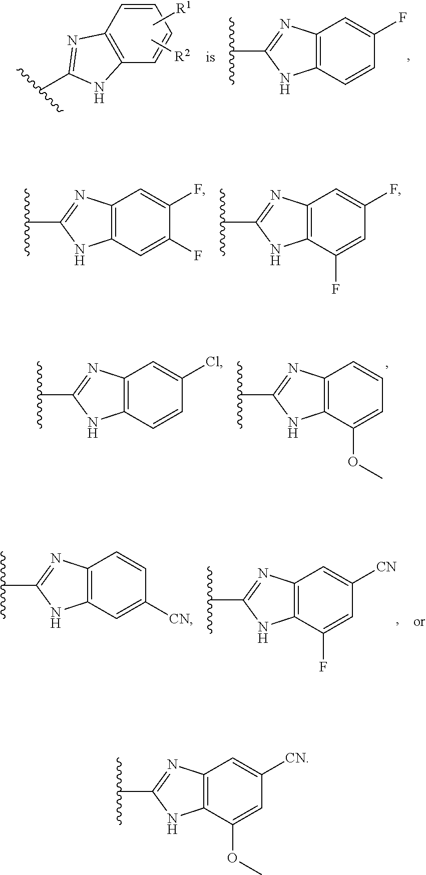

In some embodiments,

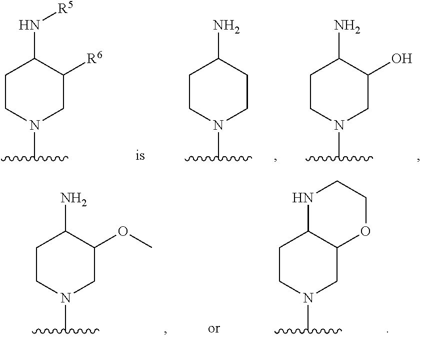

In some embodiments,

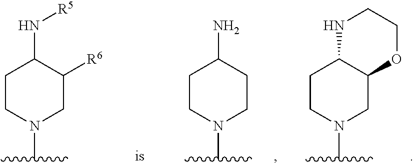

In some embodiments, R^{A} is

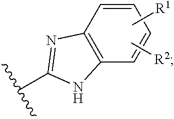

each R¹, R², R³ and R⁴ is independently hydrogen, F, Cl, Br, C₁-C₄alkyl, —CN, —N(R⁷)₂, or —OR⁷;
R⁵ is hydrogen; R⁶ is hydrogen or —OR⁷; or
R⁵ and R⁶ are taken together with the intervening atoms to which they are attached to form a morpholine; and
each R⁷ is independently hydrogen, —CH₃, or —CH₂CH₃.

In some embodiments,

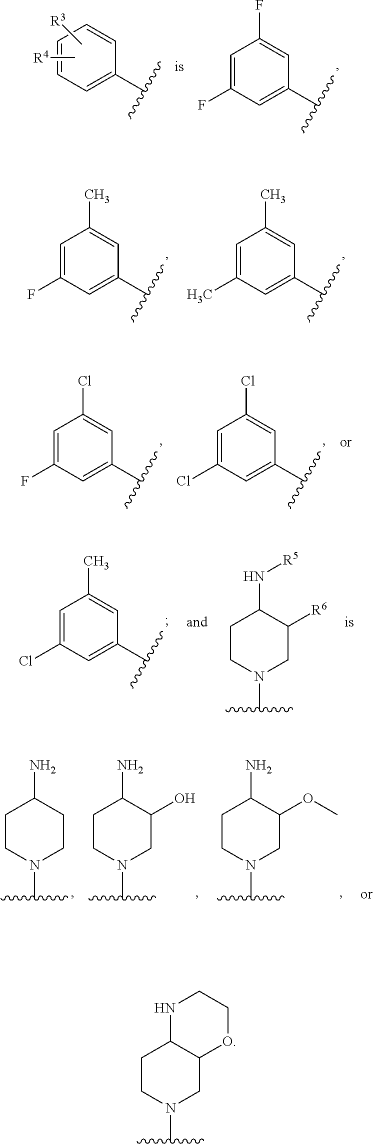

In some embodiments, is

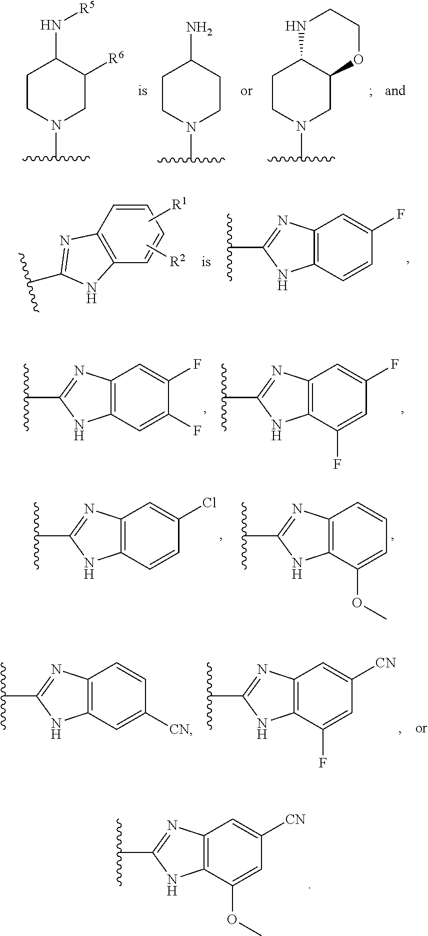

In some embodiments, the GPCR is the Somatostatin type 2 receptor (SSTR2); and NP has the following structure:

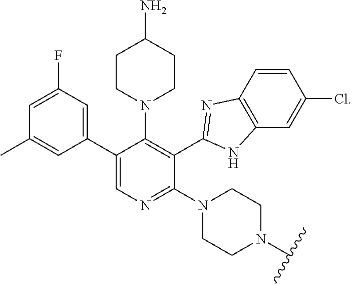 [SMILES: *N1CCN(c2ncc(-c3cc(C)cc(F)c3)c(N3CCC(N)CC3)c2-c2nc3ccc(Cl)cc3[nH]2)CC1]

In some embodiments, the compound has one of the following structures:

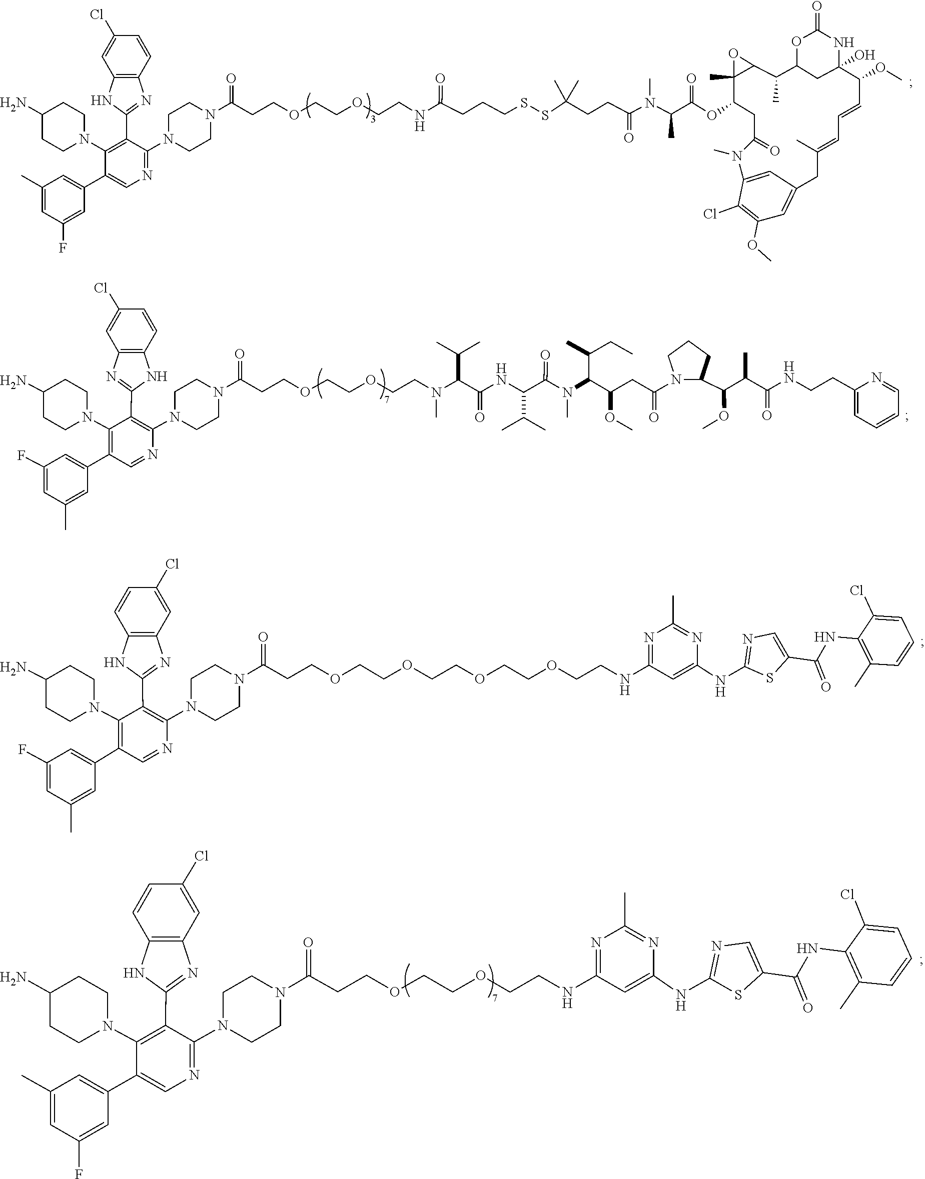

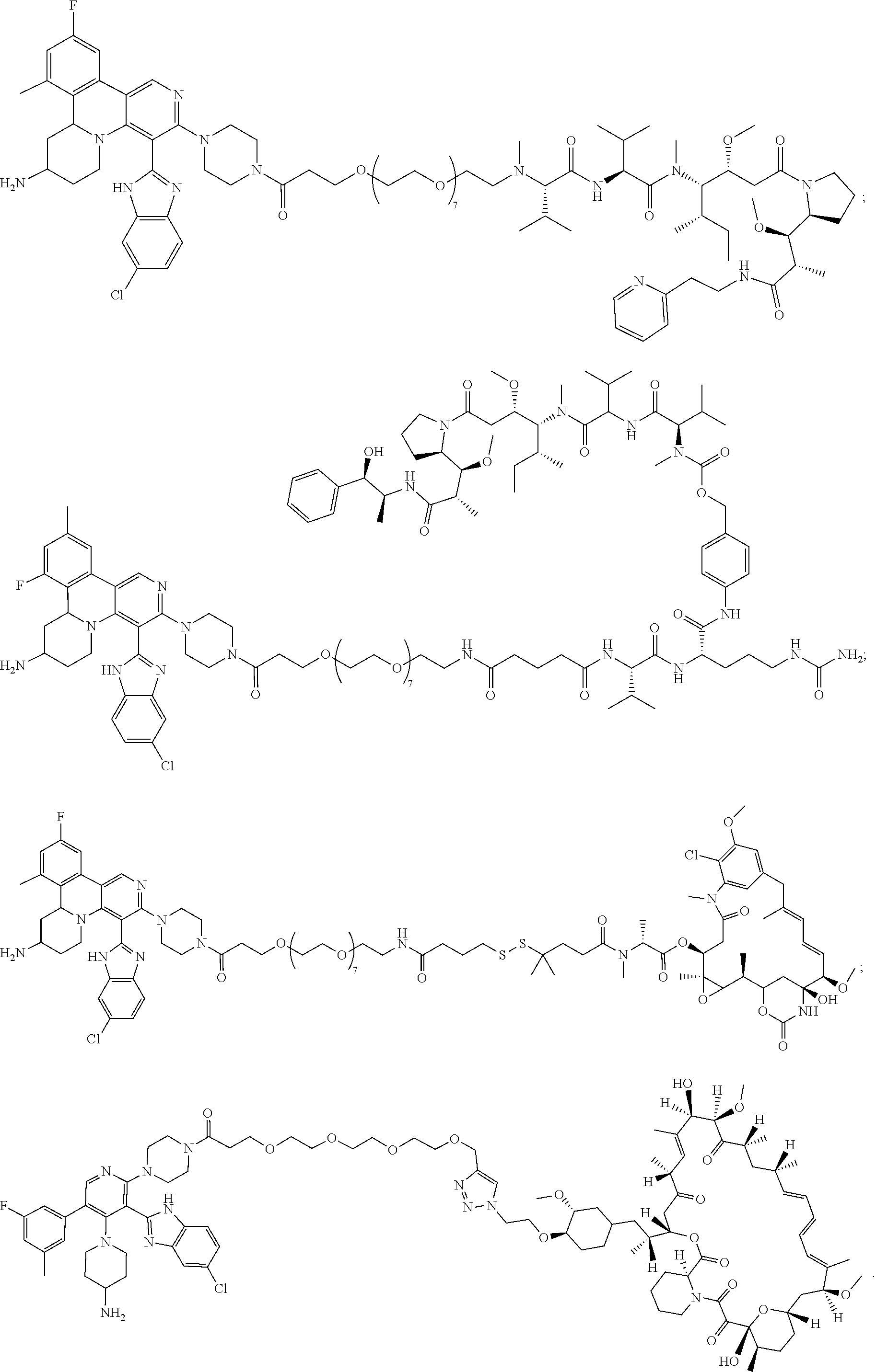

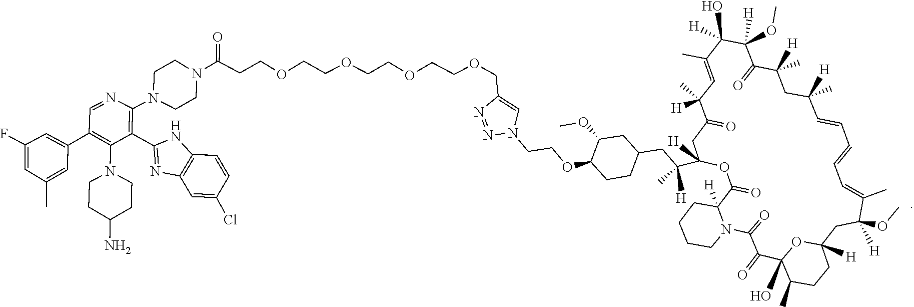 [SMILES: CO[C@H]1C[C@@H]2CC[C@@H](C)[C@@](O)(O2)C(=O)C(=O)N2CCCC[C@H]2C(=O)O[C@H]([C@H](C)CC2CC[C@@H](OCCn3cc(COCCOCCOCCOCCC(=O)N4CCN(c5ncc(-c6cc(C)cc(F)c6)c(N6CCC(N)CC6)c5-c5nc6cc(Cl)ccc6[nH]5)CC4)nn3)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, NP is a non-peptide ligand that binds to tumor cells expressing the gonadotropin-releasing hormone receptor (GnRHR).

In some embodiments, NP is a non-peptide ligand comprising a N-{4,6-dimethoxy-2-aminopyrimidin-5-yl}-5-[3,3,6-trimethyl-2,3-dihydro-1H-inden-5-yl)oxy]-2-furamide structural motif, a N-(4,6-dimethoxypyrimidin-5-yl)-5-(3,3,6-trimethyl-2,3-dihydro-1H-inden-5-yl)oxy)-2-furamide structural motif, or a N-(4,6-dimethoxypyrimidin-5-yl)-5-((3,3,6-trimethyl-2,3-dihydro-1H-inden-5-yl)oxy)furan-2-carboxamide structural motif.

In some embodiments, the GPCR is GnRHR; and NP has a structure of Formula (X), or a pharmaceutically acceptable salt or pharmaceutically acceptable solvate thereof:

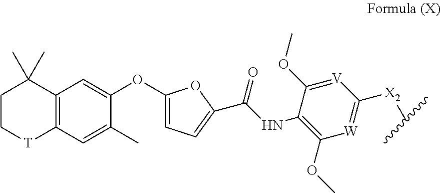

wherein:
V is CH or N; and W is CH or N;
T is absent, —CH₂—, —CH(CH₃)—, or —C(CH₃)₂—; and
X² is absent, —O—, or —N(R⁷)—; and
R⁷ is hydrogen or substituted or unsubstituted C₁-C₆alkyl.

In some embodiments, the GPCR is GnRHR; and NP has one of the following structures:

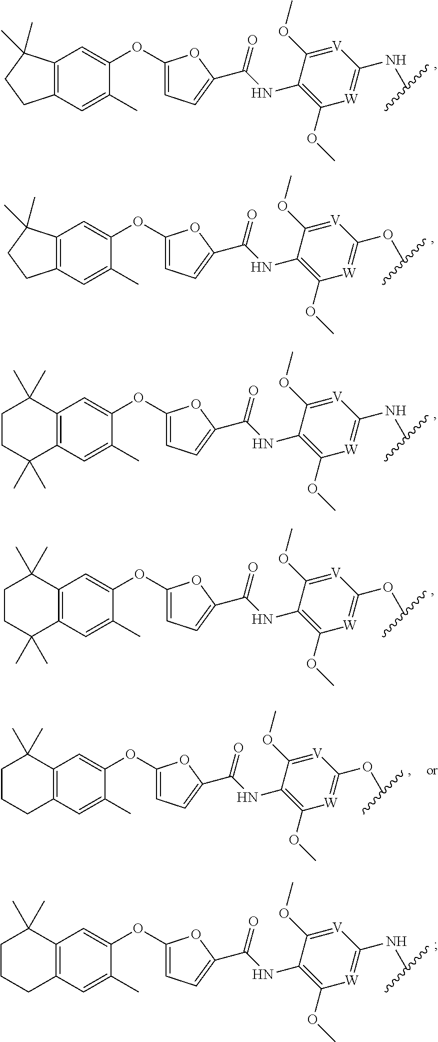

wherein, V is CH or N; and W is CH or N.

In some embodiments, the GPCR is GnRHR; and NP has one of the following structures:

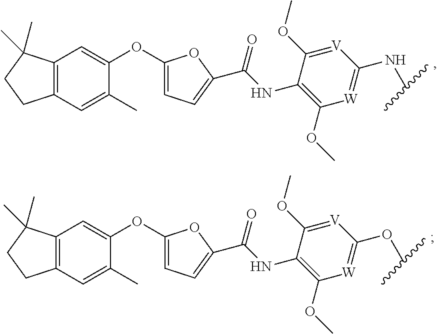

wherein, V is CH or N; and W is CH or N.

In some embodiments, the GPCR is GnRHR; and NP has one of the following wherein, V is CH or N; and W is CH or N.

In some embodiments, the GPCR is GnRHR; and NP has one of the following structures:

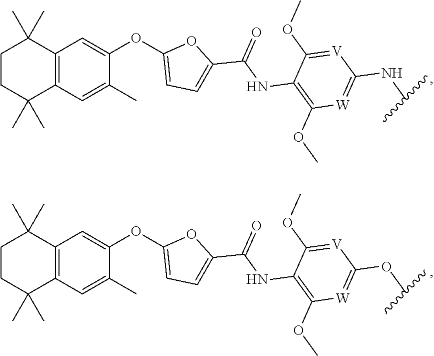

wherein, V is CH or N; and W is CH or N.

In some embodiments, the GPCR is GnRHR; and NP has one of the following structures:

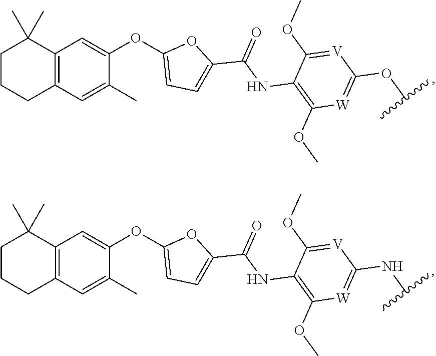

wherein, V is CH or N; and W is CH or N.

In some embodiments, the GPCR is GnRHR; and NP has the following structure:

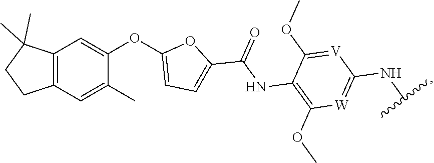

wherein, V is CH or N; and W is CH or N.

In some embodiments, the GPCR is GnRHR; and NP has the following structure:

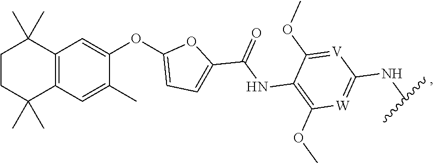

wherein, V is CH or N; and W is CH or N.

In some embodiments, the GPCR is GnRHR; and NP has the following structure:

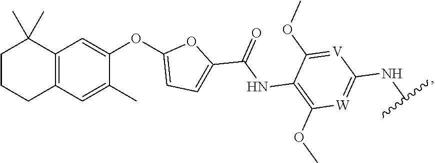

wherein, V is CH or N; and W is CH or N.

In some embodiments, the compound has one of the following structures:

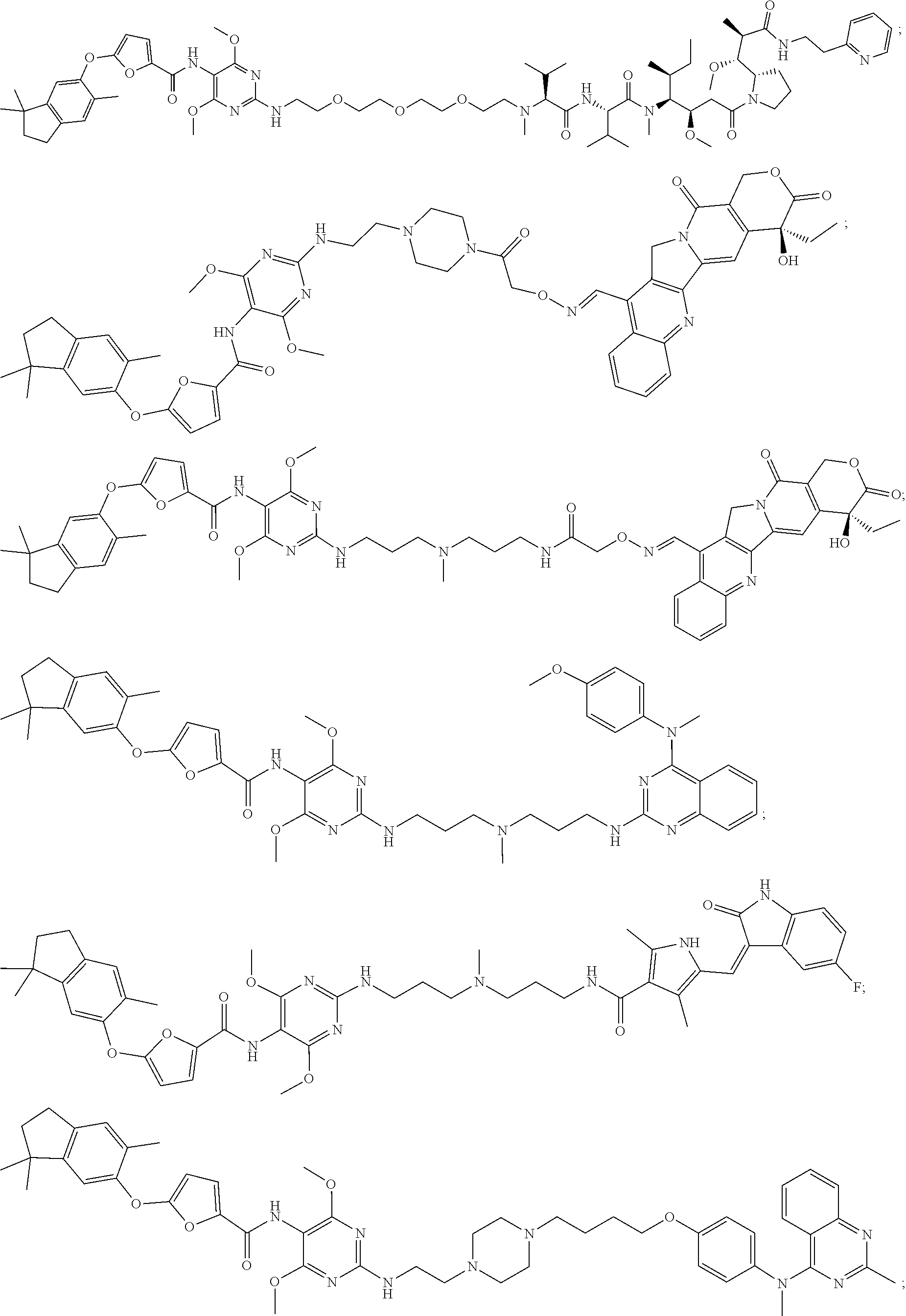

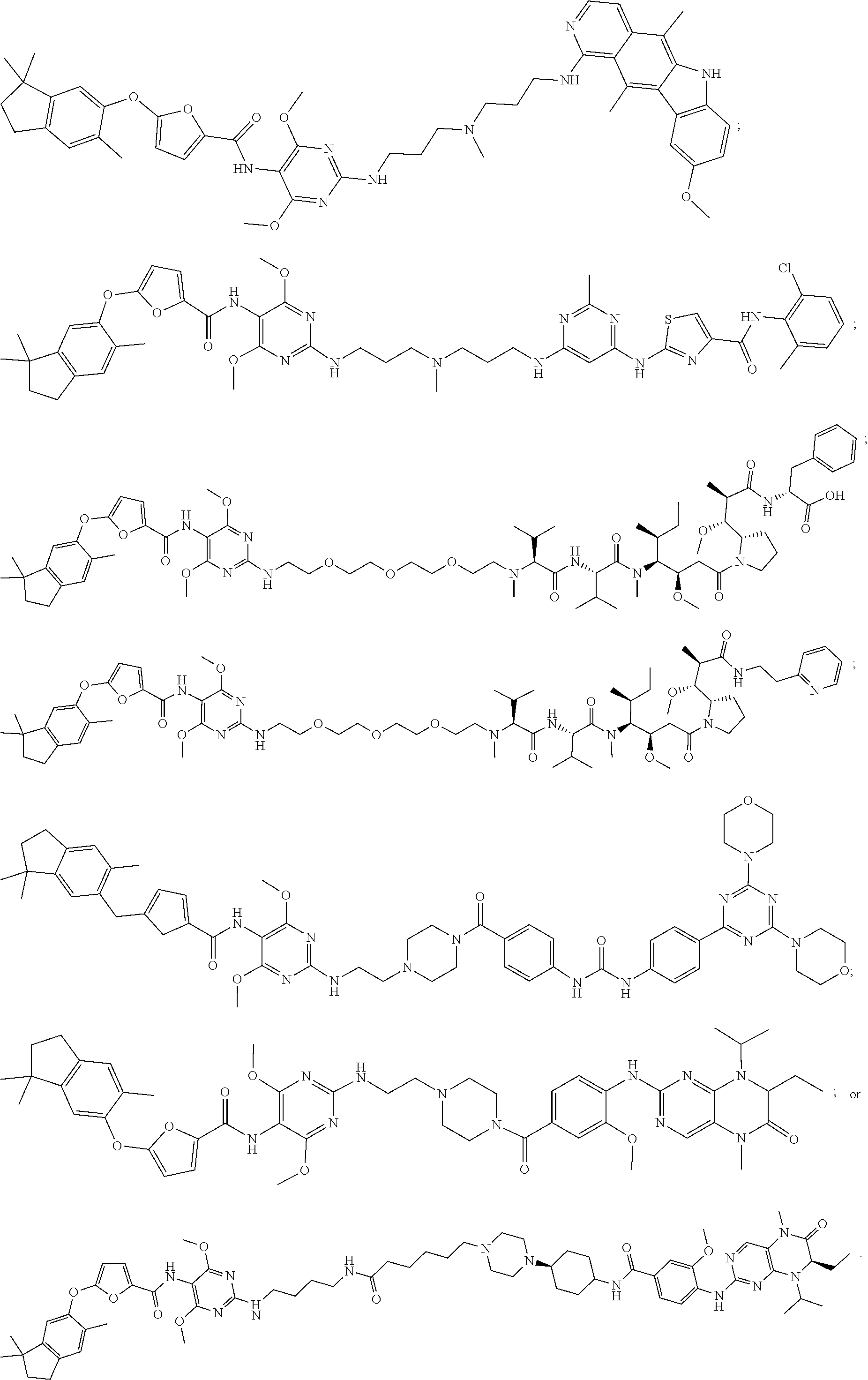

Any combination of the groups described above for the various variables is contemplated herein. Throughout the specification, groups and substituents thereof are chosen by one skilled in the field to provide stable moieties and compounds.

### Synthesis of Compounds

Compounds described herein are synthesized using standard synthetic techniques or using methods known in the art in combination with methods described herein.

Unless otherwise indicated, conventional methods of mass spectroscopy, NMR, HPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In one aspect, compounds described herein are in the form of pharmaceutically acceptable salts. In addition, the compounds described herein can exist in unsolvated as well as solvated forms with pharmaceutically acceptable solvents such as water, ethanol, and the like. The solvated forms of the compounds presented herein are also considered to be disclosed herein.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D.C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I) with an acid. In some embodiments, the compound of Formula (I) (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (-L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (-L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (I) is prepared as a chloride salt, sulfate salt, bromide salt, mesylate salt, maleate salt, citrate salt or phosphate salt.

In some embodiments, pharmaceutically acceptable salts are obtained by reacting a compound of Formula (I) with a base. In some embodiments, the compound of Formula (I) is acidic and is reacted with a base. In such situations, an acidic proton of the compound of Formula (I) is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, sites on the organic radicals (e.g. alkyl groups, aromatic rings) of compounds of Formula (I) are deuterated.

In some embodiments, the compounds of Formula (I) possess one or more stereocenters and each stereocenter exists independently in either the R or S configuration. In some embodiments, the compound of Formula (I) exists in the R configuration. In some embodiments, the compound of Formula (I) exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formula (I) are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference.

A “metabolite” of a compound disclosed herein is a derivative of that compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral), and parenteral routes (including injection or infusion, and subcutaneous).

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use.

### Methods of Treatment

In some embodiments, the methods comprise administering to a subject a therapeutically effective amount of a compound of Formula (I) or a pharmaceutically acceptable salt or solvate thereof. In some embodiments, the compound of Formula (I) or pharmaceutically acceptable salt or solvate thereof is administered in a pharmaceutical composition. In some embodiments, the subject has cancer. In some embodiments, the cancer is a solid tumor or hematological cancer. In some embodiments, the subject has a noncancerous tumor. In some embodiments, the subject has an adenoma.

In embodiments, the treatment is sufficient to reduce or inhibit the growth of the subject's tumor, reduce the number or size of metastatic lesions, reduce tumor load, reduce primary tumor load, reduce invasiveness, prolong survival time, or maintain or improve the quality of life, or combinations thereof.

In some embodiments, provided herein are methods for killing a tumor cell comprising contacting the tumor cell with a compound of Formula (I) or a pharmaceutically acceptable salt or solvate thereof.

In one aspect, provided herein are methods and compositions for treating cancers. Cancer includes tissue and organ carcinogenesis including metastases such as for example gastrointestinal cancer, (e.g., gastric cancer, esophageal cancer, pancreatic cancer colorectal cancer, intestinal cancer, anal cancer, liver cancer, gallbladder cancer, or colon cancer; lung cancer; thyroid cancer; skin cancer (e.g., melanoma); oral cancer; urinary tract cancer (e.g. bladder cancer or kidney cancer); blood cancer (e.g. myeloma or leukemia) or prostate cancer. In some embodiments, the present disclosure provides methods and compositions for treating gastrointestinal cancer in a subject in need thereof by administering an effective amount of a non-peptide targeted therapeutic compound disclosed herein to the subject. Non-limiting examples of gastrointestinal cancers that can be treated according to the methods of the present disclosure include gastric cancer, esophageal cancer, pancreatic cancer, lung cancer (small cell lung cancer and/or non small-cell lung cancer), colorectal cancer, intestinal cancer, anal cancer, liver cancer, gallbladder cancer, or colon cancer. In some embodiments, the cancer is Hodkin's lymphoma or B-cell lymphoma.

In one aspect, provided herein are methods and compositions for treating an adenoma.

In one aspect, provided herein are methods and compositions for treating a peptide hormone G protein-coupled receptor expressing cancer. In some embodiments, the peptide hormone G protein-coupled receptor-expressing cancer to be treated is a primary or metastatic cancer of gastrointestinal origin, such as colorectal cancer, stomach cancer, small intestine cancer, or esophageal cancer. In some embodiments, the peptide hormone G protein-coupled receptor-expressing cancer to be treated is primary or metastatic pancreatic cancer. In some embodiments, the peptide hormone G protein-coupled receptor-expressing cancer to be treated is primary or metastatic lung cancer, such as squamous cell carcinoma, adenosquamous carcinoma, or adenocarcinoma. In some embodiments, the peptide hormone G protein-coupled receptor-expressing cancer to be treated is a sarcoma, such as leiomyosarcoma or rhabdomyosarcoma. In some embodiments, the peptide hormone G protein-coupled receptor-expressing cancer to be treated is a primary or metastasized neuroectodermal tumor, such as aphaechromotcytoma or a paraganglioma. In some embodiments, the peptide hormone G protein-coupled receptor-expressing cancer is a primary or a metastasized bronchopulmonary or a gastrointestinal neuroendocrine tumor. In some embodiments, the cancer is colorectal cancer.

In another aspect, provided herein is a method for treating cancer in a mammal comprising administering to the mammal in need thereof, a non-peptide targeted therapeutic compound disclosed herein. In some embodiments, the cancer expresses one or more peptide hormone G protein-coupled receptors. In some embodiments, the cancer comprises a peptide hormone G protein-coupled receptor-positive cancer. In some embodiments, the cancer comprises a solid tumor. In some embodiments, the cancer comprises a sarcoma, carcinoma, or lymphoma. In some embodiments, the cancer comprises a neuroendocrine tumor. In some embodiments, the cancer comprises an insulinoma. In some embodiments, the cancer comprises peptide hormone G protein-coupled receptor-positive (e.g., somatostatin receptor-positive) gastroenteropancreatic neuroendocrine tumors (GEP-NETs).

### Methods of Dosing and Treatment Regimens

In one embodiment, compounds of Formula (I), or a pharmaceutically acceptable salt thereof, are used in the preparation of medicaments for the treatment of tumors in a mammal. Methods for treating any of the diseases or conditions described herein in a mammal in need of such treatment, involves administration of pharmaceutical compositions that include at least one compound of Formula (I) or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for diagnostic and/or therapeutic treatments.

The amount of a given agent that corresponds to such an amount varies depending upon factors such as the particular conjugate, specific cancer or tumor to be treated (and its severity), the identity (e.g., weight, sex) of the subject or host in need of treatment, but nevertheless is determined according to the particular circumstances surrounding the case, including, e.g., the specific conjugate being administered, the route of administration, the condition being treated, and the subject or host being treated. Optimal doses are generally determined using experimental models and/or clinical trials. The optimal dose depends upon the body mass, weight, or blood volume of the subject.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans.

The amount of conjugates or pharmaceutically acceptable salts or solvates thereof and/or pharmaceutical compositions administered can be sufficient to deliver a therapeutically effective dose of the particular subject. In some embodiments, conjugate dosages are between about 0.1 pg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of the conjugate or a pharmaceutically acceptable salt or solvate thereof described herein for methods of treating a disease as described herein is about 0.001 mg/kg to about 1 mg/kg body weight of the subject per dose. In some embodiments, the dose of conjugate or a pharmaceutically acceptable salt or solvate thereof described herein for the described methods is about 0.001 mg to about 1000 mg per dose for the subject being treated. In some embodiments, a conjugate or a pharmaceutically acceptable salt or solvate thereof described herein is administered to a subject at a dosage of from about 0.01 mg to about 500 mg, from about 0.01 mg to about 100 mg, or from about 0.01 mg to about 50 mg.

In some embodiments, a conjugate or a pharmaceutically acceptable salt or solvate thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole.

In some embodiments, the dose is administered once a day, 1 to 3 times a week, 1 to 4 times a month, or 1 to 12 times a year.

In any of the aforementioned aspects are further embodiments in which the effective amount of the compound of Formula (I), or a pharmaceutically acceptable salt thereof, is: (a) systemically administered to the mammal; and/or (b) administered orally to the mammal; and/or (c) intravenously administered to the mammal; and/or (d) administered by injection to the mammal.

### Combination Treatments

In certain instances, it is appropriate to administer at least one compound of Formula (I), or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In any case, regardless of the disease, disorder or condition being treated, the overall benefit experienced by the patient is simply be additive of the two therapeutic agents or the patient experiences a synergistic benefit.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a (alkyl)O— group, where alkyl is as defined herein.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl. In some embodiments, a cycloalkyl is a C₃-C₄cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, -SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion). Those of skill in the art are familiar with administration techniques that can be employed with the compounds and methods described herein.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (I), or a pharmaceutically acceptable salt thereof, and a co-agent, are both administered to a patient simultaneously in the form of a single entity or dosage. The term “non-fixed combination” means that the active ingredients, e.g. a compound of Formula (I), or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### EXAMPLES

The following examples are provided for illustrative purposes only and not to limit the scope of the claims provided herein.

### Synthesis of Compounds

### Example 1. N-[3-({3-[(4,6-dimethoxy-5-{5-[(3,3,6-trimethyl-2,3-dihydro-1H-inden-5-yl)oxy]furan-2-amido}pyrimidin-2-yl)amino]propyl}(methyl)amino)propyl]-5-{[(3Z)-5-fluoro-2-oxo-2,3-dihydro-1H-indol-3-ylidene]methyl}-2,4-dimethyl-1H-pyrrole-3-carboxamide (Compound 1)

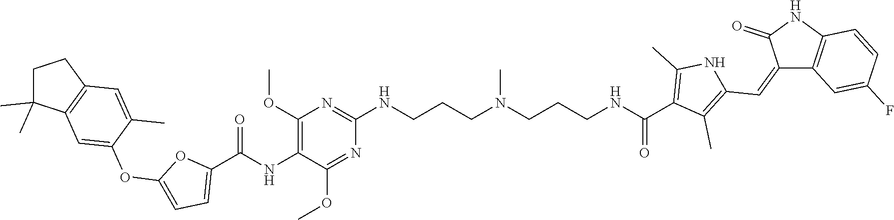 [SMILES: COc1nc(NCCCN(C)CCCNC(=O)c2c(C)[nH]c(/C=C3\C(=O)Nc4ccc(F)cc43)c2C)nc(OC)c1NC(=O)c1ccc(Oc2cc3c(cc2C)CCC3(C)C)o1]

Step 1-1, preparation of 5-{[(3Z)-5-fluoro-2-oxo-2,3-dihydro-1H-indol-3-ylidene]methyl}-2,4-dimethyl-1H-pyrrole-3-carboxylic acid: to an EtOH (3.0 mL) of 5-fluoroindolin-2-one (151 mg, 1.0 Eq, 1.0 mmol) was added 5-formyl-2,4-dimethyl-1H-pyrrole-3-carboxylic acid (167 mg, 1.0 Eq, 1.0 mmol) and pyrrolidine (0.50 mL, 6.0 Eq, 6.1 mmol). The resulting mixture was heated at 65° C. for 3 hours. The reaction crude was acidified and diluted with water. The resulting suspension was filtered, and the filtrate was washed with water and hexane. The remaining solid was collected and dried under vacuum to give crude 5-{[(3Z)-5-fluoro-2-oxo-2,3-dihydro-1H-indol-3-ylidene]methyl}-2,4-dimethyl-1H-pyrrole-3-carboxylic acid (213 mg, 71%). This material was used for next step without further purification.

Step 1-2, preparation of N-[2-({3-[(3-aminopropyl)(methyl)amino]propyl}amino)-4,6-dimethoxypyrimidin-5-yl]-5-[(3,3,6-trimethyl-2,3-dihydro-1H-inden-5-yl)oxy]furan-2-carboxamide: to a MeCN (1.0 mL) solution of N-(2-chloro-4,6-dimethoxypyrimidin-5-yl)-5-[(3,3,6-trimethyl-2,3-dihydro-1H-inden-5-yl)oxy]furan-2-carboxamide (229 mg, 1.0 Eq, 0.50 mml) was added N1-(3-aminopropyl)-N1-methylpropane-1,3-diamine (289 mg, 4.0 Eq, 2.0 mmol). The resulting mixture was heated at 115° C. for 3 hours. The reaction crude was concentrated, diluted with ethyl acetate, washed with water, dried, and concentrated to give crude N-[2-({3-[(3-aminopropyl)(methyl)amino]propyl}amino)-4,6-dimethoxypyrimidin-5-yl]-5-[(3,3,6-trimethyl-2,3-dihydro-1H-inden-5-yl)oxy]furan-2-carboxamide (272 mg, 98%) as a colorless oil. This material was used for next step without further purification.

Step 1-3, preparation of N-[3-({3-[(4,6-dimethoxy-5-{5-[(3,3,6-trimethyl-2,3-dihydro-1H-inden-5-yl)oxy]furan-2-amido}pyrimidin-2-yl)amino]propyl}(methyl)amino)propyl}-5-{[(3Z)-5-fluoro-2-oxo-2,3-dihydro-1H-indol-3-ylidene]methyl}-2,4-dimethyl-1H-pyrrole-3-carboxamide: to a DMF (1.0 mL) solution of 5-{[(3Z)-5-fluoro-2-oxo-2,3-dihydro-1H-indol-3-ylidene]methyl}-2,4-dimethyl-1H-pyrrole-3-carboxylic acid (30 mg, 1.0 Eq, 0.10 mmol) was added N,N-diisopropylethylamine (DIPEA) (35 μL, 2.0 Eq, 0.20 mmol), N,N,N′,N′-tetramethyl-O-(1H-benzotriazol-1-yl)uronium hexafluorophosphate (HBTU) (76 mg, 2.0 Eq, 0.20 mmol) and N-[2-({3-[(3-aminopropyl)(methyl)amino]propyl}amino)-4,6-dimethoxypyrimidin-5-yl]-5-[(3,3,6-trimethyl-2,3-dihydro-1H-inden-5-yl)oxy]furan-2-carboxamide (56 mg, 1.0 Eq, 0.10 mmol). The resulting mixture was stirred at ambient temperature for 2 hours. The reaction mixture was diluted with ethyl acetate, washed with water and bine, dried, and concentrated. The remaining residue was purified by prep-TLC eluting with DCM/MeOH/NH₄OH (20/1/0.5) to give N-[3-({3-[(4,6-dimethoxy-5-{5-[(3,3,6-trimethyl-2,3-dihydro-1H-inden-5-yl)oxy]furan-2-amido}pyrimidin-2-yl)amino]propyl}(methyl)amino)propyl]-5-{[(3Z)-5-fluoro-2-oxo-2,3-dihydro-1H-indol-3-ylidene]methyl}-2,4-dimethyl-1H-pyrrole-3-carboxamide (12 mg, 140%). MS (M+H)⁺=849.5.

The following conjugates were prepared similarly to Example 1 with appropriate substituting reagents and substrates at different steps and they may require additional functional group modifications via well-known chemistry with appropriate reagents.

| Compound | MS |
|---|---|
| 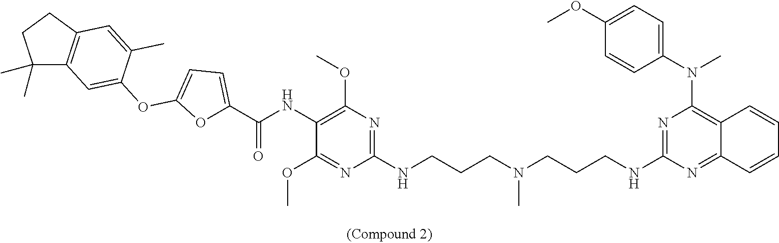 [SMILES: COc1ccc(N(C)c2nc(NCCCN(C)CCCNc3nc(OC)c(NC(=O)c4ccc(Oc5cc6c(cc5C)CCC6(C)C)o4)c(OC)n3)nc3ccccc23)cc1] | (M + H)⁺ = 830.5 |
| 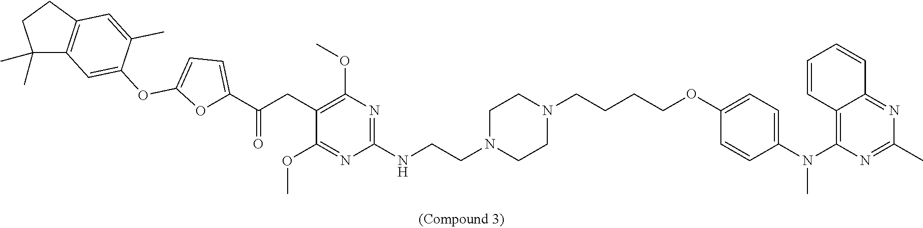 | (1/2M + H)⁺ = 435.8 |
| 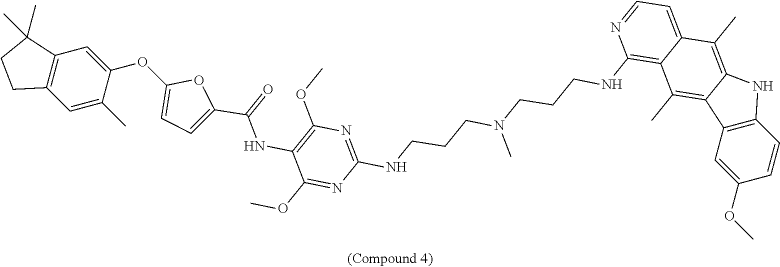 [SMILES: COc1ccc2[nH]c3c(C)c4ccnc(NCCCN(C)CCCNc5nc(OC)c(NC(=O)c6ccc(Oc7cc8c(cc7C)CCC8(C)C)o6)c(OC)n5)c4c(C)c3c2c1] | (1/2M + H)⁺ = 421.4 |
| 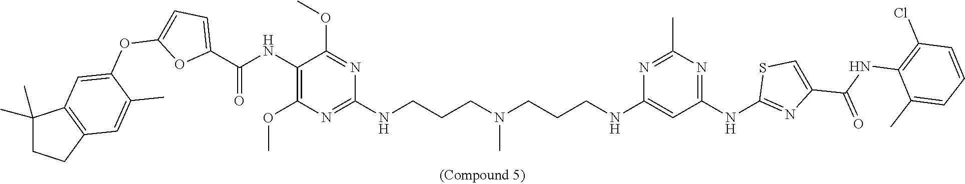 | (M + H)⁺ = 961.2 |
| 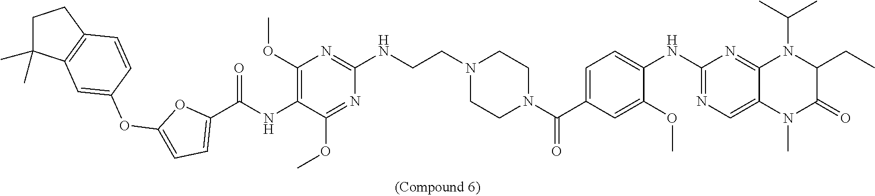 | (M + H)⁺ = 932.3 |
| 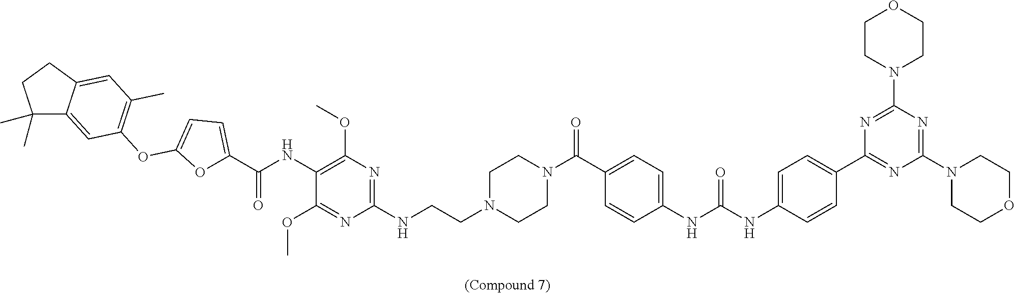 [SMILES: COc1nc(NCCN2CCN(C(=O)c3ccc(NC(=O)Nc4ccc(-c5nc(N6CCOCC6)nc(N6CCOCC6)n5)cc4)cc3)CC2)nc(OC)c1NC(=O)c1ccc(Oc2cc3c(cc2C)CCC3(C)C)o1] | (M + H)⁺ = 1038.5 |
| 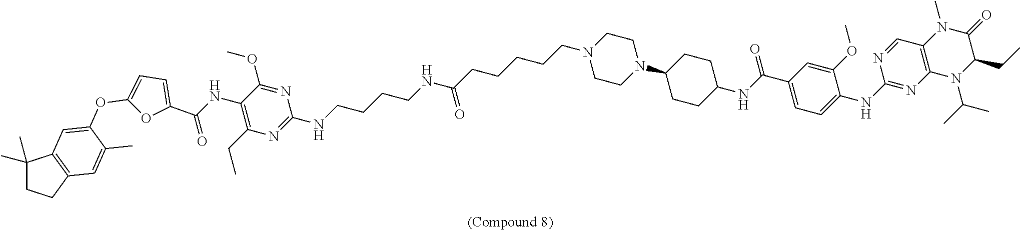 | (M + H)⁺ = 1170.6 |

### Example 2. N-[2-({2-[4-(2-{[(E)-{[(19S)-19-ethyl-19-hydroxy-14,18-dioxo-17-oxa-3,13-diazapentacyclo[11.8.0.0²,¹¹0.0⁴,⁹.0¹⁵,²⁰]henicosa-1(21),2(11),3,5,7,9,15(20)-heptaen-10-yl]methylidene}amino]oxy]acetyl)piperazin-1-yl]ethyl}amino)-4,6-dimethoxypyrimidin-5-yl]-5-[(3,3,6-trimethyl-2,3-dihydro-1H-inden-5-yl)oxy]furan-2-carboxamide (Compound 9)

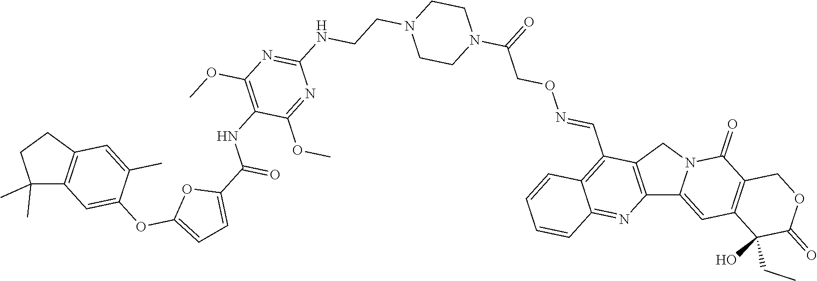 [SMILES: CC[C@@]1(O)C(=O)OCc2c1cc1n(c2=O)Cc2c-1nc1ccccc1c2/C=N/OCC(=O)N1CCN(CCNc2nc(OC)c(NC(=O)c3ccc(Oc4cc5c(cc4C)CCC5(C)C)o3)c(OC)n2)CC1]

Step 2-1, preparation of tert-butyl 4-{2-[(4,6-dimethoxy-5-{5-[(3,3,6-trimethyl-2,3-dihydro-1H-inden-5-yl)oxy]furan-2-amido}pyrimidin-2-yl)amino]ethyl}piperazine-1-carboxylate: to a DMSO (2.0 mL) solution of N-(2-chloro-4,6-dimethoxypyrimidin-5-yl)-5-[(3,3,6-trimethyl-2,3-dihydro-1H-inden-5-yl)oxy]furan-2-carboxamide (457 mg, 1.0 Eq, 1.0 mml) was added tert-butyl 4-(2-aminoethyl)piperazine-1-carboxylate (230 mg, 1.0 Eq, 1.0 mmol) and N,N-diisopropylethylamine (DIPEA) (1.74 mL, 10 Eq, 10 mmol). The resulting mixture was stirred at 120° C. for 8 hours. The reaction crude was diluted with water, extracted with ethyl acetate, washed with brine, dried, and concentrated. The remaining residue was purified by silica gel chromatography eluting with MeOH/DCM (0-5%) give tert-butyl 4-{2-[(4,6-dimethoxy-5-{5-[(3,3,6-trimethyl-2,3-dihydro-1H-inden-5-yl)oxy]furan-2-amido}pyrimidin-2-yl)amino]ethyl}piperazine-1-carboxylate (560 mg, 86%).

Step 2-2, preparation of N-(4,6-dimethoxy-2-{[2-(piperazin-1-yl)ethyl]amino}pyrimidin-5-yl)-5-[(3,3,6-trimethyl-2,3-dihydro-1H-inden-5-yl)oxy]furan-2-carboxamide: to a DCM (3.0 mL) solution of tert-butyl 4-{2-[(4,6-dimethoxy-5-{5-[(3,3,6-trimethyl-2,3-dihydro-1H-inden-5-yl)oxy]furan-2-amido}pyrimidin-2-yl)amino]ethyl}piperazine-1-carboxylate (560 mg, 1.0 Eq, 0.86 mmol) was added TFA (1.5 mL, 23.0 Eq, 19.7 mmol). The resulting mixture was stirred at ambient temperature for 24 hours. The reaction crude was concentrated, diluted with ethyl acetate, washed with saturated K₂CO₃, dried, and concentrated to yield crude N-(4,6-dimethoxy-2-{[2-(piperazin-1-yl)ethyl]amino}pyrimidin-5-yl)-5-[(3,3,6-trimethyl-2,3-dihydro-1H-inden-5-yl)oxy]furan-2-carboxamide (300 mg, 63%). This material was used for next step without further purification.

Step 2-3, preparation of 2-{[(E)-{[(19S)-19-ethyl-19-hydroxy-14,18-dioxo-17-oxa-3,13-diazapentacyclo[11.8.0.0²,¹¹0.0⁴,⁹0.0¹⁵,²⁰]henicosa-1(21),2(11),3,5,7,9,15(20)-heptaen-10-yl]methylidene}amino]oxy}acetic acid: to a MeOH (15 mL) solution of (19S)-19-ethyl-19-hydroxy-14,18-dioxo-17-oxa-3,13-diazapentacyclo[11.8.0.0²,¹¹0.0⁴,⁹0¹⁵,²⁰]henicosa-1(21),2(11),3,5,7,9,15(20)-heptaene-10-carbaldehyde (188 mg, 1.0 Eq, 0.50 mmol) was added the HCl salt of 2-(aminooxy)acetic acid (55 mg, 1.0 Eq, 0.50 mmol) and acetic acid (1.0 mL). The resulting mixture was stirred at ambient temperature for 1 hour. The resulting suspension was filtered, and the filtrate was washed with ethyl acetate/hexane (1:9) several times. The remaining solid was dried under vacuum to give crude 2-{[(E)-{[(19S)-19-ethyl-19-hydroxy-14,18-dioxo-17-oxa-3,13-diazapentacyclo[11.8.0.0²,¹¹0.0⁴,⁹0.0¹⁵,²⁰]henicosa-1(21),2(11),3,5,7,9,15(20)-heptaen-10-yl]methylidene}amino]oxy}acetic acid (43 mg, 20%). This material was used for next step without further purification.

Step 2-4, preparation of N-[2-({2-[4-(2-{1(E)-{[(19S)-19-ethyl-19-hydroxy-14,18-dioxo-17-oxa-3,13-diazapentacyclo[11.8.0.0²,¹¹0.0⁴,⁹0.0¹⁵, ²⁰]henicosa-1(21),2(11),3,5,7,9,15(20)-heptaen-10-yl]methylidene}amino]oxy}acetyl)piperazin-1-yl]ethyl}amino)-4,6-dimethoxypyrimidin-5-yl]-5-[(3,3,6-trimethyl-2,3-dihydro-1H-inden-5-yl)oxy]furan-2-carboxamide: to a DMF (1.0 mL) solution of crude 2-{[(E)-{[(19S)-19-ethyl-19-hydroxy-14,18-dioxo-17-oxa-3,13-diazapentacyclo[11.8.0.0²,¹¹0.0⁴,⁹0.0¹⁵,²⁰]henicosa-1(21),2(11),3,5,7,9,15(20)-heptaen-10-yl]methylidene}amino]oxy}acetic acid (22 mg, 1.0 Eq, 0.050 mmol) was added N,N-diisopropylethylamine (DIPEA) (18 μL, 2.0 Eq, 0.10 mmol), N,N,N′,N′-tetramethyl-O-(1H-benzotriazol-1-yl)uronium hexafluorophosphate (HBTU) (38 mg, 2.0 Eq, 0.10 mmol) and crude N-(4,6-dimethoxy-2-{[2-(piperazin-1-yl)ethyl]amino}pyrimidin-5-yl)-5-[(3,3,6-trimethyl-2,3-dihydro-1H-inden-5-yl)oxy]furan-2-carboxamide (27 mg, 1.0 Eq, 0.050 mmol). The resulting mixture was stirred at ambient temperature for 2 hours. The reaction mixture was diluted with ethyl acetate, washed with water and bine, dried, and concentrated. The remaining residue was purified by prep-TLC eluting with DCM/MeOH (20/1) to give N-[2-({2-[4-(2-{[(E)-{[(19S)-19-ethyl-19-hydroxy-14,18-dioxo-17-oxa-3,13-diazapentacyclo[11.8.0.0²,¹¹ 0.0⁴,⁹0.0¹⁵,²⁰]henicosa-1(21),2(11),3,5,7,9,15(20)-heptaen-10-yl]methylidene}amino]oxy}acetyl)piperazin-1-yl]ethyl}amino)-4,6-dimethoxypyrimidin-5-yl]-5-[(3,3,6-trimethyl-2,3-dihydro-1H-inden-5-yl)oxy]furan-2-carboxamide (22 mg, 44%). MS (M+H)*=982.6.

The following compounds were prepared similarly to Example 2 with appropriate substituting reagents and substrates at different steps and they may require additional functional group modifications via well-known chemistry with appropriate reagents.

| | MS |
|---|---|
| 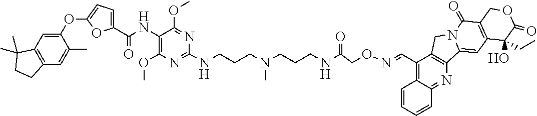 [SMILES: CC[C@@]1(O)C(=O)OCc2c1cc1n(c2=O)Cc2c-1nc1ccccc1c2/C=N/OCC(=O)NCCCN(C)CCCNc1nc(OC)c(NC(=O)c2ccc(Oc3cc4c(cc3C)CCC4(C)C)o2)c(OC)n1] | (M + H)⁺ = 999.5 |
| (Compound 10) | |

### Example 3.2-{[6-(16-{4-[4-(4-aminopiperidin-1-yl)-3-(5-chloro-1H-1,3-benzodiazol-2-yl)-5-(3-fluoro-5-methylphenyl)pyridin-2-ylpiperazin-1-yl}-16-oxo-4,7,10,13-tetraoxa-1-azahexadecan-1-yl)-2-methylpyrimidin-4-yl]amino}-N-(2-chloro-6-methylphenyl)-1,3-thiazole-5-carboxamide (Compound 11)

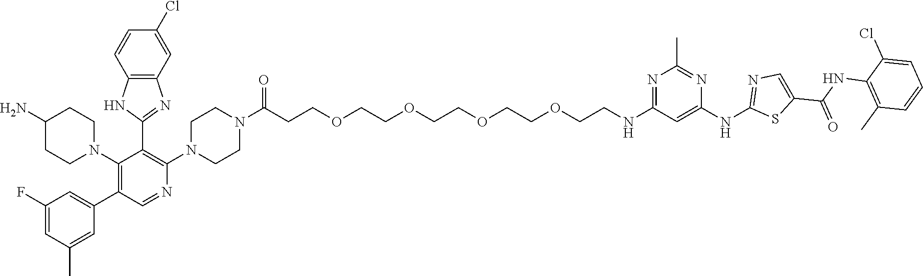 [SMILES: Cc1cc(F)cc(-c2cnc(N3CCN(C(=O)CCOCCOCCOCCOCCNc4cc(Nc5ncc(C(=O)Nc6c(C)cccc6Cl)s5)nc(C)n4)CC3)c(-c3nc4cc(Cl)ccc4[nH]3)c2N2CCC(N)CC2)c1]

Step 3-1, preparation of tert-butyl 1-{[6-({5-[(2-chloro-6-methylphenyl)carbamoyl]-1,3-thiazol-2-yl}amino)-2-methylpyrimidin-4-yl]amino}-3,6,9,12-tetraoxapentadecan-15-oate: to a DMSO (1.0 mL) solution of 2-[(6-chloro-2-methylpyrimidin-4-yl)amino]-N-(2-chloro-6-methylphenyl)-1,3-thiazole-5-carboxamide (78 mg, 1.0 Eq, 0.2 mmol) was added tert-butyl 1-amino-3,6,9,12-tetraoxapentadecan-15-oate (96 mg, 1.5 Eq, 0.30 mmol) and N,N-diisopropylethylamine (DIPEA) (0.30 mL, 8.6 Eq, 1.73 mmol). The resulting mixture was heated at 130° C. for 8 hours. The reaction crude was purified by C18 reversed phase eluting with MeCN (0.1% TFA)/water (0.1% TFA) (5-50%). Pure fractions were combined, neutralized with saturated NaHCO₃ (3 mL), added solid NaCl (5 g), and extracted with ethyl acetate (2×10 mL). Organic layers were combined, dried with MgSO₄, filtered, and concentrated to give tert-butyl 1-{[6-({5-[(2-chloro-6-methylphenyl)carbamoyl]-1,3-thiazol-2-yl}amino)-2-methylpyrimidin-4-yl]amino}-3,6,9,12-tetraoxapentadecan-15-oate (120 mg, 88%).

Step 3-2, preparation of 1-{[6-({5-[(2-chloro-6-methylphenyl)carbamoyl]-1,3-thiazol-2-yl}amino)-2-methylpyrimidin-4-yl]amino}-3,6,9,12-tetraoxapentadecan-15-oic acid: to a DCM (2.0 mL) solution of tert-butyl 1-{[6-({5-[(2-chloro-6-methylphenyl)carbamoyl]-1,3-thiazol-2-yl}amino)-2-methylpyrimidin-4-yl]amino}-3,6,9,12-tetraoxapentadecan-15-oate (120 mg, 1.0 Eq, 0.17 mmol) was added triethylsilane (0.25 mL, 10 Eq, 1.7 mmol) and TFA (0.48 mL, 40 Eq, 0.8 mL). The resulting mixture was stirred at ambient temperature for 1 hour. The reaction crude was concentrated, and MTBE was added. The top ether layer was decanted, and the remaining residue was dried under vacuum to afford the crude TFA salt of 1-{[6-({5-[(2-chloro-6-methylphenyl)carbamoyl]-1,3-thiazol-2-yl}amino)-2-methylpyrimidin-4-yl]amino}-3,6,9,12-tetraoxapentadecan-15-oic acid (90 mg, 72%). MS (M+H)⁺=623.4.

Step 3-3, preparation of benzyl N-{1-[3-(5-chloro-1H-1,3-benzodiazol-2-yl)-2-[4-(1-{[6-({5-[(2-chloro-6-methylphenyl)carbamoyl]-1,3-thiazol-2-yl}amino)-2-methylpyrimidin-4-yl]amino}-3,6,9,12-tetraoxapentadecanoyl)piperazin-1-yl]-5-(3-fluoro-5-methylphenyl)pyridin-4-yl]piperidin-4-yl}carbamate: to a DMF (1.0 mL) solution of 1-{[6-({5-[(2-chloro-6-methylphenyl)carbamoyl]-1,3-thiazol-2-yl}amino)-2-methylpyrimidin-4-yl]amino}-3,6,9,12-tetraoxapentadecan-15-oic acid (40 mg, 1.0 Eq, 0.064 mmol) was added N,N-diisopropylethylamine (DIPEA) (67 μL, 6.0 Eq, 0.38 mmol), 1-[bis(dimethylamino)methylene]-1H-1,2,3-triazolo[4,5-b]pyridinium 3-oxide hexafluoropbosphate (HATU) (49 mg, 2.0 Eq, 0.128 mmol) and (benzyl N-{1-[3-(5-chloro-1H-1,3-benzodiazol-2-yl)-5-(3-fluoro-5-methylphenyl)-2-(piperazin-1-yl)pyridin-4-yl]piperidin-4-yl}carbamate (50 mg, 1.1 Eq, 0.077 mmol). The resulting mixture was stirred at ambient temperature for 1 hour. The reaction crude was purified by C18 reversed phase eluting with MeCN (0.1% TFA)/water (0.1% TFA) (5-50%). Pure fractions were combined, neutralized with saturated NaHCO₃ (3 mL), added solid NaCl (5 g), and extracted with ethyl acetate (2×10 mL). Organic layers were combined, dried with MgSO₄, filtered, and concentrated to give benzyl N-{1-[3-(5-chloro-1H-1,3-benzodiazol-2-yl)-2-[4-(1-{[6-({5-[(2-chloro-6-methylphenyl)carbamoyl]-1,3-thiazol-2-yl}amino)-2-methylpyrimidin-4-yl]amino}-3,6,9,12-tetraoxapentadecanoyl)piperazin-1-yl]-5-(3-fluoro-5-methylphenyl)pyridin-4-yl]piperidin-4-yl}carbamate (45 mg, 56%). MS (½M+H)⁺=630.7.

Step 3-4, preparation of 2-{[6-(16-{4-[4-(4-aminopiperidin-1-yl)-3-(5-chloro-1H-1,3-benzodiazol-2-yl)-5-(3-fluoro-5-methylphenyl)pyridin-2-yl]piperazin-1-yl}-16-oxo-4,7,10,13-tetraoxa-1-azahexadecan-1-yl)-2-methylpyrimidin-4-yl]amino}-N-(2-chloro-6-methylphenyl)-1,3-thiazole-5-carboxamide: to a TFA (1.0 mL) solution of benzyl N-{1-[3-(5-chloro-1H-1,3-benzodiazol-2-yl)-2-[4-(1-{[6-({5-[(2-chloro-6-methylphenyl)carbamoyl]-1,3-thiazol-2-yl}amino)-2-methylpyrimidin-4-yl]amino}-3,6,9,12-tetraoxapentadecanoyl)piperazin-1-yl]-5-(3-fluoro-5-methylphenyl)pyridin-4-yl]piperidin-4-yl}carbamate (45 mg, 1.0 Eq, 0.036 mmol) was added thioanisole (60 μL). The resulting mixture was heated at 60° C. for 1 hour. The reaction crude was purified by C₁₈ reversed phase eluting with MeCN (0.1% TFA)/water (0.1% TFA) (5-50%). Pure fractions were combined, neutralized with saturated NaHCO₃ (3 mL), added solid NaCl (5 g), and extracted with ethyl acetate (2×10 mL). Organic layers were combined, dried with MgSO₄, filtered, and concentrated with HCl in ethyl ether (0.1 mL, 2.0 N) to give the HCl salt of 2-{[6-(16-{4-[4-(4-aminopiperidin-1-yl)-3-(5-chloro-1H-1,3-benzodiazol-2-yl)-5-(3-fluoro-5-methylphenyl)pyridin-2-yl]piperazin-1-yl}-16-oxo-4,7,10,13-tetraoxa-1-azahexadecan-1-yl)-2-methylpyrimidin-4-yl]amino}-N-(2-chloro-6-methylphenyl)-1,3-thiazole-5-carboxamide (15 mg, 28%). MS (½M+H)⁺=563.0.

The following compounds were prepared similarly to Example 3 with appropriate substituting reagents and substrates at different steps and they may require additional functional group modifications via well-known chemistry with appropriate reagents.

| Compound | MS |
|---|---|
| 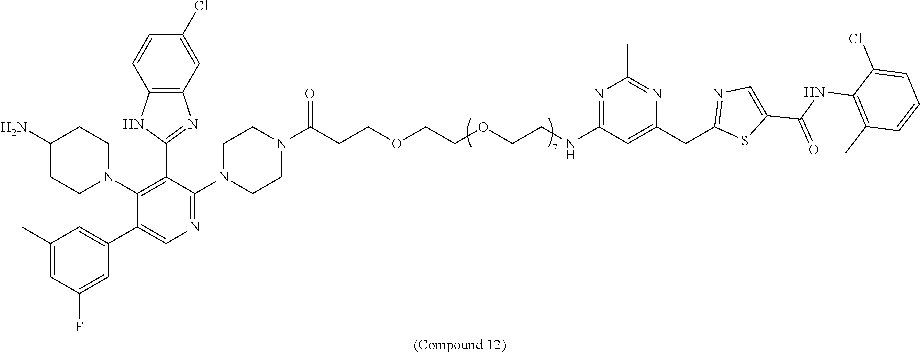 | (1/2M + H)⁺ = 651.1 |
| 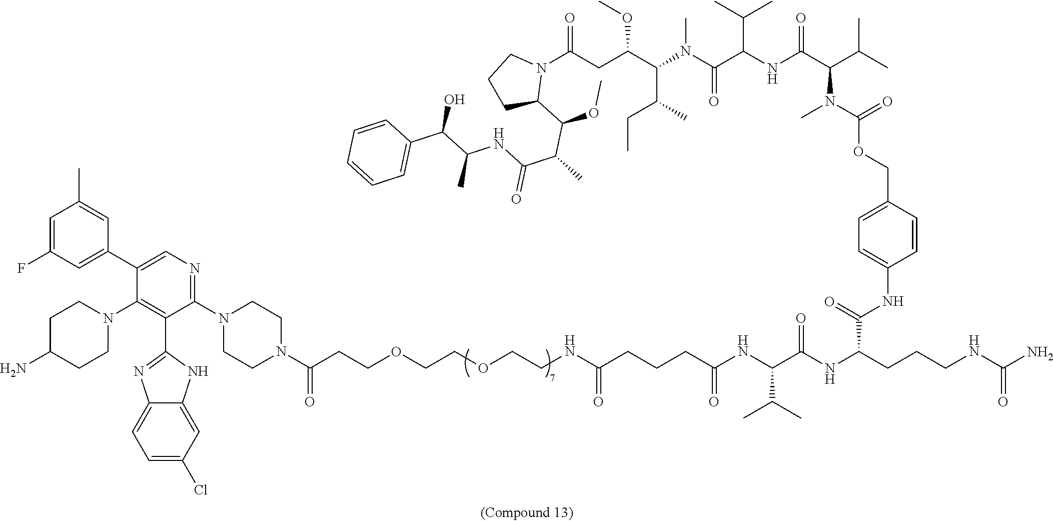 | (M + H)⁺ = 2164.0 |
| 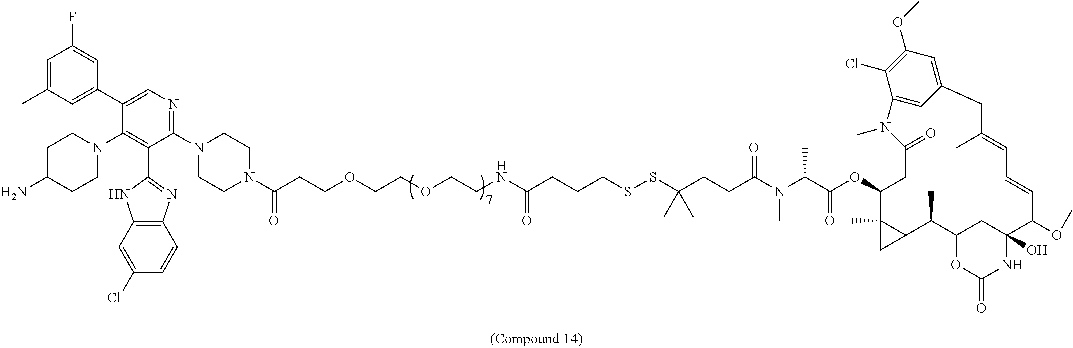 | (M + H)⁺ = 1823.2 |

### Example 4. (2S)-30-{4-[4-(4-aminopiperidin-1-yl)-3-(6-chloro-1H-1,3-benzodiazol-2-yl)-5-(3-fluoro-5-methylphenyl)pyridin-2-yl]piperazin-1-yl}-N-f(1S)-1-1{[3 r, 4S,5S)-3-methoxy-1-f(2S)-2-f(1R,2R)-1-methoxy-2-methyl-2-1f2-(pyridin-2-yl)ethyl]carbamoyl}ethyl]pyrrolidin-1-yl]-5-methyl-1-oxoheptan-4-yl](methyl)carbamoyl]-2-methylpropyl}-3-methyl-30-oxo-2-(propan-2-yl)-6,9,12,15,18,21,24,27-octaoxa-3-azatriacontanamide (Compound 17)

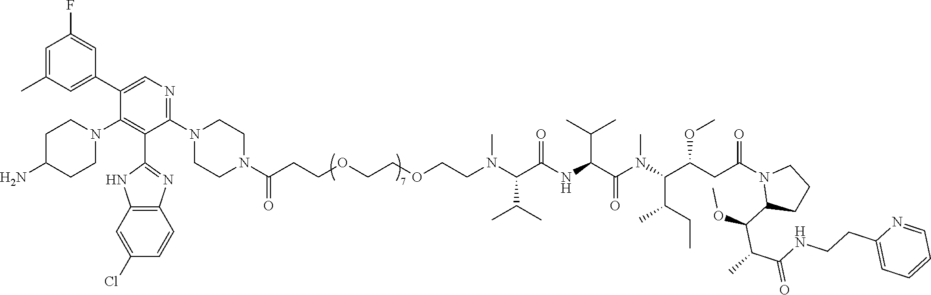

Step 4-1, preparation of benzyl N-{1-[3-(6-chloro-1H-1,3-benzodiazol-2-yl)-5-(3-fluoro-5-methylphenyl)-2-{4-[(1S)-1-{[(1S)-1-{[(3R,4S,5S)-3-methoxy-1-[(2S)-2-[(1R,2R)-1-methoxy-2-methyl-2-{[2-(pyridin-2-yl)ethyl]carbamoyl}ethyl]pyrrolidin-1-yl]-5-methyl-1-oxoheptan-4-yl](methyl)carbamoyl}-2-methylpropyl}carbamoyl}-2-methyl-1-(propan-2-yl)-5,8,11,14,17,20,23,26-octaoxa-2-azanonacosanoyl]piperazin-1-yl}pyridin-4-yl]piperidin-4-yl}carbamate: to a DMF (0.5 mL) solution of (29S)-29-{[(1S)-1-{[(3R,4S,5S)-3-methoxy-1-[(2S)-2-[(1R,2R)-1-methoxy-2-methyl-2-{[2-(pyridin-2-yl)ethyl]carbamoyl}ethyl]pyrrolidin-1-yl]-5-methyl-1-oxoheptan-4-yl](methyl)carbamoyl}-2-methylpropyl]carbamoyl}-28,30-dimethyl-4,7,10,13,16,19,22,25-octaoxa-28-azahentriacontanoic acid (12 mg, 1.0 Eq, 0.010 mmol) was added N,N-diisopropylethylamine (DIPEA) (10 μL, 5 Eq, 0.050 mmol), 1-[bis(dimethyl amino)methylene]-1H-1,2,3-triazolo[4,5-b]pyridinium 3-oxide hexafluorophosphate (HATU) (8.0 mg, 2.0 Eq, 0.020 mmol) and (benzyl N-{1-[3-(5-chloro-1H-1,3-benzodiazol-2-yl)-5-(3-fluoro-5-methylphenyl)-2-(piperazin-1-yl)pyridin-4-yl]piperidin-4-yl}carbamate (12 mg, 2.0 Eq, 0.020 mmol). The resulting mixture was stirred at ambient temperature for 1 hour. The reaction crude was purified by C₁₈ reversed phase eluting with MeCN (0.1% TFA)/water (0.1% TFA) (5-50%). Pure fractions were combined, neutralized with saturated NaHCO₃ (3 mL), added solid NaCl (5 g), and extracted with ethyl acetate (2×10 mL). Organic layers were combined, dried with MgSO₄, filtered, and concentrated to give benzyl N-{1-[3-(6-chloro-1H-1,3-benzodiazol-2-yl)-5-(3-fluoro-5-methylphenyl)-2-{4-[(1S)-1-{[(1S)-1-{[(3R,4S,5S)-3-methoxy-1-[(2S)-2-[(1R,2R)-1-methoxy-2-methyl-2-{[2-(pyridin-2-yl)ethyl]carbamoyl}ethyl]pyrrolidin-1-yl]-5-methyl-1-oxoheptan-4-yl](methyl)carbamoyl}-2-methylpropyl]carbamoyl}-2-methyl-1-(propan-2-yl)-5,8,11,14,17,20,23,26-octaoxa-2-azanonacosanoyl]piperazin-1-yl}pyridin-4-yl]piperidin-4-yl}carbamate (10 mg, 57%). MS (½M+H)⁺=876.1.

Step 4-2, preparation of (2S)-30-{4-[4-(4-aminopiperidin-1-yl)-3-(6-chloro-1H-1,3-benzodiazol-2-yl)-5-(3-fluoro-5-methylphenyl)pyridin-2-yl]piperazin-1-yl}-N-[(1S)-1-{[(3R,4S,5S)-3-methoxy-1-[(2S)-2-[(1R,2R)-1-methoxy-2-methyl-2-{[2-(pyridin-2-yl)ethyl]carbamoyl}ethyl]pyrrolidin-1-yl]-5-methyl-1-oxoheptan-4-yl](methyl)carbamoyl}-2-methylpropyl}-3-methyl-30-oxo-2-(propan-2-yl)-6,9,12,15,18,21,24,27-octaoxa-3-azatriacontanamide: to a TFA (0.50 mL) solution of benzyl N-{1-[3-(6-chloro-1H-1,3-benzodiazol-2-yl)-5-(3-fluoro-5-methylphenyl)-2-{4-[(1S)-1-{[(1S)-1-{[(3R,4S,5S)-3-methoxy-1-[(2S)-2-[(1R,2R)-1-methoxy-2-methyl-2-{[2-(pyridin-2-yl)ethyl]carbamoyl}ethyl]pyrrolidin-1-yl]-5-methyl-1-oxoheptan-4-yl](methyl)carbamoyl}-2-methylpropyl]carbamoyl}-2-methyl-1-(propan-2-yl)-5,8,11,14,17,20,23,26-octaoxa-2-azanonacosanoyl]piperazin-1-yl}pyridin-4-yl]piperidin-4-yl}carbamate (10 mg, 1.0 Eq, 0.0057 mmol) was added thioanisole (20 μL). The resulting mixture was heated at 60° C. for 1 hour. The reaction crude was purified by C₁₈ reversed phase eluting with MeCN (0.1% TFA)/water (0.1% TFA) (5-50%). Pure fractions were combined and freeze-dried to give the TFA salt of (2S)-30-{4-[4-(4-aminopiperidin-1-yl)-3-(6-chloro-1H-1,3-benzodiazol-2-yl)-5-(3-fluoro-5-methylphenyl)pyridin-2-yl]piperazin-1-yl}-N-[(1S)-1-{[(3R,4S,5S)-3-methoxy-1-[(2S)-2-[(1R,2R)-1-methoxy-2-methyl-2-{[2-(pyridin-2-yl)ethyl]carbamoyl}ethyl]pyrrolidin-1-yl]-5-methyl-1-oxoheptan-4-yl](methyl)carbamoyl}-2-methylpropyl]-3-methyl-30-oxo-2-(propan-2-yl)-6,9,12,15,18,21,24,27-octaoxa-3-azatriacontanamide (6.6 mg, 72%). MS (½M+H)*=808.4.

The following compounds were prepared similarly to Example 4 with appropriate substituting reagents and substrates at different steps and additional functional group modifications via well-known chemistry with appropriate reagents as required.

| Compound | MS |
|---|---|
| 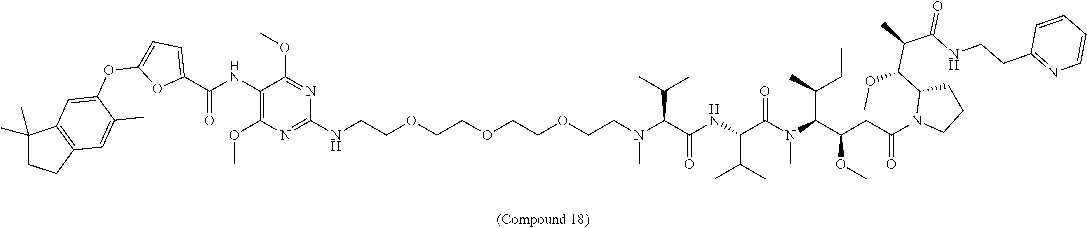 [SMILES: CC[C@H](C)[C@@H]([C@@H](CC(=O)N1CCC[C@H]1[C@H](OC)[C@@H](C)C(=O)NCCc1ccccn1)OC)N(C)C(=O)[C@@H](NC(=O)[C@H](C(C)C)N(C)CCOCCOCCOCCNc1nc(OC)c(NC(=O)c2ccc(Oc3cc4c(cc3C)CCC4(C)C)o2)c(OC)n1)C(C)C] | (M + H)⁺ = 1286.2 |
| 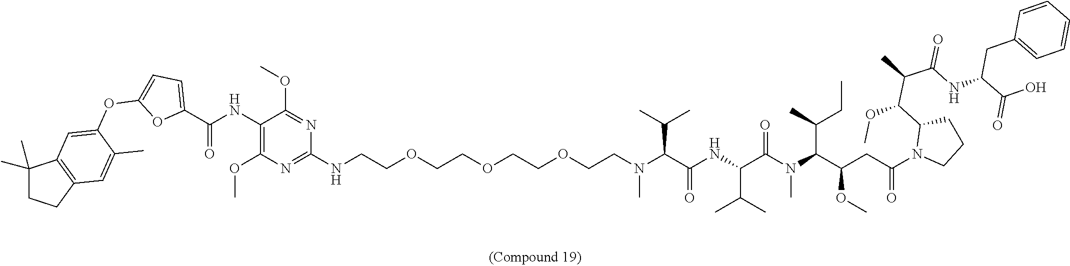 [SMILES: CC[C@H](C)[C@@H]([C@@H](CC(=O)N1CCC[C@H]1[C@H](OC)[C@@H](C)C(=O)N[C@H](Cc1ccccc1)C(=O)O)OC)N(C)C(=O)[C@@H](NC(=O)[C@H](C(C)C)N(C)CCOCCOCCOCCNc1nc(OC)c(NC(=O)c2ccc(Oc3cc4c(cc3C)CCC4(C)C)o2)c(OC)n1)C(C)C] | (M + H)⁺ = 1329.2 |

### Example A-1: Parenteral Pharmaceutical Composition

To prepare a parenteral pharmaceutical composition suitable for administration by injection (subcutaneous, intravenous), 0.001-500 mg of a compound Formula (I), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection

### Biology Examples

### Example B-1: SSTR Assays

### Functional Assay for SSTR2 Agonists

General overview: All five SSTR subtypes are Gi coupled G-protein coupled receptors (GPCRs) that lead to decreases in intracellular cyclic AMP (cAMP) when activated by an agonist. Therefore, measurement of intracellular cAMP levels can be used to assess whether compounds of the invention are agonists of SSTR subtypes (John Kelly, Troy Stevens, W. Joseph Thompson, and Roland Seifert, Current Protocols in Pharmacology, 2005, 2.2.1-2.2). One example of an intracellular cAMP assay is described below.

cAMP Assay Protocol

Four days prior to the assay, 5,000 Chinese hamster ovary cells (CHO-K1, ATCC #CCL-61) stably expressing the human somatostatin receptor subtype 2 are plated in each well of a 96-well tissue culture-treated plate in Ham's F12 growth media (ThermoFisher #10-080-CM) supplemented with 10% donor bovine serum (Gemini Bio-Products #100-506), 100 U/mL penicillin; 100 ug/mL streptomycin; 2 mM L-glutamine (Gemini Bio-Products #400-110) and 0.2 mg/mL hygromycin B (GoldBio #31282-04-9). The cells are cultured at 37° C., 5% CO₂ and 95% humidity. On the day of the assay, the media is aspirated and the cells are treated with 50 μL of 1.6 μM NKH477 (Sigma #N3290) plus various dilutions of compounds of the invention in assay buffer [1× Hank's Balanced Salt Solution (ThermoFisher #SH3058802), 0.5 mM HEPES pH 7.4, 0.1% bovine serum albumin, 0.2 mM 3-Isobutyl-1-methylxanthine (IBMX, VWR #200002-790)]. The cells are incubated for 20 minutes at 37° C. (the final concentration of the compounds of the invention are typically 0-10,000 nM). The cells are treated with 50 μL of lysis buffer (HRTF cAMP kit, Cisbio). The lysate is transferred to 384-well plates and cAMP detection and visualization antibodies are added and incubated for 1-24 hours at room temperature. The time-resolved fluorescent signal is read with a Tecan M1000Pro multiplate reader. The intracellular cAMP concentrations are calculated by regression to a standard curve and are plotted vs. the concentration of the compounds of the invention and the EC₅₀ of the compounds are calculated using standard methods. All data manipluations are in GraphPad Prism v8 (GraphPad, San Diego, CA).

### Example B-2: GnRHR Assays

### Functional Assay for GnRHR

General overview: GnRHR is a G_{q/11}-coupled receptor that mediates the action of the GnRH hoprmone by activating the phosphatidylinositol-calcium second messanger system. Activation of the GnRHR induces the accumulation of inositol monophosphate, a stable metabolite of IP-3, that can be characterized as a measure of agonistic activity (increase in IP-One) or antagonistic activity (blockade of IP-One accumulation) by compounds of the invention. One example of an intracellular IP-One assay used to characterize GnRHR antagonists is described below.

### IP-One Assay Protocol

24 hours prior to the assay, 30,000 FlpIn T-Rex 293 Cells (ThermoFisher #R78007) stably expressing the functional human GnRH receptor upon induction with tetracycline were plated in a 96-well tissue culture-treated plate in FlpIn T-Rex 293 Growth Medium [DMEM (Corning #10-013-CM) supplemented with 10% fetal bovine serum (Gemini Bio-Products #900-208), 100 U/mL penicillin; 100 μg/mL streptomycin; 2 mM L-glutamine (Gemini Bio-Products #400-110)]and 50 ng/mL tetracycline hydrocholoride (Sigma, T7660). Cells were cultured at 37° C., 5% CO₂ and 95% humidity. On the day of the assay, the growth media was discarded and the cells were treated with 50 μL of a dose response curve of GnRH (Bachem #4033013) in the presence of various concentrations of fixed compound in assay buffer [10 mM HEPES (Biopioneer Cat# C0113) pH 7.4, 1 mM CaCl₂ (Fisher Scientific BP510-100), 0.5 mM MgCl₂ (Sigma M8266-100G), 4.2 mM KCl (Fisher Scientific P330-500), 146 mM NaCl (Spectrum Chemical #SO155), 5.5 mM Glucose (Sigma G7528), 50 mM LiCl (Fisher Scientific L121-100), 0.1% bovine serum albumin (Fisher Scientific Cat #BP1600)] and incubated for 1 hour at 37° C. (the final concentrations of GnRH ranged from 0-250 nM and the final concentrations of the compound ranged from 0-10,000 nM). 50 μL of lysis buffer (HRTF IP-one kit, Cisbio) was added on top of the treatment to lyse the cells. The lysate was transferred to 384-well plates and IP-one detection and visualization antibodies were added and incubated for 1-24 hours at room temperature. The time-resolved fluorescent signal was read with a Tecan M1000Pro (Tecan) multiplate reader. The intracellular IP-one concentrations were calculated by regression to a standard curve and were plotted vs. the concentration of the GnRH agonists in the presence of various concentrations of antagonist and the K_{B} of the compounds were calculated using standard curve-fitting methods. All data manipulations were performed using GraphPad Prism v8 (GraphPad, San Diego, CA).

Illustrative biological activity of compounds is demonstrated in the following Tables.

TABLE B
Representative Activity

| | | | NP Ligand | Conjugate |
|---|---|---|---|---|
| | Peptide | | activity | Activity |
| Compound | GPCR | payload | EC₅₀ | (EC₅₀) |
| 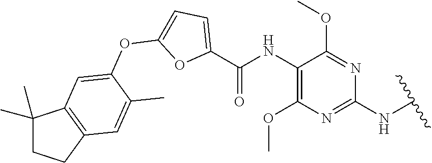 [SMILES: *Nc1nc(OC)c(NC(=O)c2ccc(Oc3cc4c(cc3C)CCC4(C)C)o2)c(OC)n1] 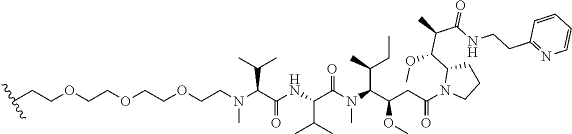 [SMILES: *CCOCCOCCOCCN(C)[C@H](C(=O)N[C@H](C(=O)N(C)[C@@H]([C@@H](C)CC)[C@@H](CC(=O)N1CCC[C@H]1[C@H](OC)[C@@H](C)C(=O)NCCc1ccccn1)OC)C(C)C)C(C)C] | GnRH | Auristatin | | 4.1 nM |
| 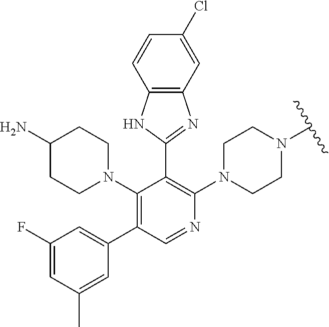 [SMILES: *N1CCN(c2ncc(-c3cc(C)cc(F)c3)c(N3CCC(N)CC3)c2-c2nc3cc(Cl)ccc3[nH]2)CC1] 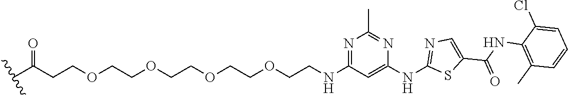 [SMILES: *C(=O)CCOCCOCCOCCOCCNc1cc(Nc2ncc(C(=O)Nc3c(C)cccc3Cl)s2)nc(C)n1] | SST2 | dasatinib | 8.7 nM | 0.091 nM |
| 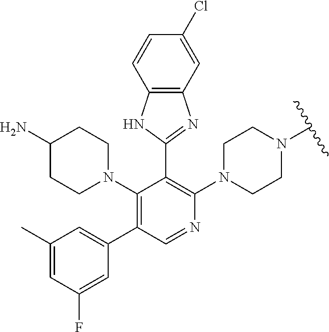 [SMILES: *N1CCN(c2ncc(-c3cc(C)cc(F)c3)c(N3CCC(N)CC3)c2-c2nc3cc(Cl)ccc3[nH]2)CC1] 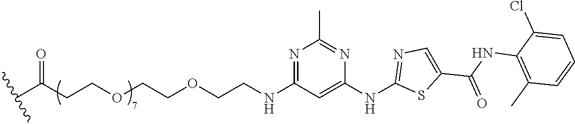 | SST2 | dasatinib | 8.7 nM | 0.048 nM |
| 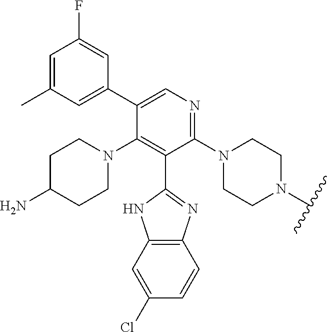 [SMILES: *N1CCN(c2ncc(-c3cc(C)cc(F)c3)c(N3CCC(N)CC3)c2-c2nc3ccc(Cl)cc3[nH]2)CC1] 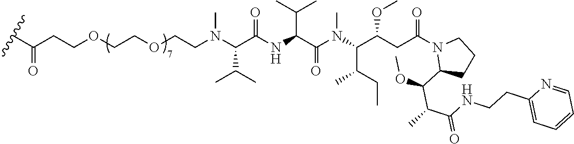 | SST2 | Auristatin | 8.7 nM | 1.6 nM |
| 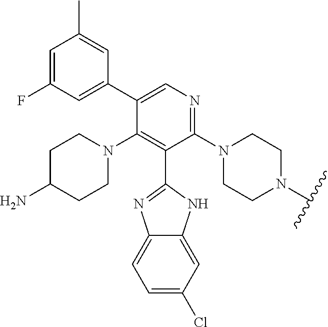 [SMILES: *N1CCN(c2ncc(-c3cc(C)cc(F)c3)c(N3CCC(N)CC3)c2-c2nc3ccc(Cl)cc3[nH]2)CC1] 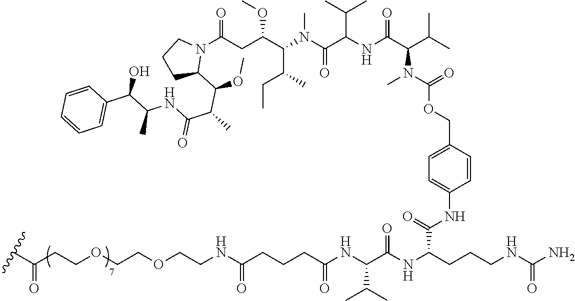 | SST2 | Auristatin | 8.7 nM | 1.5 nM |
| 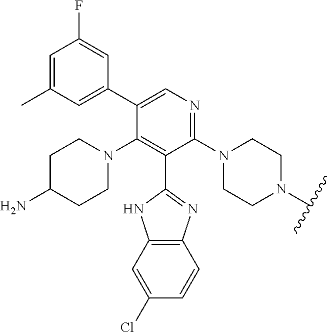 [SMILES: *N1CCN(c2ncc(-c3cc(C)cc(F)c3)c(N3CCC(N)CC3)c2-c2nc3ccc(Cl)cc3[nH]2)CC1] 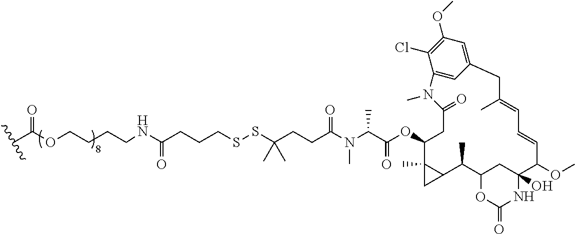 | SST2 | DM-4 | 8.7 nM | 0.74 nM |

The examples and embodiments described herein are for illustrative purposes only and various modifications or changes suggested to persons skilled in the art are to be included within the spirit and purview of this application and scope of the appended claims.

1. A compound of Formula (I), or a pharmaceutically acceptable salt thereof: 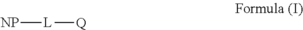
wherein:
(a) NP is a non-peptide ligand that binds to a gonadotropin-releasing hormone receptor (GnRHR) expressed in tumor cells;
(b) a payload moiety (Q) comprising a chemotherapeutic; and
(c) a linker (L) that covalently connects the non-peptide ligand NP and the payload moiety Q;
wherein NP has a structure of Formula (X), or a pharmaceutically acceptable salt thereof: 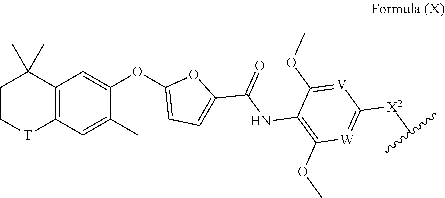
wherein:
V is CH or N; and W is CH or N;
T is absent, —CH₂—, —CH(CH₃)—, or —C(CH₃)₂—:
X² is absent, —O—, or —N(R⁷)—; and
R⁷ is hydrogen or substituted or unsubstituted C₁-C₆alkyl:
wherein upon administration to a mammal, the compound of Formula (I), or a pharmaceutically acceptable salt thereof, is targeted to tumor cells expressing the GnRHR.

2-13. (canceled)

14. The compound of claim 1, or a pharmaceutically acceptable salt thereof, wherein: NP has one of the following structures:

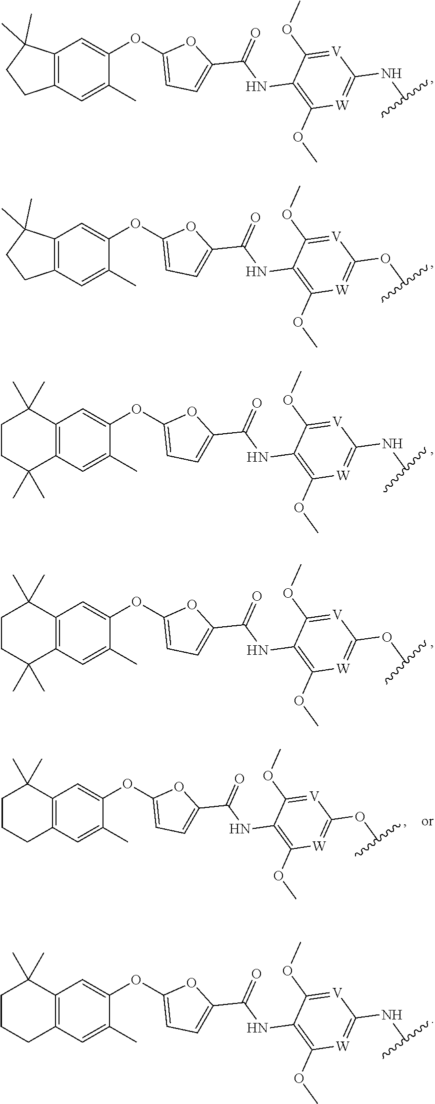

15. The compound of claim 1, wherein:
Q comprises a chemotherapeutic that is a cytotoxic drug, kinase inhibitor or both;
L is a non-cleavable linker or a cleavable linker.

16. The compound of claim 15, wherein:
the cleavable linker is an acid-sensitive linker, protease sensitivite linker, or glutathione-sensitive linker.

17. (canceled)

18. The compound of claim 15, wherein:
Q comprises a cytotoxic drug that is an antimitotic, DNA-damaging agent, transcriptional inhibitor or combination thereof.

19. The compound of claim 15, wherein:
Q comprises a cytotoxic drug that is an antimitotic, and wherein the antimitotic is a maytansinoid, taxane, auristatin, alkaloid, tubulysin, or epothilone.

20. The compound of claim 15, wherein:
Q comprises a kinase inhibitor that is an inhibitor of a cytoplasmic tyrosine kinase (CTK), a serine/threonine kinase (S/T Kinase), a lipid kinase (LK), or a receptor tyrosine kinase (RTK).

21. The compound of claim 15, or a pharmaceutically acceptable salt thereof, wherein, Q is:

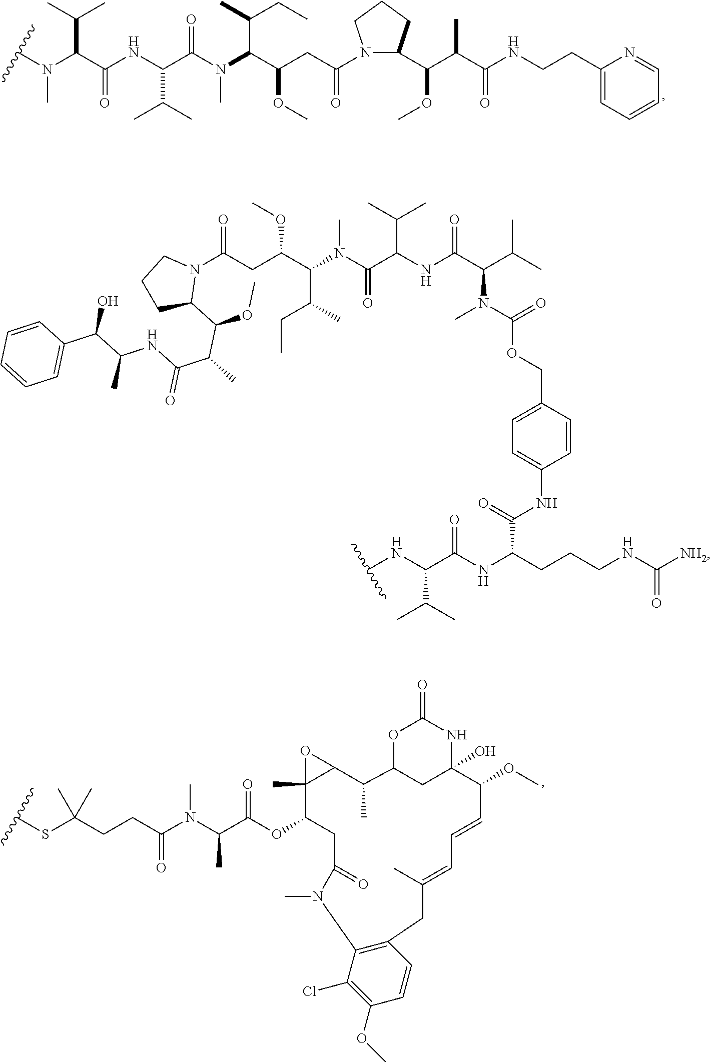 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)NCCc1ccccn1)OC)C(C)C)C(C)C ; *N[C@H](C(=O)N[C@@H](CCCNC(N)=O)C(=O)Nc1ccc(COC(=O)N(C)[C@@H](C(=O)NC(C(=O)N(C)[C@H]([C@H](C)CC)[C@H](CC(=O)N2CCC[C@@H]2[C@@H](OC)[C@H](C)C(=O)N[C@@H](C)[C@H](O)c2ccccc2)OC)C(C)C)C(C)C)cc1)C(C)C ; *SC(C)(C)CCC(=O)N(C)[C@H](C)C(=O)O[C@H]1CC(=O)N(C)c2c(ccc(OC)c2Cl)C/C(C)=C/C=C/[C@@H](OC)[C@@]2(O)CC(OC(=O)N2)[C@H](C)C2O[C@]21C]

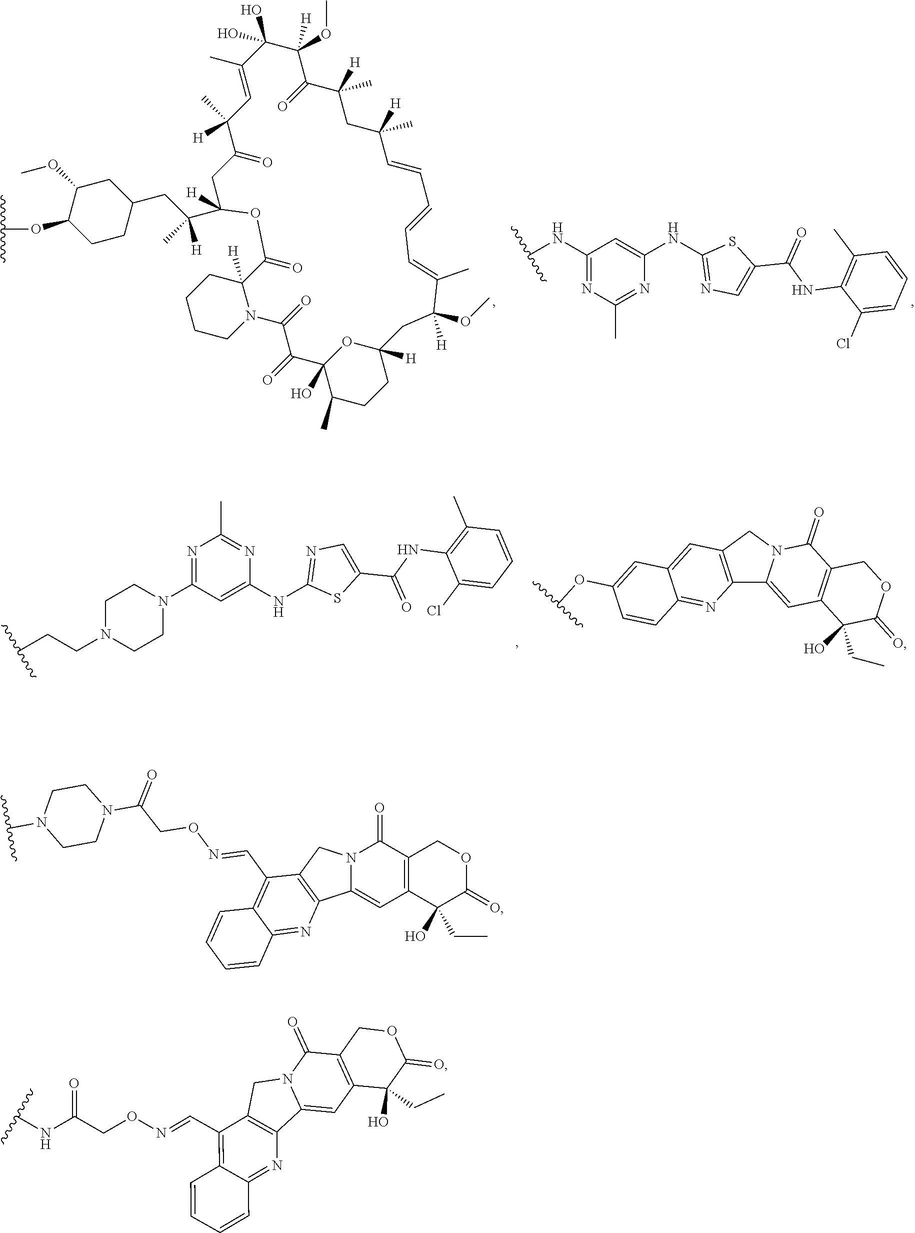

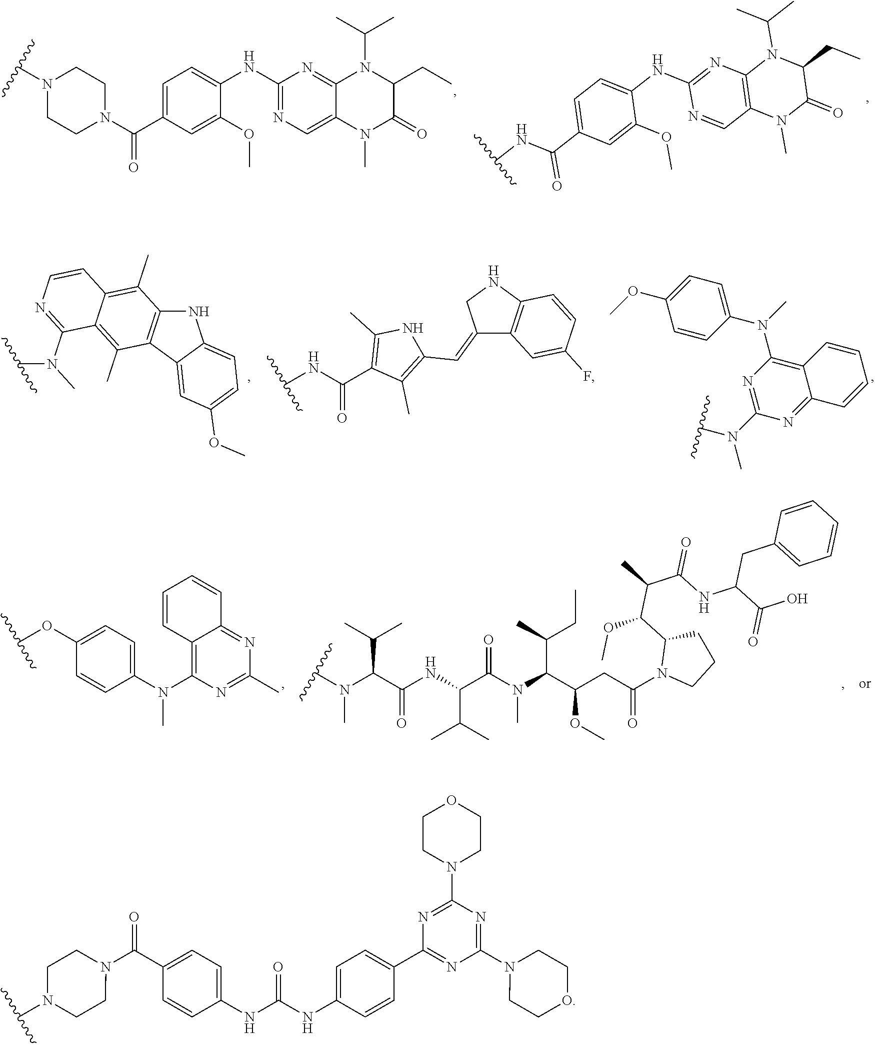

22. (canceled)

23. The compound of claim 15, wherein L is: 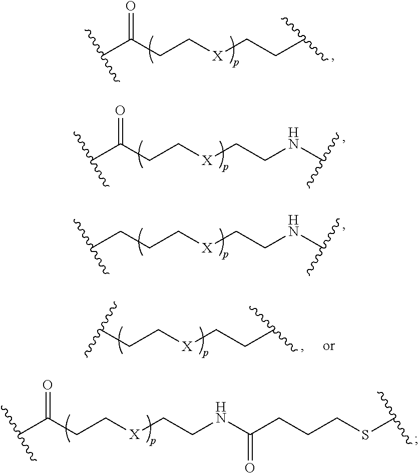
each p is independently 1, 2, 3, 4, 5, 6, 7, 8, 9, 10, 11, or 12;
each X is independently selected from O, and NR^{X}; and each R^{X} is independently selected from hydrogen, C₁-C₄alkyl and —CH₂CO₂H.

24. The compound of claim 15, wherein L is: 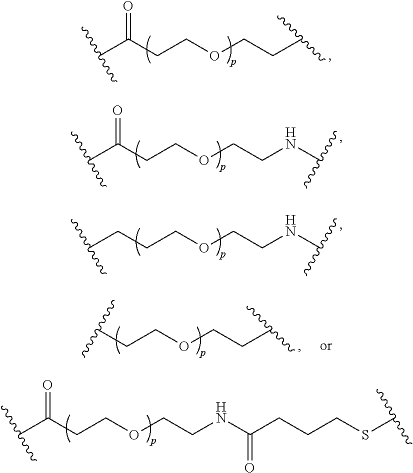
each p is independently 1, 2, 3, 4, 5, 6, 7, 8, 9, 10, 11, or 12.

25. The compound of claim 15, wherein L is: 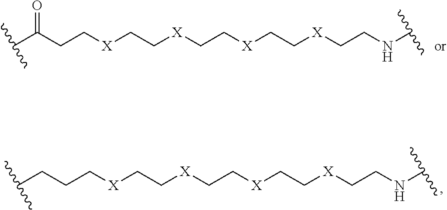
each X is independently selected from O, and NR^{X}; and each R^{X} is independently selected from hydrogen, C₁-C₄alkyl and —CH₂CO₂H.

26. The compound of claim 15, wherein L is:

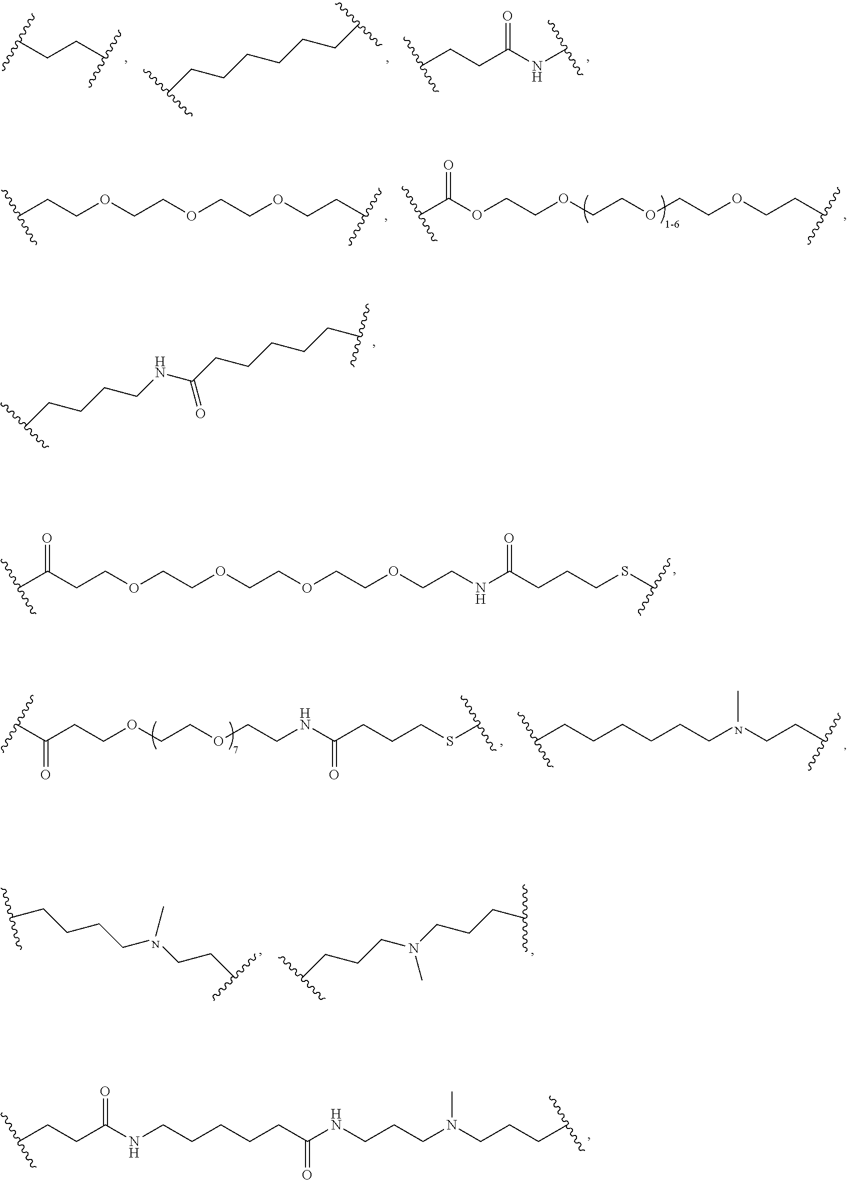

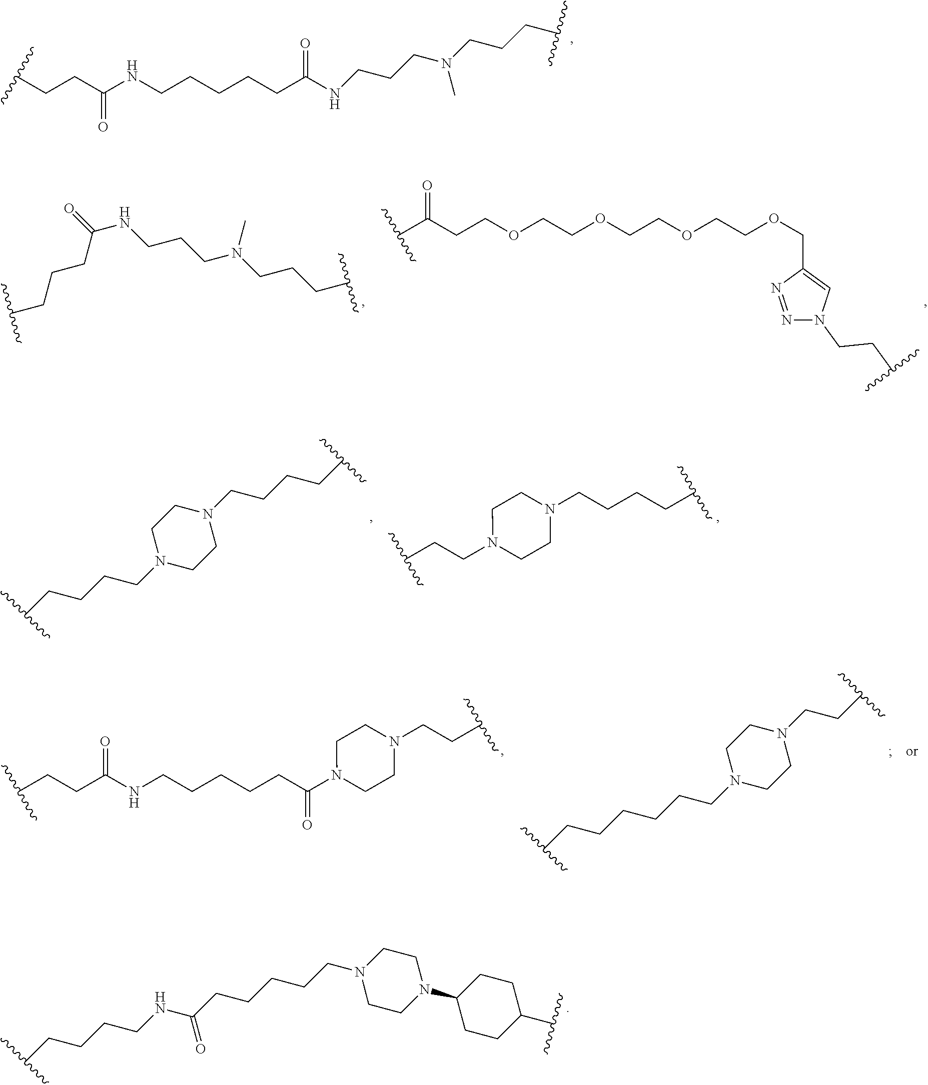

27. The compound of claim 1, or a pharmaceutically acceptable salt thereof, wherein, -L-Q is:

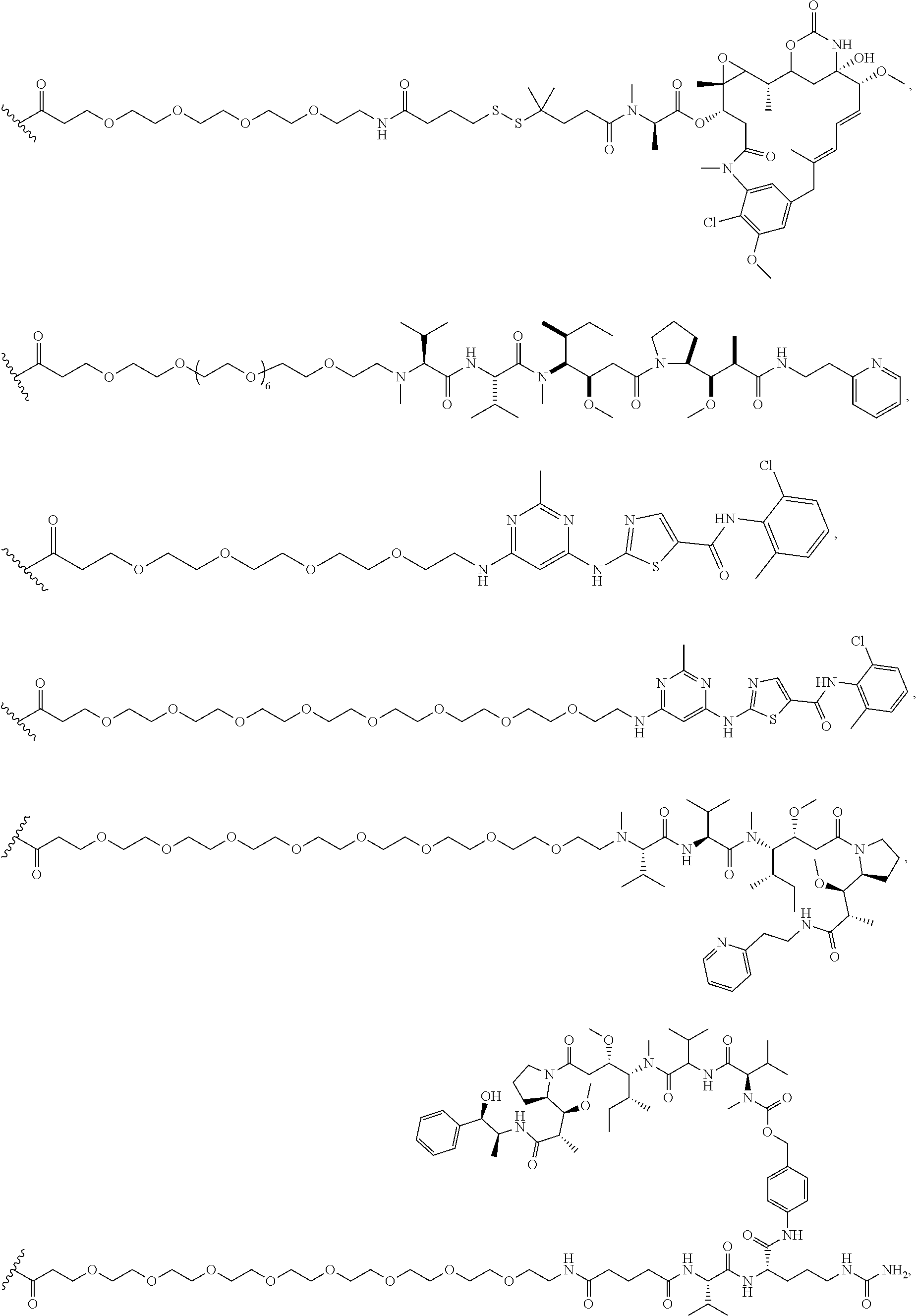

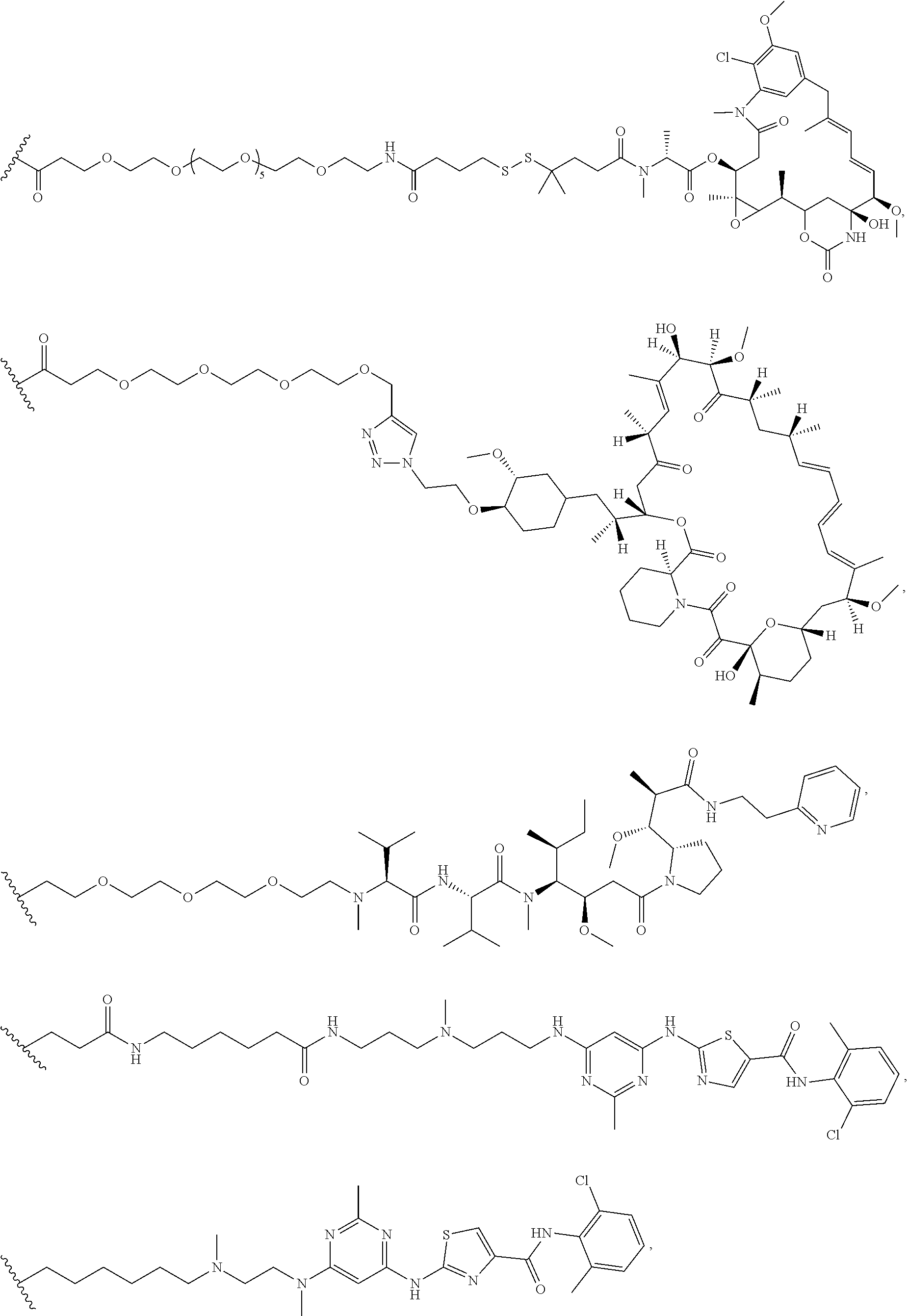

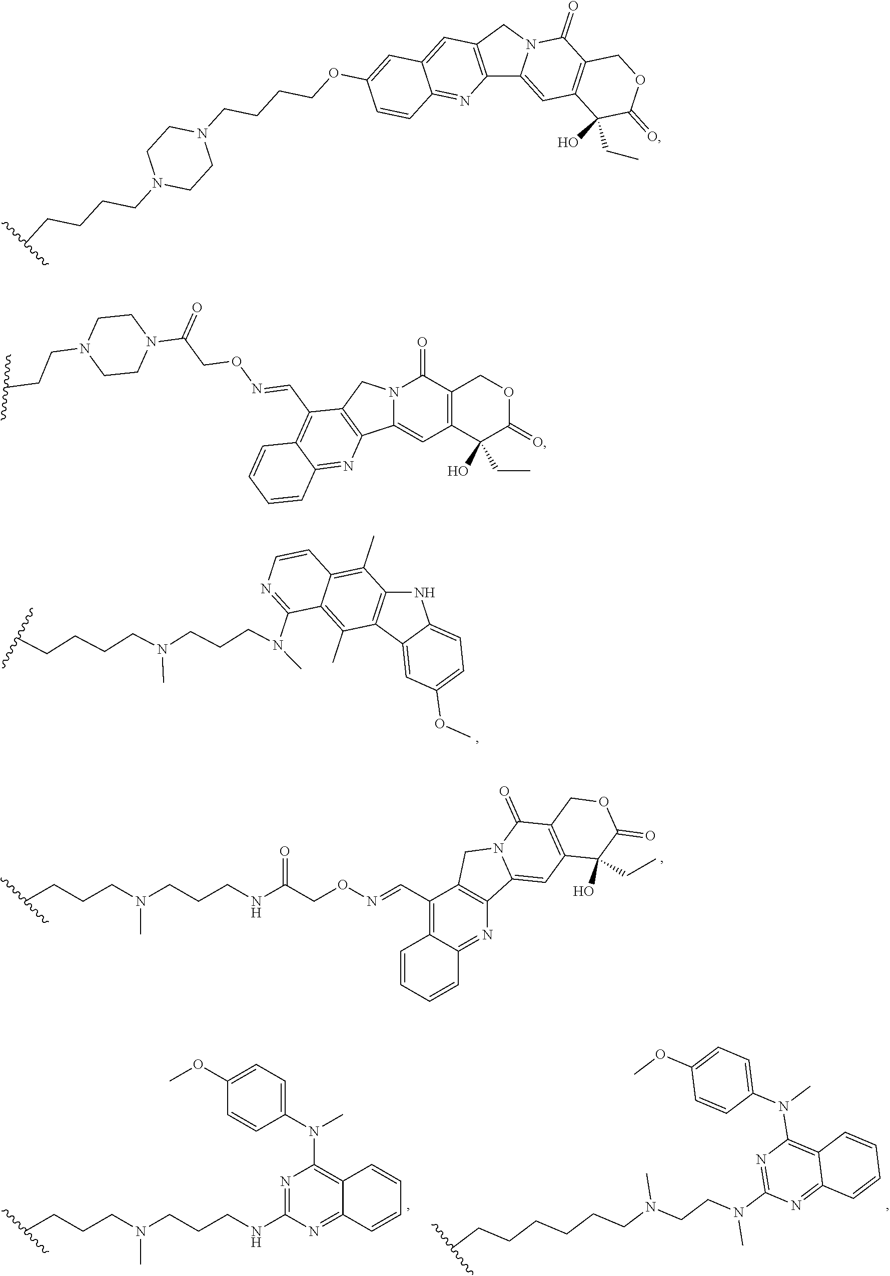 [SMILES: *CCCCN1CCN(CCCCOc2ccc3nc4c(cc3c2)Cn2c-4cc3c(c2=O)COC(=O)[C@]3(O)CC)CC1 ; *CCN1CCN(C(=O)CO/N=C/c2c3c(nc4ccccc24)-c2cc4c(c(=O)n2C3)COC(=O)[C@]4(O)CC)CC1 ; *CCCCN(C)CCCN(C)c1nccc2c(C)c3[nH]c4ccc(OC)cc4c3c(C)c12 ; *CCCN(C)CCCNC(=O)CO/N=C/c1c2c(nc3ccccc13)-c1cc3c(c(=O)n1C2)COC(=O)[C@]3(O)CC ; *CCCN(C)CCCNc1nc(N(C)c2ccc(OC)cc2)c2ccccc2n1 ; *CCCCCCN(C)CCN(C)c1nc(N(C)c2ccc(OC)cc2)c2ccccc2n1]

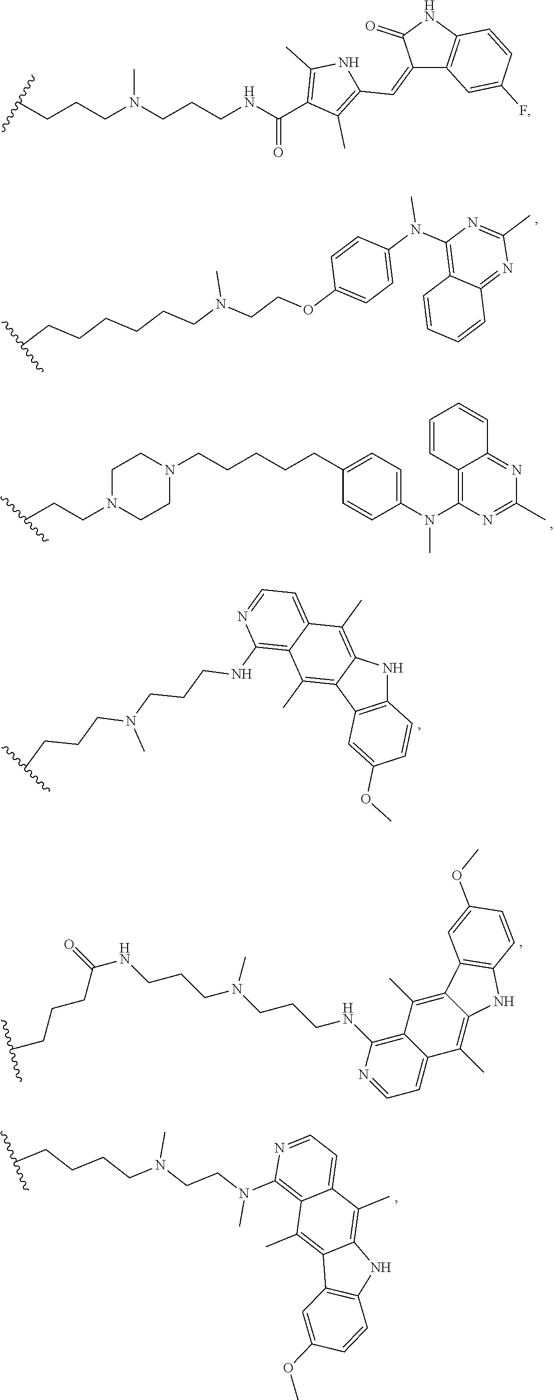 [SMILES: *CCCN(C)CCCNC(=O)c1c(C)[nH]c(/C=C2\C(=O)Nc3ccc(F)cc32)c1C ; *CCCCCCN(C)CCOc1ccc(N(C)c2nc(C)nc3ccccc23)cc1 ; *CCN1CCN(CCCCCc2ccc(N(C)c3nc(C)nc4ccccc34)cc2)CC1 ; *CCCN(C)CCCNc1nccc2c(C)c3[nH]c4ccc(OC)cc4c3c(C)c12 ; *CCCC(=O)NCCCN(C)CCCNc1nccc2c(C)c3[nH]c4ccc(OC)cc4c3c(C)c12 ; *CCCCN(C)CCN(C)c1nccc2c(C)c3[nH]c4ccc(OC)cc4c3c(C)c12]

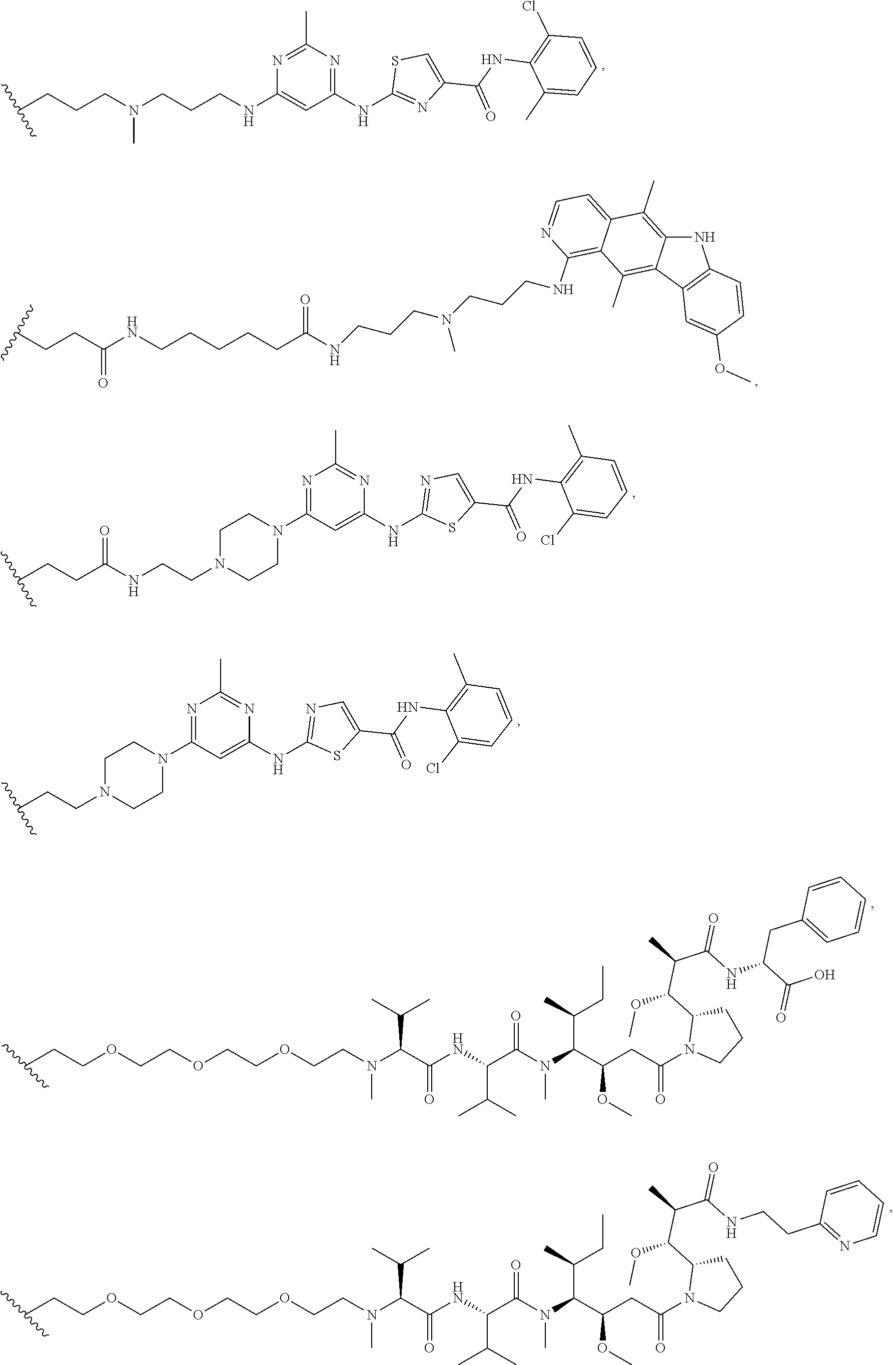

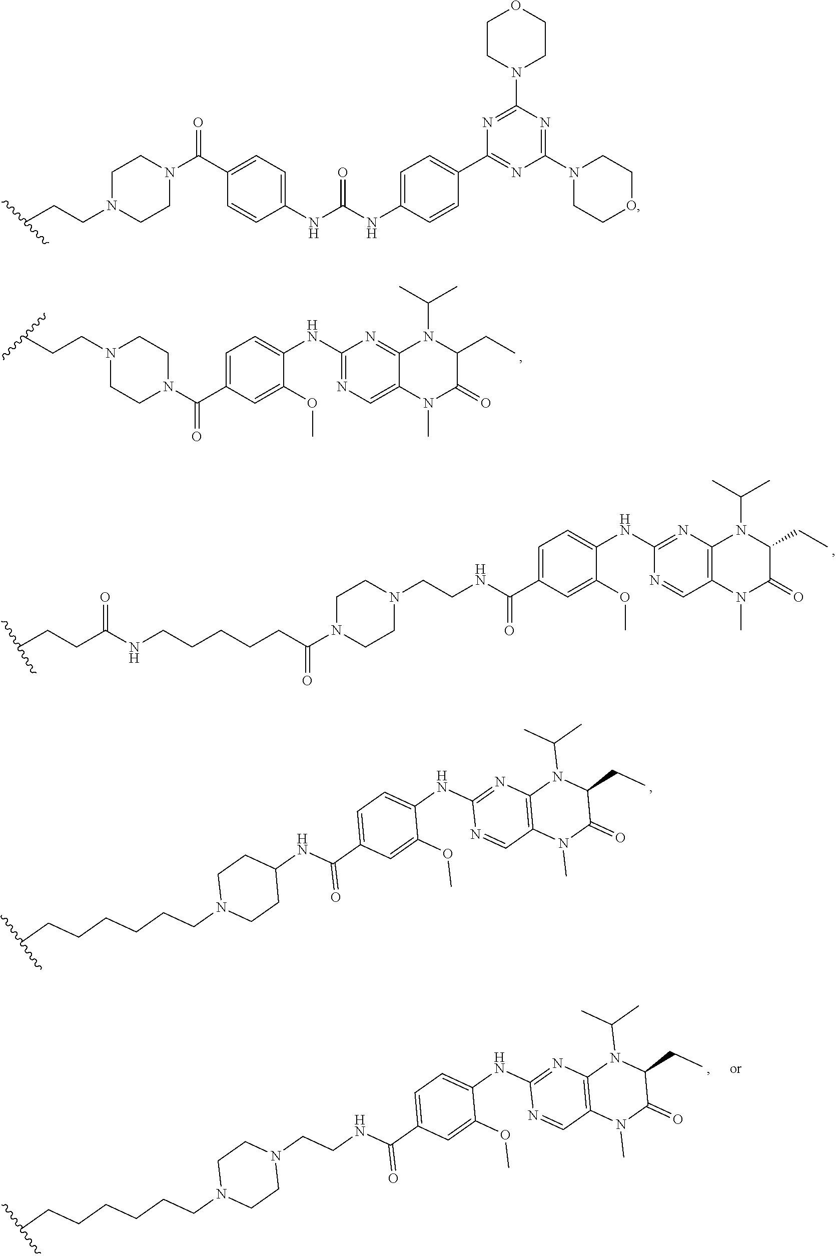 [SMILES: *CCN1CCN(C(=O)c2ccc(NC(=O)Nc3ccc(-c4nc(N5CCOCC5)nc(N5CCOCC5)n4)cc3)cc2)CC1 ; *CCN1CCN(C(=O)c2ccc(Nc3ncc4c(n3)N(C(C)C)C(CC)C(=O)N4C)c(OC)c2)CC1 ; *CCC(=O)NCCCCCC(=O)N1CCN(CCNC(=O)c2ccc(Nc3ncc4c(n3)N(C(C)C)[C@H](CC)C(=O)N4C)c(OC)c2)CC1 ; *CCCCCCN1CCC(NC(=O)c2ccc(Nc3ncc4c(n3)N(C(C)C)[C@@H](CC)C(=O)N4C)c(OC)c2)CC1 ; *CCCCCCN1CCN(CCNC(=O)c2ccc(Nc3ncc4c(n3)N(C(C)C)[C@@H](CC)C(=O)N4C)c(OC)c2)CC1]

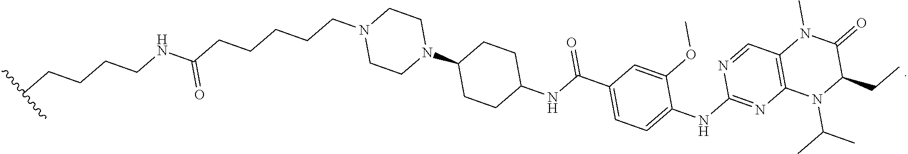

28. (canceled)

29. The compound of claim 15, or a pharmaceutically acceptable salt thereof, wherein the compound has one of the following structures: 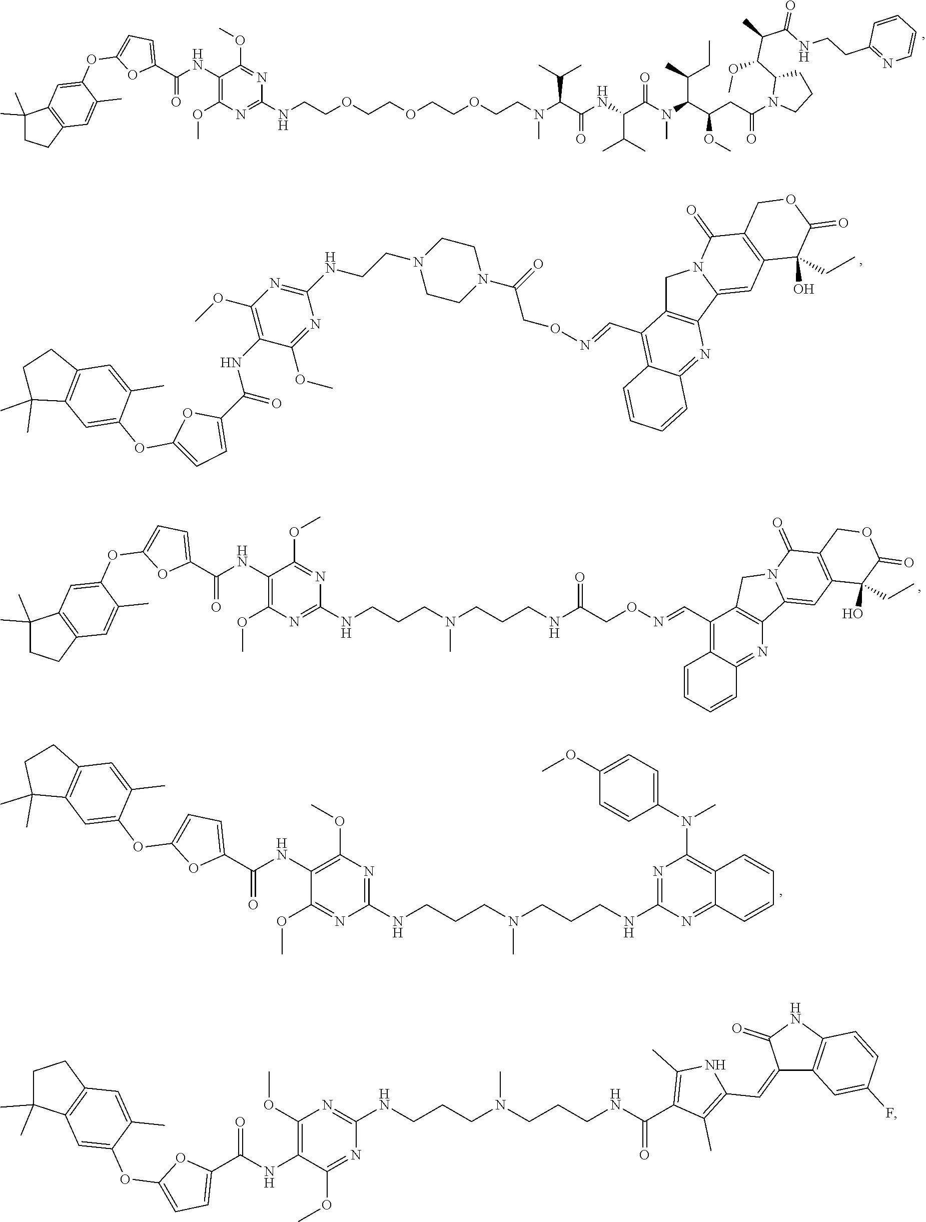 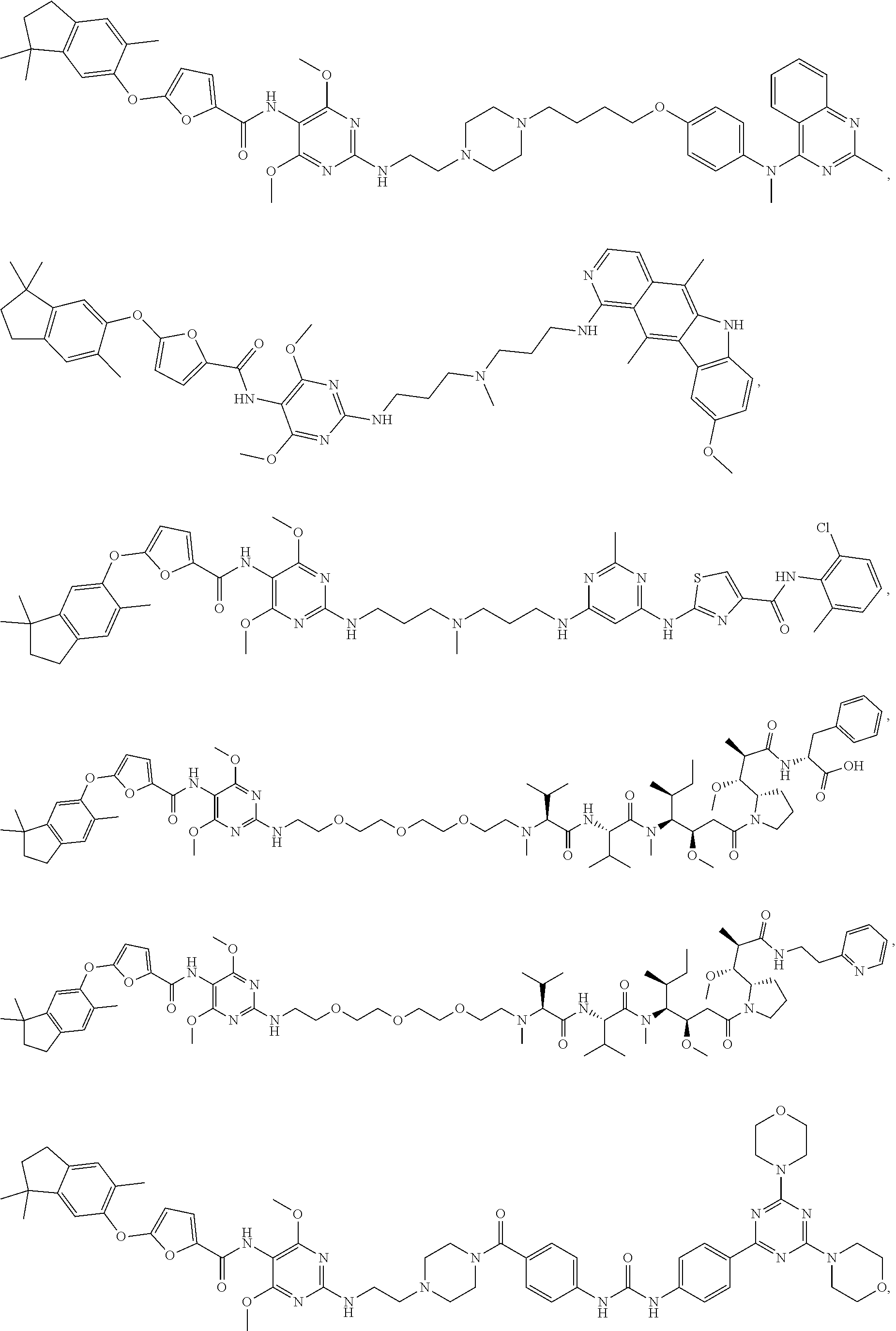 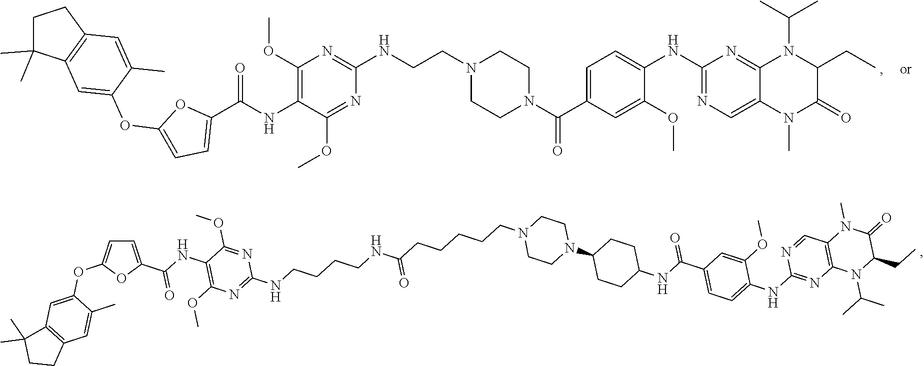
or a
pharmaceutically acceptable salt thereof.

30. A pharmaceutical composition comprising a compound of claim 1, or a pharmaceutically acceptable salt thereof, and at least one pharmaceutically acceptable excipient.

31. (canceled)

32. A method for the treatment of cancer comprising administering to a mammal with cancer an effective amount of a compound of claim 15, or a pharmaceutically acceptable salt thereof.

33-42. (canceled)
